SEQUENCE LISTING (13 sequences)

SEQ ID NO: 1 - DNA, 20 bp, Artificial Sequence
  Primer
ggagctgtcgtattccagtc

SEQ ID NO: 2 - DNA, 20 bp, Artificial Sequence
  Primer
aacccctcaagacccgttta

SEQ ID NO: 3 - protein, 294 aa, Mus musculus
MRLAVICFCLFGIASSLPVKVTDSGSSEEKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 4 - protein, 314 aa, Homo Sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 5 - protein, 19 aa, Artificial Sequence
  Synthetic Peptide based on human osteopontin
ELNGAYKAIPVAQDLNAPS

SEQ ID NO: 6 - protein, 5 aa, Artificial Sequence
  Synthetic Peptide based on human osteopontin
AIPVA

SEQ ID NO: 7 - protein, 20 aa, Artificial Sequence
  Synthetic Peptide based on human osteopontin
ELNGAYKAIPVAQDLNAPSD

SEQ ID NO: 8 - protein, 16 aa, Artificial Sequence
  Synthetic Peptide based on human osteopontin
PVAQDLNAPSDWDSRG

SEQ ID NO: 9 - protein, 34 aa, Artificial Sequence
  Synthetic Peptide based on human osteopontin
SHMESEELNGAYKAIPVAQDLNAPSDWDSRGKDS

SEQ ID NO: 10 - protein, 20 aa, Artificial Sequence
  Synthetic Peptide based on human osteopontin
KSHEEDKHLKFRISHELDGG

SEQ ID NO: 11 - protein, 20 aa, Artificial Sequence
  Synthetic Peptide based on human osteopontin
HLKFRISHELDSASSEVNGG

SEQ ID NO: 12 - protein, 20 aa, Artificial Sequence
  Synthetic Peptide based on human osteopontin
LDEHSSAISRSFEKVLNHGG

SEQ ID NO: 13 - protein, 20 aa, Artificial Sequence
  Synthetic Peptide based on human osteopontin
HLKFRISHELDSASSEVNGG

(19) United States
(12) Patent Application Publication
(10) Pub. No.: US 2010/0129376 A1
(43) Pub. Date: May 27, 2010

(54) Osteopontin Specific Antibodies and Methods of Use Thereof

(76) Inventors: David T. Denhardt, Bridgewater, NJ (US); Lawrence Steinman, Stanford, CA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/546,204

(22) Filed: Aug. 24, 2009

Related U.S. Application Data
(63) Continuation of application No. 12/187,373, filed on Aug. 6, 2008, now abandoned.
Application No. 12/187,373 is a continuation-in-part of application No. 10/188,884, filed on Jul. 2, 2002, now abandoned.
Application No. 10/188,884 is a division of application No. 09/340,484, filed on Jun. 30, 1999, now Pat. No. 6,414,219.
(60) Provisional application No. 60/091,200, filed on Jun. 30, 1998.
(60) Provisional application No. 60/963,642, filed on Aug. 6, 2007.

(51) Int. Cl. A61K 39/395; C07K 16/18; A61P 19/02; A61P 25/28; A61P 37/00; G01N 33/566; G01N 33/53

(52) U.S. Cl. 424/152.1; 530/388.2; 436/501; 435/7.1

(57) ABSTRACT

Monoclonal antibodies immunospecific for osteopontin are disclosed. Also provided are therapeutic methods of use thereof for modulating osteopontin levels for the treatment of autoimmune disorders.

This application is a continuation-in-part application of U.S. patent application Ser. No. 10/188,884, filed Jul. 2, 2002, which is a divisional application of Ser. No. 09/340,484 filed Jun. 30, 1999 now U.S. Pat. No. 6,414,219, which claims priority to U.S. Provisional Application 60/091,200 filed Jun. 30, 1998. This application also claims priority to US Provisional Application, 60/963,642 filed Aug. 4, 2007.

Pursuant to 35 U.S.C. §202(c) it is acknowledged that the U.S. Government has certain rights in the invention described herein, which was made in part with funds from the National Institutes of Health, Grant Number DC01295.

### FIELD OF THE INVENTION

This invention relates the fields of recombinant DNA technology, transgenic animals and production of clinicially valuable antibodies. More specifically, immunospecific antibodies which recognize different epitopes on osteopontin are provided as well as methods of use thereof in therapeutic applications.

### BACKGROUND OF THE INVENTION

Several publications and patent documents are cited throughout this application in order to more fully describe the state of the art to which this invention pertains. The disclosure of each of these publications is incorporated by reference herein.

Osteopontin (OPN) is a secreted phosphoprotein found in the collagenous extracellular matrix of mineralized tissues and in many body fluids, notably plasma, urine, bile and milk.⁽¹⁻³⁾ The protein has a GRGDS integrin-binding sequence that interacts with integrins of the αᵥ class, and it can facilitate attachment of cells to various surfaces, for example during the attachment of osteoclasts to bone.⁽⁴⁻⁵⁾ Sequence motifs in OPN that have been well conserved among avian and mammalian species include the RGD sequence just N-terminal to a thrombin cleavage site, an Asp-rich sequence with possible importance in binding to calcified tissues, a C-terminal heparin-binding domain, and multiple serine residues in contexts appropriate for phosphorylation by casein kinase II or mammary gland kinase.⁽⁶⁾ The synthesis of OPN is induced when T cells are activated,⁽⁷⁾ when JB6 epidermal cells are treated with 12-O-tetradecanoyl-phorbol-13-acetate⁽⁸⁾ and when Ras becomes activated and cells acquire a metastatic phenotype.⁽⁹⁾ Indeed, various experiments have shown that OPN is involved in the metastatic process.⁽¹⁰⁻¹²⁾

In addition to a cell attachment capability, OPN has properties of a cytokine.⁽⁷⁾ For example, it can activate c-src and stimulate phosphoinositide 3-kinase activity in target cells.^{(13,14)} OPN can inhibit the induction by lipopolysaccharide and γ-interferon of inducible nitric oxide synthase (iNOS, type II nitric oxide synthase).⁽¹⁵⁾ This inhibition of iNOS transcription correlates with the ability of OPN to protect tumor cells from being killed by activated macrophages,^{(16,17)} suggesting that perhaps this is how OPN contributes to the metastatic phenotype.⁽¹⁸⁾ Osteopontin is produced at high levels by the macrophages found in granulomas of diverse etiology, including those induced by Mycobacterium tuberculosis,^{(19,20)} consistent with its having an anti-inflammatory role. An anti-infectious role has long been suspected because of its association with resistance to certain infectious agents.⁽⁷⁾ OPN also induces cellular chemotaxis and haptotaxis,^{(21,22)} and it stimulates the infiltration of monocytes and macrophages to sites of subcutaneous OPN injection,⁽²³⁾ possibly through a mechanism involving CD44.⁽²⁴⁾ There is a strong association between enhanced OPN expression and monocyte/macrophage infiltration at sites of focal injury in the kidney.⁽²⁵⁻²⁷⁾

Despite the variety of activities attributed to OPN, and its prominence in many normal and pathological tissues, its significance to the vertebrate organism remains to be elucidated. It is frequently found in pathological calcifications such as atherosclerotic plaques,⁽²⁾ sclerotic glomeruli,⁽²⁸⁾ and kidney stones.^{(29,30)} Its high expression in osteogenic cells and its accumulation in the calcified extracellular matrices of bone and teeth have been well established, seemingly implicating OPN in the development and remodeling processes of mineralized tissues.⁽³⁾

Its presence at mineralized tissue surfaces and interfaces⁽³¹⁾ and its facilitation of phagocytosis of OPN-coated particulates are consistent with a role in promoting cell attachment and removal of foreign bodies.⁽³²⁾ Its prominent distribution throughout bone, and in particular its concentration at cement lines, has prompted the suggestion that OPN participates in hard tissue cohesion and may promote interfacial adhesion between apposing substrata.^{(31,33)} Other in vitro studies have identified OPN as a potent inhibitor of hydroxyapatite (calcium phosphate) crystal formation and growth.^{(33,34)}.

The precise roles of osteopontin in normal tissue development and maintenance, as well as in embryogenesis and fetal development are not known at this time. Due to the putative biological importance of osteopontin in bone formation and cell attachment, the osteopontin gene is an important target for embryonic stem cell manipulation.

The generation of osteopontin deficient-transgenic mice would aid in defining the normal role(s) of osteopontin and facilitate the use of an animal model of osteopontin deficiency in the design and assessment of chemical approaches to inhibiting or augmenting osteopontin activity. Such osteopontin modified transgenic mice may also be as a source of cells for cell culture.

### SUMMARY OF THE INVENTION

This invention provides non-human transgenic animals in which the osteopontin gene has been altered and methods of use thereof. The osteopontin knockout mice of the invention are fertile and develop normally.

Osteopontin plays a role in numerous physiological processes. Osteopontin-related processes include, but are not limited to, bone remodeling, angiogenesis, inhibition of nitric oxide production, renal pathologies, atherosclerosis, monocyte differentiation, osteoporosis and osteoclast function. However, the molecular mechanisms by which osteopontin effectuates these processes have yet to be elucidated.

In a preferred embodiment of the invention, mice transgenic for the osteopontin gene are provided. Such mice may be used to advantage in assays for the identification of therapeutic agents useful for the treatment of osteopontin related pathologies.

In accordance with one aspect of the present invention, it has been discovered that osteopontin knockout mice are resistant to ovariectomized-induced osteoporosis. Thus, these mice may be used to advantage to screen therapeutic agents that inhibit or promote osteoporosis.

In yet another aspect of the invention, it has been discovered that osteopontin-deficient mice are more susceptible to ischemic damage of the kidney than are wild-type mice. Accordingly, methods are provided for assessing therapeutic agents for the treatment such renal disorders.

Osteopontin is a highly conserved plasma protein. While antibodies to the protein exist, antibodies specific for all of the epitopes on the protein are difficult to obtain as these highly conserved regions will not be recognized as “non-self” following antigenic stimulation. The osteopontin knock-out mice of the invention are used in methods for the development of osteopontin-specific monoclonal antibodies. Use of the knock out mice described herein should provide a superior array of antibodies specific for osteopontin.

Thus, the invention also comprises at least one monoclonal antibody which recognizes at least one human osteopontin epitope shown in FIG. 29.

Also provided is a method for modulating autoimmune disease in a patient in need thereof, comprising administration of an effective amount of at least one monoclonal antibody immunologically specific for osteopontin, said administration being effective to reduce osteopontin levels. In a preferred embodiment, the antibody binds the amino terminus of osteopontin.

In yet another aspect, a method for modulating corticosteroid levels in a patient in need thereof is disclosed, comprising administration of an effective amount of at least one anti-osteopontin monoclonal antibody in a biologically suitable carrier.

### BRIEF DESCRIPTION OF THE DRAWINGS

FIG. 1 is a map of the Opn on locus and the targeting construct used to create the transgenic mice of the invention. The targeting construct is depicted above the genetic map. Open boxes are exons. The stippled box is the promoter element in the neo cassette, and the open box labeled oen is the neomycin phosphotransferase gene. Dashed lines indicate where the ends of the targeting construct fall in the Opn gene. Selected restriction sites are indicated: H=HincII; E=EcoRI; B=BamHI; Bx=BstXI; Ea=EagI, H3=Hind III, A=sequence used as a probe for osteopontin. The sizes of the expected HincII fragments are indicated.

FIG. 2 is a blot showing the results of Southern analysis of DNA from a targeted cell line and from two mice. Genomic DNA was prepared from cells or tail DNA and digested with HincII. The fragments were separated and hybridized to the probe indicated in FIG. 1 (hatched box labeled A) which hybridizes to a region of the Opn gene that is outside the region of homology between the Opn gene and the targeting allele. The positions of the wildtype (WT) and disrupted (DIS) alleles are indicated. Lane 1 is DNA from the parental, wildtype AB2.1 cell line; lane 2 is DNA from the targeted 9B cell line; lane 3 is DNA from a mouse heterozygous for the Opn disruption; and lane 4 is DNA from a mouse homozygous for the Opn disruption.

FIGS. 3A, 3B and 3C are Northern and Western blots showing the absence of OPN Expression in Opn^{-/-} Mice. The results of Northern analysis of kidney RNA prepared from mice with different Opn alleles are shown in FIG. 3A. Total RNA was prepared from kidneys of mice of different genotypes and fractionated on an agarose gel. The resulting blot was probed with a fragment of the Opn cDNA extending from the 5′ end of the RNA to the EagI site in exon 6. Lane 1: +/+, 5 μg; lane 2: +/−, 5 μg; lane 3: +/−, 0.5 μg; lane 4: +/−, 0.1 μg; lane 5: −/−, 5 μg; lane 6: −/−, 20 μg. Identical results were obtained with a probe representing Opn sequences 3′ of the EagI site. FIG. 3B shows the results of Western blot analysis of OPN protein in various tissues. Protein samples were separated on 12% SDS polyacrylamide gels, and transferred to Immobilon-P membranes. These blots were incubated with goat anti-rat OPN IgG (OP-199, lanes labeled 199) or with control IgG (lanes labeled nIgG), and visualized by enhanced chemiluminescence. Lane 1: 4 μL of medium conditioned by RAW264.7 cells; lanes 2-5: CM—concentrated medium conditioned by primary mouse embryo fibroblast cells, 20 μg protein/lane; lanes 6-9: 10 μl of undiluted mouse urine; and lanes 10-13: 5 μg bone extract protein. OPN from bone migrates more rapidly on this gel than do the other forms of OPN, possibly because of lower phosphate content. Smearing at the top of the bone +/+ lane (10) incubated with anti-OPN probably represents high molecular weight aggregates of OPN (67). FIG. 3C shows the presence and relative concentration of cross reacting fragment in −/− bones. Protein extracts from +/+ and −/− bones were fractionated on a 12% SDS-polyacrylamide gel. Left panel: Lane 1: +/+ bone extract, 0.5 μg; lane 2: +/+ bone extract, 0.05 μg; lane 3: +/+ bone extract, 0.01 μg; lane 4: −/− bone extract, 5 μg. This blot was reacted with antiserum 199 as described above. Right panel: Lane 1: +/+ bone extract, 0.5 μg; lane 2: +/+ bone extract, 0.05 μg; lane 3: −/− bone extract, 5 μg; reaction was with antiserum 732. Positions of molecular weight markers (in kD) are shown, and the position of wt OPN is indicated (OPN). The arrows indicate the position of the cross reacting 35-kD species. Antiserum 732 to mouse OPN was made in the Opn^{-/-} mice (Kowalski et al., unpublished data) so that the secondary antibodies used also detect endogenous mouse IgG; the position of these bands in the right panel is indicated by dots.

FIGS. 4A and 4B are a pair of micrographs showing the histology of the proximal tibial growth plate in Opn^{+/+} and Opn^{-/-} mice. Light microscopic features of both wildtype (FIG. 4A, Opn^{+/+}) and mutant (FIG. 4B, Opn^{-/-}) tissues are similar in that the growth plates (GP) subjacent to epiphyseal bone (EB) contain columns of chondrocytes that typically proceed through proliferative and hypertrophic stages. In mice of both genotypes, bone (B) is deposited by osteoblasts onto spicules of calcified cartilage (C) to form the primary spongiosa (PS). These are epoxy resin (Epon) sections obtained from decalcified specimens and stained with toluidine blue.

FIGS. 5A-5D are micrographs showing bone ultrastructure and immunocytochemistry in Opn^{+/-} (FIG. 5B) and Opn^{-/-} (FIGS. 5 A,C and D) animals. As observed here by transmission electron microscopy of undecalcified samples of tibia from mutant mice, and as similarly noted for wildtype animals, bone-forming osteoblasts (Ob) secrete a layer of generally unmineralized osteoid matrix (OS) that subsequently calcifies to become the mineralized matrix (MM) proper of bone. As for normal bone, calcification commences as small foci within the osteoid (arrows), with mineral confluence being achieved at the interface between the osteoid and the mineralized matrix—the so called mineralization front. Osteoblast lineage cells become trapped in the matrix and are identified as osteocytes (Oc). See FIG. 5A.

FIG. 5B shows the results of post-embedding, colloidal-gold immunocytochemistry for OPN in heterozygous (illustrated here) and wildtype mice. The results reveal immunolabeling throughout the bone matrix, particularly in cement lines (CL).

FIG. 5C shows the results of immunocytochemistry performed as in FIG. 5B on sections of bone from OPN −/− mice. The absence of colloidal-gold particles over cement lines confirms the lack of OPN in these structures. FIG. 5D shows micrographs of bone matrix immunolabeled for BSP in Opn^{-/-} mice. The data show an otherwise normal distribution of gold particles throughout the bone and also at cement lines (CL). FIG. 5A, Epon section of undecalcified tibia stained with uranyl acetate and lead citrate. FIGS. 5B-D, LR White sections of decalcified alveolar bone from the mandible immunolabeled for OPN or BSP and counterstained with uranyl and lead.

FIGS. 6A-6E show tartrate-resistant acid phosphatase staining of osteoclasts developing in cultures with ddy osteoblasts⁽⁴⁷⁾ as described in methods. FIG. 6A, +/+; FIG. 6B, +/−; FIG. 6C, −/−; FIG. 6D, +/+; FIG. 6E, −/−. FIGS. 6A-C: osteoclasts developed from spleen precursors; FIGS. 6D-E osteoclasts from bone marrow precursors. Original magnification ×40.

FIG. 7 is an immunoblot showing osteopontin expression in ddy osteoblast cultures. Osteoblasts were prepared from ddy calvaria, and cultured for 8 days. At the end of the culture period the cells were incubated in serum-free medium for an additional 1 day (1d CM), 2 days (2d CM), or 3 days (3d CM) as indicated above the lanes, and this conditioned medium was collected. 15 μl of these conditioned media were fractionated directly on an SDS polyacrylamide gel, transferred to Immobilo-P and reacted with OP-199 IgG as described in the legend to FIG. 3B and Materials and Methods.

FIGS. 8A-8D show micro-CT analysis of the tibiae of wild-type and osteopontin deficient mice. Wild type (FIGS. 8A, 8B, 8E, 8F) or osteopontin-deficient (FIGS. 8C, 8D, 8G, 8H) mice were either ovariectomized (FIGS. 8A, 8C, 8E, 8G) or sham-operated (FIGS. 8B, 8D, 8F, 8H). Four weeks postoperatively, two-dimensional micro-CT pictures of the tibiae were taken in the mid-sagittal planes as indicated by solid white lines (FIGS. 8E, 8F, 8G, 8H), by using either Musashi (Nittetsu Elex Co. Ltd) or by Scanco microCT-20 system (Scanco Co.Ltd.).

FIGS. 9A-9D show three dimensional pictures of the trabecular bone in the tibiae. Three dimensional pictures of the trabecular bones were obtained using the tibiae of the wild type (FIGS. 9A, 9B) or the osteopontin deficient (FIGS. 9C, 9D) mice which are either ovariectomized (FIGS. 9A, 9C) or sham operated (FIGS. 9B, 9D). The micro CT used was Musashi (Nittetsu Elex Co. Ltd.)

FIGS. 10A-10D show soft x-ray pictures of the tibiae. Wild type (FIGS. 10A, 10B) or osteopontin null (FIG. 100, FIG. 10D) mice were either ovariectomized (FIGS. 10A, 100) or sham-operated (FIGS. 10B, 10D). Soft X-ray pictures were taken after dissection of the tibiae. The X-ray was taken by the Softex (Model CMB-2) with exposure time for 2 seconds, and bulb voltage at 50 kV, and bulb current at 25 mA using industrial X-ray film FR type (Fuji, Tokyo)

FIGS. 11A-11D depict micrographs showing histology of the tibiae of the mice. Wild type (FIGS. 11A, 11B) or osteopontin deficient (FIGS. 11C, 11D) mice were either ovariectomized (FIGS. 11A, 11C) or sham-operated (FIGS. 11B, 11D). Tibiae of the mice were subjected to histological preparation. Paraffin sections were made in the sagittal planes of the tibiae and stained with haematoxylon and eosin.

FIG. 12: Cartoon of novel biopanning protocol for antibody epitope determination using T7 phage and protein G beads. Protein G agarose beads are used to pre-clear the T7 phage library of non-specific binding phage (left). In a separate reaction, protein G beads are used to bind the antibody being assayed (right). The pre-cleared phage and the antibody-bead complexes are then incubated together to allow antibody-phage binding. The resulting complexes are washed, added directly to E. coli and plated for plaque formation. Positive plaques are identified by western blotting, and the region containing the OPN insert is amplified by PCR and sequenced to determine the peptide expressed.

FIG. 13: Localization of monoclonal anti-OPN antibody epitopes. (A) Alignment of the mouse and human OPN sequences showing the determined epitopes of our monoclonal antibodies. Antibody recognition sites are underlined with the antibody name below. Posttranslational modifications of human OPN are taken from Christensen et al. (2005). Phosphorylated residues are highlighted in gray. Glycosylated residues are written in grey. (B) Peptides resulting from phage display screening demonstrating the determination of the epitope for antibody AK2A1.

FIG. 14: Monoclonal antibody recognition of OPN. (A) Western blotting results showing antibody recognition of murine OPN. Conditioned media (IOplIlane) from various cell lines or 50 ng of recombinant murine OPN (GST-mOPN) were separated on 12% SDS-PAGE gels and transferred to PVDF membranes. The membranes were then cut into strips which were blotted with monoclonal antibodies at 1 μg/ml or polyclonal control at a 1:3000 dilution (shown above each lane). 275-3-2: ras-tranformed murine fibroblast cell line. 275: non-transformed murine fibroblast 3T3 cell line. MC3T3E1: pre-osteoblast cell line induced to differentiate for 12 days as described in materials & methods. (B) Western blot of human urine detected with anti-OPN monoclonal antibodies. Urine was collected and dialyzed extensively against 0.1M NaCl before approximately 10-fold concentration with Centriprep spin columns. Five μl of the concentrated, dialyzed urine was assayed via SDS-PAGE and western blotting with monoclonal antibodies AK1H3, AK3D9, AK10F6, AK2A1, and AK2C5 at 1 μg/ml. Polyclonal antibody LF 124 (kindly provided by Dr. Larry Fisher, NIH) and polyclonal anti-OPN (recombinant) mouse serum were used at 1:750 and 1:3000 respectively.

FIG. 15: Phosphorylation blocks 3D9 binding. (A) Peptides used for the antibody-peptide binding assay kindly provided by Dr. Larry Steinman. (B) Binding of antibodies AK3D9 and AK7B4 to synthetic human OPN peptides. The biotinylated peptides were coated onto Neutraavidin plates at 10 μg/ml and detected with 5 μg/ml AK3D9 or AK7B4 monoclonal antibody following the manufacturer's instructions (Pierce Biotech). The secondary antibody used was Alexafluor 594 goat anti-mouse IgG (Molecular Probes) and fluorescence was detected using excitation/emission wavelengths of 58416 12 nm. Data shown are representative of three independent experiments (n=21exp).

FIG. 16: Antibody inhibition of cell adhesion to recombinant human OPN. Tissue culture treated 96-well plates were coated with 150 μM human recombinant his-tagged OPN, then blocked with 1% BSA. Antibodies were then added at 125 μM and allowed to bind OPN for 2 hr. The wells were then washed and 5×10˜M DA-MB-435 (A) or 275-3-2 (B) cells were added and allowed to adhere for 3 or 3.5 hr respectively. Non-adherent cells were removed by washing and adherent cells were quantitated by staining with crystal violet. Data are representative of 4 independent experiments for the MDA-MB-435 cell line and 2 independent experiments for the 275-3-2 cell line (n=4). *, p<0.001 Student's t test.

FIG. 17: CRS-induced organ atrophy in 129 and Balb/c mice. (A) Representative data showing thymus and spleen weight change in 129 mice after 3 cycles of restraint (n ˜6). (B) Representative data showing thymus and spleen weight reduction in Balb/c mice after 2 cycles of restraint (n=6-9, combined data from three independent experiments). Data represent means±SEM. Statistical difference between OPN^{+/+} and OPN^{-/-} mice shown as *=p<0.05, **=p<0.01 with student's t test in Excel software.

FIG. 18: Changes in hormone levels in response to CRS. (A) Corticosterone levels in plasma of CRS-treated mice. Blood samples from OPN^{+/+} and OPN^{-/-} Balb/c mice were harvested immediately after the termination of CRS and plasma samples were isolated and stored at −80° C. until assay. CORT assay was conducted with plasma samples diluted 40-fold and incubated in a plate pre-coated with anti-corticosterone antibody. Data represent mean±SEM of 5-7 samples. (B) Plasma ACTH Level in 129 OPN^{-/-} Mice. ACTH levels in plasma of WT and KO 129 mice before and after CRS were measured with an ACTH ELISA kit. Data represent mean±SEM of 5-7 samples. Data represent means±SEM. Statistical difference between control and CRS-treated OPN^{+/+} and OPN^{-/-} mice shown as *=p<0.05, ***=p<0.001 with student's t test in Excel software.

FIG. 19: Effect of CRS on lymphocyte populations in blood and thymus. Immune cells harvested from blood, spleen and thymus were stained with antibodies for CD4 (CD4⁺T helper cells), CD8 (CD8⁺ cytotoxic T cells) and B220 (B cells) conjugated with fluoro-cytochromes. Percentages of each cell population were quantified by flow cytometry. Representative dot plots of each organ examined were presented as A=WT control, B=WT CRS-treated, C=KO control, D=KO CRS-treated. Numbers in each quadrant indicated the percent (%) of the specific populations in total lymphocytes. Data summarizing all animals in the treatment groups (n=4-5) are presented in Table 1.

FIG. 20: Response of Balb/c OPN^{-/-} mice to exogenous OPN. OPN^{-/-} Balb/c mice were injected daily with purified OPN (5 μg/mouse) 3 days before and 2 days during CRS. Animals were divided into three groups: Control group (n=6), untreated; CRS group (n=6) was injected with PBS and restrained; CRS+OPN group (n=6) was injected with OPN in PBS and restrained. Wild type littermates were treated in parallel for comparison with 3 animals in control group and 5 animals in CRS group. Data represent mean±SEM. Statistical significance indicated as ns=not significant, p>0.05; *=p<0.05; **=p<0.01; and ***=p<0.001. Value generated by student t test in Excel software.

FIG. 21: OPN levels in plasma of 129 OPN^{-/-} mice after injection of OPN. Plasma samples were harvested from 129 mice after CRS. OPN levels were assayed by ELISA using OPN antibodies from R&D Systems. OPN concentrations were calculated with mouse recombinant OPN from R&D Systems as a standard. Assays were conducted on samples from multiple experiments stored at −80° C. Data represent mean±SEM (n=4-7).

FIG. 22: Approximate locations of epitopes recognized by mAbs. Representation of the structure of the OPN protein with mAb binding regions indicated (18, 22 Kowalski, 2005; Kazanecki et al., 2007). mAbs 1G4 and 3D9 recognize the extreme N- and C-terminal regions respectively; 2A1 recognizes a region in the middle of the C terminal half of OPN and 2C5 recognizes a region upstream of the RGD sequence that is important for integrin interaction.

FIG. 23: Effect of 4 different anti-OPN mAbs on CRS-induced thymus atrophy in wild type mice. In 4 independent experiments, OPN^{+/+} mice in a Balb/c or 129 background were injected with 4 different anti-OPN mAbs (100 μg) 24 h before CRS and immediately prior to each cycle of restraint. Animals were divided into three groups: Control group (n=2-4), untreated; CRS group (n=5-6), injected with PBS and restrained; CRS+mAb group (n=5-7) was injected with OPN in PBS and restrained. At the end of the CRS session (two 24-h-cycles for Balb/c mice, three 24-h-cycles for 129 mice), the spleens and thymuses were evaluated for loss of weight compared to the control group. Spleen data are not shown here because CRS caused insignificant changes in spleen weight in all 4 experiments. Data represent mean±SEM. The statistical significance indicated by the p value was generated by the Student t test in Excel software.

FIG. 24: CORT levels under different conditions. (A) Plasma CORT levels in Balb/c OPN^{-/-} mice subjected to CRS and OPN injection. Plasma harvested immediately after termination of CRS was assayed using a CORT ELISA. The assay was conducted with plasma samples diluted 10-fold and incubated in a plate pre-coated with anti-corticosterone antibody. Data represent mean±SEM of 5 replicates in each group. (B) Plasma CORT levels in 129 OPN^{+/+} mice subjected to CRS and 2C5 injection. Plasma harvested immediately after termination of CRS was assayed using a CORT ELISA. The assay was conducted with plasma samples diluted 10-fold and incubated in a plate pre-coated with anti-corticosterone antibody. Data represent mean±SEM of 5 replicates in each group.

FIG. 25: Schematic diagram of OPN-induced survival of T cell. OPN induces phosphorylation and retention in cytosol of FoxO3a. NF-kB activation is also induced by OPN. The inhibition of FoxO3a along with activation of NF-kB results in induction of pro-survival proteins. The expression of anti-survival Bcl-2 family proteins, Bim, Bak and Bax is altered by OPN. Translocation of AIF to nucleus from mitochondria, where AIF plays role as a pro-survival protein, is inhibited by OPN.

FIG. 26: OPN Modulates IL-17. IL-17 is upregulated in mouse CD4 T cells reactive to MOG 35-55 with addition of r-OPN to the cultures. An anti-OPN mab lowers IL-17 production MOG TCR Tg (2D2) naïve Lymph node cells were isolated and were cultured with MOG p35-55 (10 μg/ml) in the presence of mouse recombinant OPN (2, 5 or 10 μg/ml) (R&D Systems) or 10 □g/ml anti-OPN antibody (R&D Systems) for 48 h. IL-17A was measured from supernatants using ELISA (R&D Systems Kit #DY421E).

FIG. 27: In Human T Lymphocytes, antibodies to a4b1 integrin modulate IL-17A production. In the upper 2 panels we show IL-17 and IFN-γ secretion of CD4⁺ cells from donor #1 treated with antiCD3/CD28 treated with 2A1, the same anti-OPN mab that diminishes paralysis in EAE in FIG. 10, versus isotype control. Panels below in color 8B, show the effect of rOPN on driving TH1 and TH17 cytokines and their inhibition with some antiVLA4 and CD44 mabs.

FIG. 28: Attenuation of EAE after onset with anti-OPN 2A1 given 200 micrograms on the days indicated. The antibody 10f6 was ineffective. *<0.05 via Mann-Whitney on days indicated.

FIG. 29: Further identification of location of the epitopes of certain of the mAbs in the human OPN molecule. Shown are 20 overlapping peptides (overlaps are boxed), along with known sites of O-liked glycosylation (blue) and potential serine phosphorylation (red).

FIG. 30: Recombinant OPN induced splenocytes migration. Splenocytes seeded in the transwells were incubated with recombinant mouse OPN(R&D Systems) at various concentrations in the lower chambers for 3 h. MIP-3 was used as a positive control at 2 μg/ml. Cells migrated to the lower chambers were harvested and enumerated on FACSCalibur for 30 sec. All measurements were conducted in duplicate.

FIG. 31: Chemotaxis assay with OPN fragments. Splenocytes seeded in transwells were incubated for 3 h with OPN fragments including “AKDK”: AA 205-262, “C18”:AA 1-145/147, “SKK”: AA 148-204, “SP200”: mixture of two N-terminal variants. All fragments were used at 4 μg/ml in the lower chamber. Cells migrated to the lower chambers were harvested and enumerated on FACSCalibur for 30 sec. All measurements were conducted in duplicates.

### DETAILED DESCRIPTION OF THE INVENTION

Osteopontin is an arginine-glycine-aspartate (RGD) containing glycoprotein encoded by the gene secreted phosphoprotein 1 (spp1). ssp1 is expressed during embryogenesis, wound healing, bone remodeling, and tumorigenesis. Osteopontin is involved in a variety of additional physiological processes, including angiogenesis, osteoclast function and osteoporosis. To further understand the role osteopontin plays in these processes, transgenic animals are generated which have an altered osteopontin gene. The alterations to the osteopontin gene are modifications, deletions, and substitutions. Modifications and deletions render the naturally occurring gene nonfunctional, producing a “knock out” animal. Substitutions of the naturally occurring gene for a gene from a second species results in an animal which produces an osteopontin gene from the second species. Substitution of the naturally occurring gene for a gene having a mutation results in an animal with a mutated osteopontin protein. A transgenic mouse carrying the human osteopontin gene is generated by direct replacement of the mouse osteopontin gene with the human gene. These transgenic animals are critical for drug antagonist studies on animal models for human diseases and for eventual treatment of disorders or diseases associated with cellular activities modulated by osteopontin. A transgenic animal carrying a “knock out” of osteopontin is useful for the establishment of a nonhuman model for diseases involving osteopontin regulation.

As a means to define the role that OPN plays in mammalian systems, mice have been generated that cannot make OPN because of a targeted mutational disruption of the OPn gene. These mice develop normally and are fertile. Although no histologically detectable phenotype is apparent in the bones and teeth of mice lacking OPN, the frequency with which spleen and bone marrow cells from Opn−/− mice form osteoclasts in in vitro co-cultures is elevated in comparison with cells from Opn^{+/+} mice.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring in fact, possess some or all of that alteration or genetic information, then they, too, are transgenic animals.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene.

The altered osteopontin gene generally should not fully encode the same osteopontin protein native to the host animal and its expression product should be altered to a minor or great degree, or absent altogether. However, it is conceivable that a more modestly modified osteopontin gene will fall within the compass of the present invention if it is a specific alteration.

The DNA used for altering a target gene may be obtained by a wide variety of techniques that include, but are not limited to, isolation from genomic sources, preparation of cDNAs from isolated mRNA templates, direct synthesis, or a combination thereof.

A type of target cell for transgene introduction is the embryonal stem cell (ES). ES cells may be obtained from pre-implantation embryos cultured in vitro.⁽⁶⁸⁻⁷⁰⁾ (Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated osteopontin genes to selectively inactivate the wild-type gene in totipotent ES cells (such as those described above) and then generate transgenic mice. The use of gene-targeted ES cells in the generation of gene-targeted transgenic mice was described, and is reviewed elsewhere⁽⁷¹⁻⁷²⁾.

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones.

Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodouracil, (FIAU). By this counter selection, the fraction of homologous recombinants in the surviving transformants can be increased.

As used herein, a “targeted gene” or “knock-out” is a DNA sequence introduced into the germline or a non-human animal by way of human intervention, including but not limited to, the methods described herein. The targeted genes of the invention include DNA sequences which are designed to specifically alter cognate endogenous alleles.

Methods of use for the transgenic mice of the invention are also provided herein. Such mice may be used to advantage to identify agents which augment, inhibit or modify the activities of osteopontin. For example, osteopontin knock out mice are resistant to ovariectomized induced osteoporosis. Accordingly, therapeutic agents for the treatment or prevention of osteoporosis may be screened in studies using ovariectomized and non-ovariectomized osteopontin knock out mice. For example, osteopontin knockout mice may be treated with a test compound that induces osteoporosis. Secondary reagents could then be assessed which inhibit or suppress the osteoporotic pathway. Such assays will not only facilitate the identification of agents which regulate osteoporosis, they should also be illustrative of the underlying biochemical mechanisms which underlie the disorder.

Osteopontin knockout mice are also more susceptible to ischemia induced renal damage. Thus in another embodiment of the invention, ischemia of the kidney is induced in osteopontin deficient and wild type mice by clamping the renal artery to prevent blood flow to the kidney. After 30 minutes the clamps are removed and kidney tissue assessed for damage. This damage may be quantified by measuring the levels of blood urea nitrogen and creatinine following reperfusion of the ischemic kidneys. These parameters have been shown to be about two-fold higher in osteopontin deficient animals when compared to wild type controls.

Osteopontin also plays a role in inhibiting formation of nitric oxide. The levels of inducible nitric oxide synthase and nitrotyrosine, an indicator of nitric oxide levels in vivo, were dramatically elevated in post-ischemic, osteopontin deficient kidneys as compared with the post-ischemic wild-type kidneys. These results implicate osteopontin in protecting the kidney against ischemia-induced damage via a mechanism involving a reduction in nitric oxide production. The data also provide evidence that, in vivo, osteopontin is instrumental in reducing inducible nitric oxide synthase confirming results observed in vitro.

In another embodiment of the invention, osteopontin knockout mice are used to produce an array of monoclonal antibodies specific for osteopontin. Antibodies so produced are also described which should have efficacy for the treatment of autoimmune disease as OPN is known to promote the progression of autoimmune diseases (e.g. EAE, RA) in the mouse. OPN exists in various isoforms with differing post-translational modifications.

Differences in PTMs influence the functional behavior of OPN in both physiological and pathophysiological processes such as cell migration, cell adhesion, and cell proliferation. The ability to inactivate functionally specific isoforms of OPN with particular mAbs can provide an essential step in modulating OPN's in vivo actions.

Osteopontin (OPN) is also a cytokine implicated in mediating responses to certain stressors, including mechanical, oxidative and cellular stress. The present inventors have determined that that injection of OPN into the OPN-deficient mice described herein enhances CRS-induced lymphoid organ atrophy and that injection of a specific anti-OPN monoclonal antibody (2C5) into wild type mice ameliorates the CRS-induced organ atrophy; changes in corticosterone levels were also partially reversed. These studies reveal that OPN plays a significant role in the regulation of the hypothalamus-pituitary-adrenal axis hormones and that it augments CRS-induced organ atrophy. This observation provides novel methods for identifying therapeutic agents which modulate this process.

The following methods are provided to facilitate the practice of Example I and II.

### Generation of Opn−/− Mice

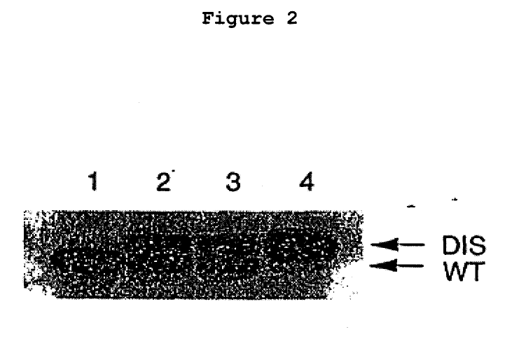

Osteopontin genomic clones were obtained from a mouse strain 129 genomic library (a generous gift from F. Alt) by screening with a fragment of the Balb/c Opn gene.⁽³⁷⁾ Positive clones were mapped and a 4.8-kb BamHI-HindIII fragment subcloned into pBluescript. The targeting construct was made by inserting the neo cassette from pMC1 neo⁽³⁸⁾ into this plasmid at the unique EagI site in exon 6, in the reverse orientation relative to OPN transcription. A thymidine kinase cassette from pMC1TK1⁽³⁹⁾ was inserted just 3′ of the Opn sequences, in the reverse transcriptional orientation. This construct was linearized with BamHI and 100 μg of purified DNA electroporated into 4×10⁸ AB2.1 cells.⁽⁴⁰⁾ Transfected cells were plated onto mitomycin-C treated SNL-767 fibroblasts, and drug-resistant cells were selected in G418 plus gancyclovir. Surviving clones were placed into 96-well plates and expanded. Correctly targeted clones were identified by PCR and confirmed by southern blotting as shown in FIG. 2. Cells from two clones that had undergone the desired recombination event were injected into C57B1/6 blastocysts, which were then implanted into pseudopregnant CD-1 female mice. One of the two clones gave germline transmission of the ES cell phenotype. Genomic DNA from cells or mouse tail fragments was isolated by proteinase K digestion, extracted with phenol, and precipitated with ethanol. Chimeric males were mated to C57B1/6 females, and the subsequent heterozygous F1 animals were crossed to generate Opn^{+/+} and Opn^{-/-} lines. All animal studies were conducted using protocols approved by the Rutgers Institutional Review Board for the Use and Care of Animals.

Analysis of OPN mRNA and Protein

RNA was prepared by using TriReagent (GibcoBRL, Gaithersburg, Md.). Total cellular RNA was fractionated on 1% agarose gels in the presence of formaldehyde and transferred to Gene Screen Plus (Dupont NEN, Boston, Mass.). These blots were hybridized at 42° C. overnight in the presence of 50% formamide. Western blotting was used to detect OPN in various tissues and body fluids. Serum-free Dulbecco's minimal essential medium, conditioned by mouse embryo fibroblasts for 16 hr, was concentrated about 50-fold prior to analysis. Urine was not concentrated. Protein was extracted from bones as described.⁽⁴¹⁾ Briefly, bones were flash frozen in liquid N₂, pulverized, and extracted with 4 M guanidine-HCl in 50 mM Tris-HCl, pH 7.3. This extract was discarded, and the residue was further extracted with 4 M guanidine-HCl in 50 mM Tris-HCl, pH 7.3, containing 0.5 M Na₂EDTA, twice for 24 hr each time. The EDTA extracts were combined and the buffer was changed to 6 M urea in 50 mM Tris-HCl, pH 7.3. Proteins were extracted from kidney and lactating mammary glands in RIPA buffer as previously described.⁽⁴²⁾ Protein concentration was determined by using the bicinchoninic acid assay (Pierce Chemical, Rockford, Ill.). Proteins were separated on 12% SDS-polyacrylamide gels and transferred to Immobilon-P membranes (Millipore, Bedford, Mass.). These blots were blocked with 1% nonfat dry milk and reacted with the indicated antibody preparations. Antibody reactivity was visualized with enhanced chemiluminescence (Amersham, Chicago, Ill.).

### Antibodies

Goat anti-rat OPN antiserum 199⁽²¹⁾ was kindly provided by Dr. Cecilia Giachelli, and was used in westerns at a dilution of 1:1500, and in immunocytochemistry at a dilution of 1:10. Antiserum 732 is a mouse anti-mouse OPN polyclonal serum developed in our laboratory in the Opn −/− mice (Kowalski et al., unpublished data), and was used in westerns at a dilution of 1:1500 or less. Antiserum to bone sialoprotein (BSP) was LF-6, kindly provided by Dr. Larry Fisher⁽⁴³⁾.

### Bone Histology and Immunocytochemistry

Mandibles, tibiae and calvariae from 2-4 month old mice were fixed in 0.1 M sodium cacodylate-buffered 4% paraformaldehyde/1% glutaraldehyde and analyzed as described.⁽³¹⁾ Briefly, bones were left undecalcified or were decalcified for two weeks in 4% disodium EDTA, dehydrated and embedded in Epon or LR White acrylic resin. One-micrometer-thick sections were cut and stained with von Kossa reagent or with toluidine blue for light microscopy; 80-100 nm sections on nickel grids were used for ultrastructural analyses by transmission electron microscopy and for colloidal-gold immunocytochemistry. Post-embedding immunolabeling for OPN⁽⁴⁴⁾ was performed using the antibody OP-199⁽²¹⁾, and for bone sialoprotein (BSP) using the antibody LF-6 followed by protein A-gold (10-14 nm diameter gold particles) and conventional staining with uranyl acetate and lead citrate. Incubation of sections with preimmune serum, irrelevant polyclonal antibody, or protein A-gold alone served as controls.

Morphological observations and immunocytochemical labeling patterns were recorded using a Zeiss Axiophot light microscope and a JEOL TEM 2000 FX II electron microscope operated at 80 kV.

### Osteoclast Formation in Vitro

Osteoblast cultures were prepared from calvariae of neonatal mice of the indicated strain by sequential collagenase digestion as described⁽⁴⁵⁾ and maintained in a-minimal essential medium (MEM) with 10% fetal calf serum (GibcoBRL, Grand Island, N.Y.). Bone marrow cells were obtained by flushing the cells from the medullary cavity of femurs with α-MEM. The dispersed cells were washed, counted, and 2.5×10⁵ cells/cm² plated on 1×10⁴ osteoblasts in 24-well plates. Similarly, 1×10⁵ spleen cells, obtained as described⁽⁴⁶⁾ were plated on osteoblasts in 24-well plates. These cultures were maintained in α-MEM in 10% fetal calf serum in the presence of 10⁻⁸ M 1α, 25-dihydroxyvitamin D₃ for seven days. Osteoclasts were identified by staining for tartrate-resistant acid phosphatase and classified according to the number of nuclei.⁽⁴⁷⁾

The following Examples are provided to illustrate various embodiments of the invention. They are not intended to limit the invention in any way.

### Example I

### Generation and Characterization of OPN−/− Knock Out Mice

Homologous recombination in embryonic stem cells has been utilized to generate mice with a targeted disruption of the osteopontin (Opn, or Spp1, for secreted phosphoprotein 1) gene. Mice homozygous for this disruption fail to express OPN as assessed at both the mRNA and protein level, although an N-terminal fragment of OPN is detectable at extremely low levels in the bones of −/− animals. The Opn−/− mice are fertile, their litter size is normal and they develop normally. The bones and teeth of animals not expressing OPN are morphologically normal at the level of light and electron microscopy, and the skeletal structure of young animals is normal as assessed by radiography.

Ultrastructurally, proteinaceous structures normally rich in OPN, such as cement lines, persist in the bones of the Opn^{-/-} animals. Osteoclastogenesis was assessed in vitro in co-cultures with a feeder layer of calvarial osteoblast cells from wildtype mice. Spleen cells from Opn−/− mice cells formed osteoclasts 3-13 fold more frequently than did control Opn+/+ cells, while the extent of osteoclast development from Opn−/− bone marrow cells was about 2-4 fold more than from the corresponding wildtype cells. Osteoclast development occurred when Opn−/− spleen cells were differentiated in the presence of Opn−/− osteoblasts, indicating that endogenous OPN is not required for this process. These results sugges't that OPN is not essential for normal mouse development and osteogenesis, but can modulate osteoclast differentiation.

### Results

### Derivation of Opn−/− Mice

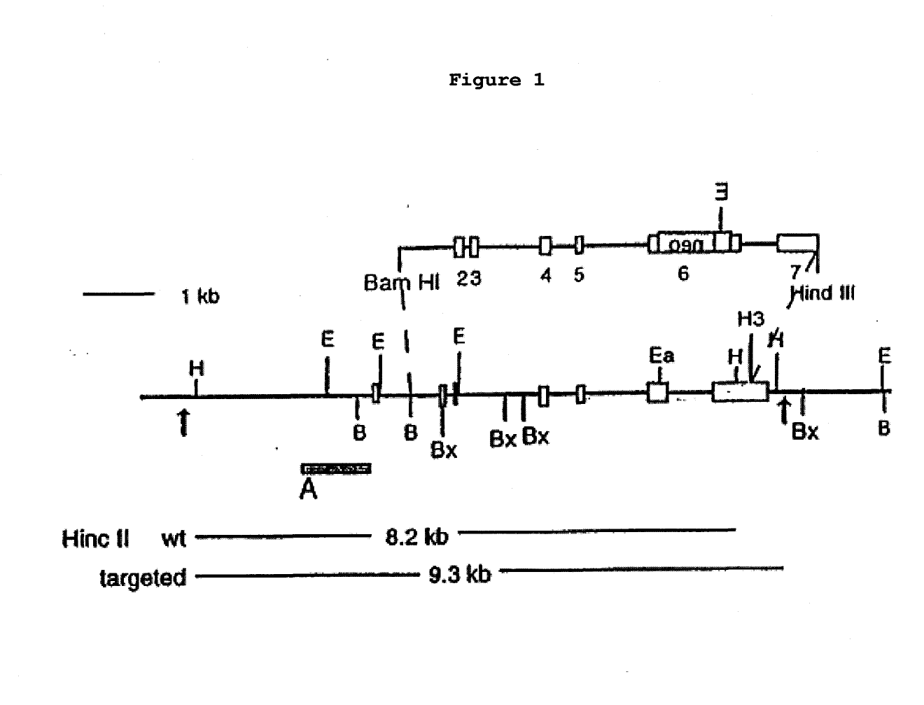

The targeting construct used to disrupt the Opn gene comprised 4.8 kB of Opn sequence from 129 strain genomic DNA containing a neo cassette inserted into the EagI site in exon 6 (FIG. 1A). This EagI site lies immediately 5′ of the RGD sequence, so that any truncated protein made from the 5′ end of the gene would lack this integrin-binding sequence. A thymidine kinase-coding sequence in the targeting vector just 3′ of the Opn sequence, and in the opposite transcriptional orientation to that of the Opn gene, allowed for enrichment of targeted clones by negative selection.⁽³⁹⁾ The linearized construct was introduced into AB2.1 embryonic stem cells by electroporation, and clones that had undergone the desired homologous recombination event were identified by PCR. The genotype was subsequently confirmed by southern analysis. Correctly targeted clones, grown in the absence of G418, were injected into C57B1/6 blastocysts. One cell line, 9B, gave rise to male chimeras that were able to transmit the disrupted Opn allele to their offspring. The resulting heterozygous F1 animals were mated to generate animals homozygous for the targeted disruption of the Opn gene, which were obtained in the expected Mendelian ratio. Southern analysis of DNA from the targeted 9B cell line and two mice containing the disrupted Opn allele confirmed that these animals were homozygous for the disrupted Opn allele (FIG. 1B).

### Assays for OPN Expression in Mice Homozygous for the Disrupted Opn Gene

To verify that OPN expression was indeed extinguished in the Opn −/− animals, we analyzed Opn mRNA and protein levels in a variety of different tissues and cell preparations (FIG. 2). The probe used in the experiment of FIG. 2A was a fragment of the Opn cDNA extending from the 5′ end of the mRNA to the Eag I site in exon 6, the site of insertion of the neo cassette in the targeting construct. This probe will hybridize to any truncated mRNA fragments which might be transcribed from the endogenous promoter in the disrupted Opn gene. No normal-sized or truncated Opn transcripts were detectable in RNA derived from kidneys of the Opn−/− mice. A higher molecular weight RNA species hybridizing with this probe was seen when large amounts of RNA from Opn−/− kidneys were analyzed (FIG. 2B, lane 6). This transcript hybridizes with both 5′ and 3′ probes, and is seen in RNA preparations from mice of both genotypes. Its identity is at present unknown.

Western blotting of a variety of tissues, fluids, and cells from these mice with the anti-OPN antiserum OP-199 confirmed that OPN protein was not detectable in the Opn^{-/-} animals (FIG. 2B). Samples for assay included medium conditioned by mouse embryo fibroblasts (lanes 2-5), urine (lanes 6-9), and an extract of bone (lanes 10-13). In many cases these results were difficult to interpret because cross reactivity of OP-199 and other antibodies was seen with several unidentified proteins, particularly in the tissue extracts. For this reason, comparisons of identical samples incubated with immune and control IgG are shown FIG. 2B. For example, in lanes 3 and 4, showing conditioned medium from embryo fibroblast cultures, several proteins migrating more rapidly than OPN in the Opn−/− sample exhibited reactivity with the 199 antiserum; however, this reactivity was also seen with the control IgG in lane 4.

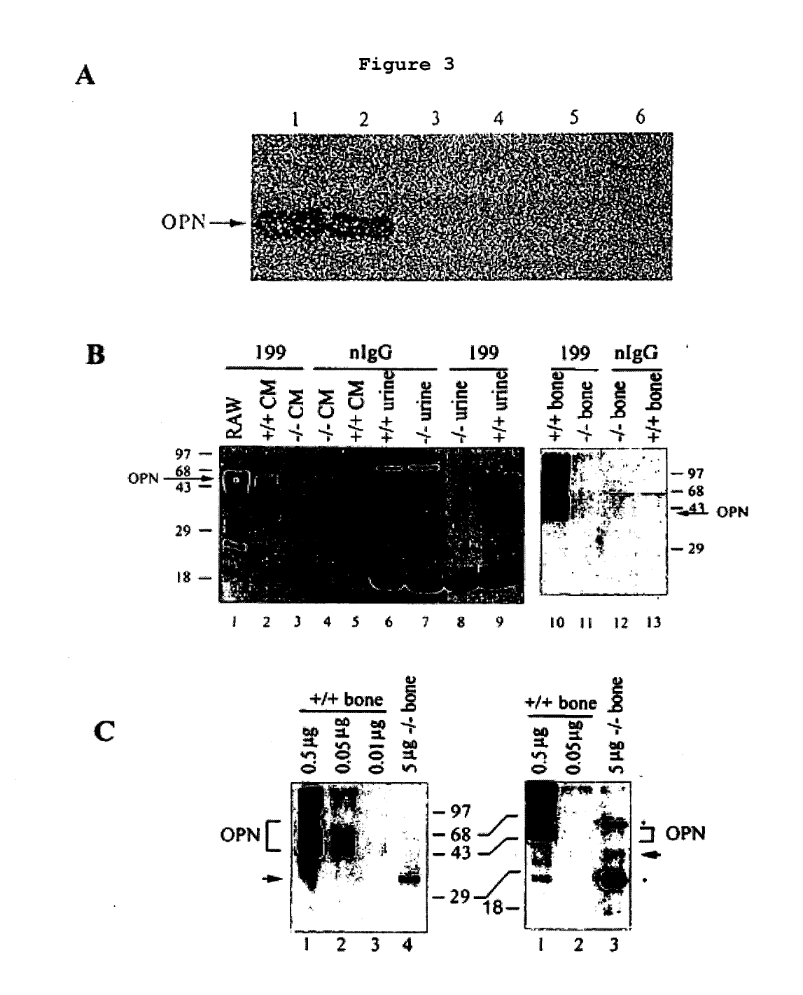

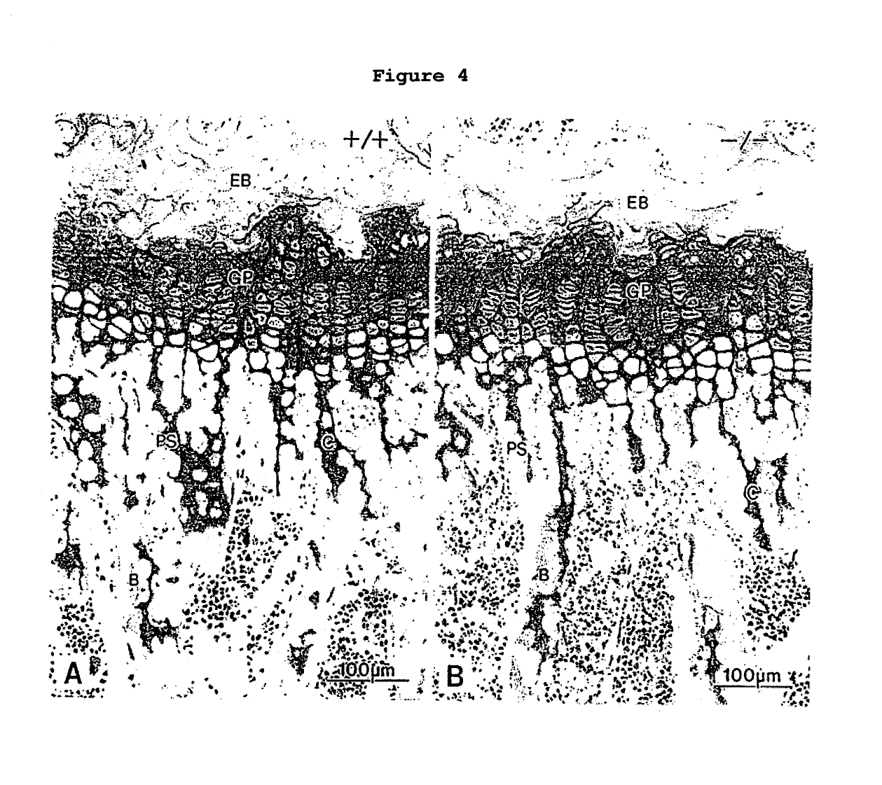

In bone extracts from the Opn −/− animals, antisera OP-199 and 732, both specific for OPN, detect a protein migrating with an apparent molecular weight of ˜35-kD in long exposures (FIG. 2C). It is likely that this protein represents a truncated form of OPN. In principle, a transcript could be generated from the endogenous OPN promoter and be completely processed to generate a 2.8-kB mRNA containing the neo sequences in exon 6. If this transcript were translated, it would give rise to an amino-terminal fragment of OPN, which would contain sequences represented in exons 2-5 and part of exon 6. Such a protein would not contain the RGD sequence, or the C-terminal half of the protein. We have estimated that the 35-kD protein is present at a level 100-200-fold lower than that of wildtype OPN, and we have been unable to detect it in any body fluids or tissues other than bone. This fragment of osteopontin would be unlikely to have any effect on the phenotype of the animals. First, it is predicted to lack the RGD sequence which has been shown to be important for OPN function in several systems. Second, while this fragment would be expected to retain the poly-Asp sequence, which might allow it to function in mineral binding, its extremely low concentration (FIG. 2C) renders it unlikely that this fragment can have any effect on the bone phenotype. Independent support for this idea comes from observations of animals with a different disruption of the OPN gene in which exons 4 through 7 are deleted⁽⁷⁸⁾. These animals lack the immunoreactive 35-kD OPN fragment, yet their bone morphology is indistinguishable from that described here (FIGS. 3 and 4, and McKee, Rittling and Liawi, unpublished data).

### Characteristics of the OPN-deficient Mice

Mice homozygous for the targeted disruption appear phenotypically normal. They are fertile and can lactate, and their litter size is normal. Weights of the animals of the different genotypes between 25 and 52 days of age do not differ significantly (data not shown). Histological examination of liver, spleen, kidney, pancreas, and lung revealed no obvious abnormalities in the Opn animals (data not shown).

### Bone Morphology in the Absence of OPN

OPN was originally isolated from bone,⁽⁴⁸⁾ and its name reflects its presumed importance in this tissue, in which it is especially abundant.⁽³⁾ We have extensively compared the bones of Opn+/+ and Opn−/− animals using radiography, light and electron microscopy, and ultrastructural immunocytochemistry. The skeletal structure of the Opn−/− animals appeared radiographically normal (data not shown). Morphologically, the cells and extracellular matrix organization and composition of the bones and teeth in the Opn−/− mice were indistinguishable from those of wildtype animals (FIG. 3 and data not shown). In bone, osteogenic cell types were readily identifiable and were present with their expected frequency and distribution. Identical results have been obtained with an independently derived strain of Opn−/− mice (McKee and Liaw, unpublished data). These results lend support to the idea that the cross-reacting 35-kD protein seen on western blots, if it is an OPN fragment, is not responsible for the lack of a phenotype in the bones of the Opn−/− mice. The disruption in the OPN gene in the mutant mice generated by Liaw and coworkers was achieved by a strategy which would not be expected to generate a similar 35-kD fragment⁽⁷⁸⁾.

Ultrastructurally, extracellular matrix organization of bone tissue in the mutant mice was unchanged, and prominent organic structures within the bone such as collagen fibrils, cement lines and laminae limitantes were all readily discernable.

Calcification of the matrix appeared unaffected by the absence of OPN. Osteoclasts with well-developed ruffled borders and otherwise normal histology were present, and numerous crenated cement (reversal) lines, indicative of bone resorption activity by these cells, were distributed throughout the bone matrix. Colloidal-gold immunocytochemistry for OPN in wildtype mice revealed intense immunolabeling of mineralized matrix in bone, tooth cementum, laminae limitantes at bone surfaces, and cement lines at sites of bone remodeling. However, in the Opn−/− mice, while normal hard tissue architecture and organization were retained (FIG. 4A), cement lines and other structural elements normally known to contain OPN (FIG. 4B) showed a complete absence of immunolabeling for this protein (FIG. 4C). Other noncollagenous extracellular matrix proteins abundant in bone, such as bone sialoprotein (FIG. 4D) and osteocalcin (data not shown), exhibited essentially normal immunolabeling patterns in the OPN-deficient mice.

### Altered Osteoclastogenesis in Vitro

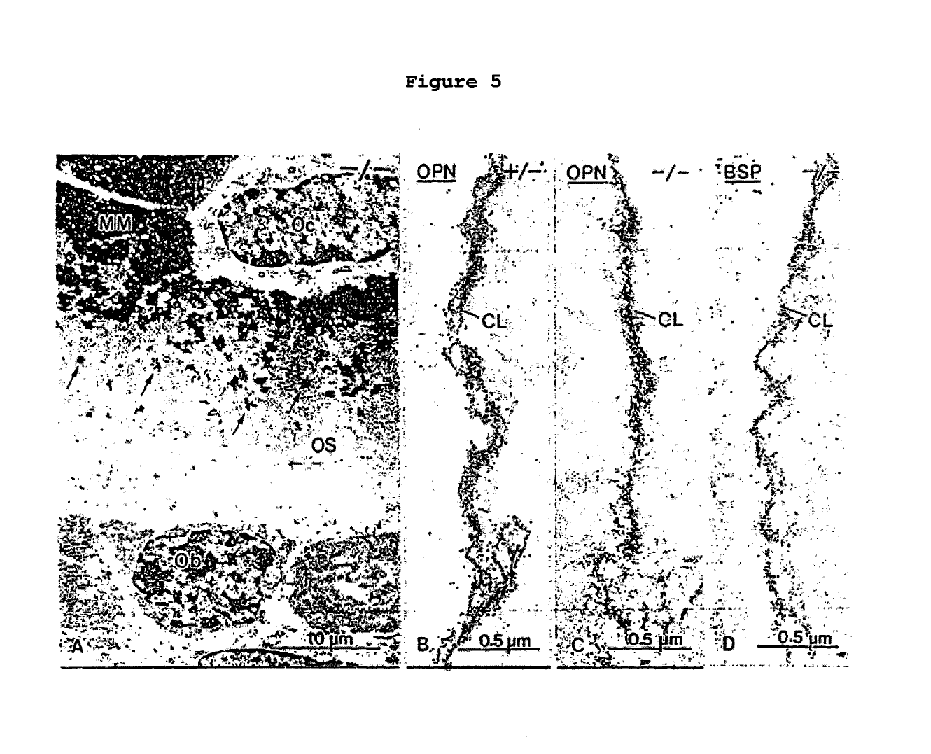

OPN has been implicated in osteoclast function^{(4,3)} so the consequences of a lack of this protein on osteoclast differentiation from monocyte precursors was assessed in vitro. When in contact with osteoblasts, and in the presence of 1α,25-dihydroxyvitamin D₃, osteoclast precursor cells from bone marrow and spleen can be induced to differentiate into osteoclast-like cells.^{(45,46)} In these coculture systems, cells derived from bone marrow and spleen differentiate in vitro over seven days into multinucleated cells with the characteristics of osteoclasts: they stain for tartrate-resistant acid phosphatase (TRAcP), resorb bone, and bind calcitonin.⁽⁴⁹⁾ Spleen cells from Opn^{-/-} animals in such cocultures gave rise to markedly more TRAcP⁺ cells than did spleen cells from Opn^{+/+} mice (Table 1, FIG. 5). Spleen cells from Opn^{+/-} animals gave an intermediate result. While the absolute number of osteoclasts formed varied among individual animals (as has been previously shown to occur⁽⁵⁰⁾), on average, about 7-fold more multinucleated cells stained for TRAcP after 7 days in culture in the Opn^{-/-} cultures as compared to the Opn^{+/+} cultures (Table 1, and data not shown). Cells derived from the Opn^{+/-} animals were on average 3-fold more efficient at forming osteoclasts than were wildtype cells (Table 1, and data not shown). These TRAcP⁺ cells derived from Opn^{-/-} spleens were confirmed as osteoclast-like in that they were able to form resorption pits in bone slices (data not shown), and the morphology of these pits was similar for both the Opn+/+ and Opn^{-/-} osteoclasts. When bone marrow cells from Opn+/+ and Opn−/− mice were placed in such cocultures with primary osteoblasts derived from wildtype mice (either 129xC57B1/6 or ddy, Table 1), a similar increase in the numbers of TRAcP⁺ cells developing in 7 days was observed, although the magnitude of the difference, 2-4 fold increased numbers of TRAcP⁺ cells in the Opn−/− cultures, was not as great as for the spleen cells.

TABLE I
FORMATION OF TARTRATE-RESISTANT ACID PHOSPHATASE-
POSITIVE MULTINUCLEAR CELLS (TRAcP + MNCs) IN
COCULTURE EXPERIMENTS WITH CALVARIAL OSTEOBLASTS

| | Total TRAcP + MNC + SD | | | |
|---|---|---|---|---|
| | osteoblast | | | |
| tissue source | genotype | +/+ | +/− | −/− |
| spleen | +/+a | 66 ± 32 | 349 ± 88 | 857 ± 90** |
| spleen | +/+a | 63 ± 23 | nd | 202 ± 103* |
| bone marrow | +/+b | 521 ± 126 | nd | 2363 ± 225** |
| bone marrow | +/+b | 936 ± 276 | nd | 2276 ± 512** |
| spleen | −/−b | 84 ± 28 | nd | 745 ± 134$ |

TRAcP positive multinuclear cells arising from spleen or bone marrow cells derived from Opn+/+ (+/+ column); Opn+/− (+/− column) or Opn−/− (−/− column) were quantitated after differentiation for seven days in the presence of osteoblasts. All stained cells with 2 or more nuclei in 4 independent wells were counted. Tissue source refers to the origin of the cell plated in co-cultures with osteoblasts.
a: osteoblasts were derived from mouse strain ddy calvaria,
b: osteoblasts derived from mouse strain 129xC57Bl/6 calvaria.
The results are expressed as ±standard deviation.
**p < 0.001;
*p < 0.05;
$p < 0.01 by Student's t-test.
nd = not determined.

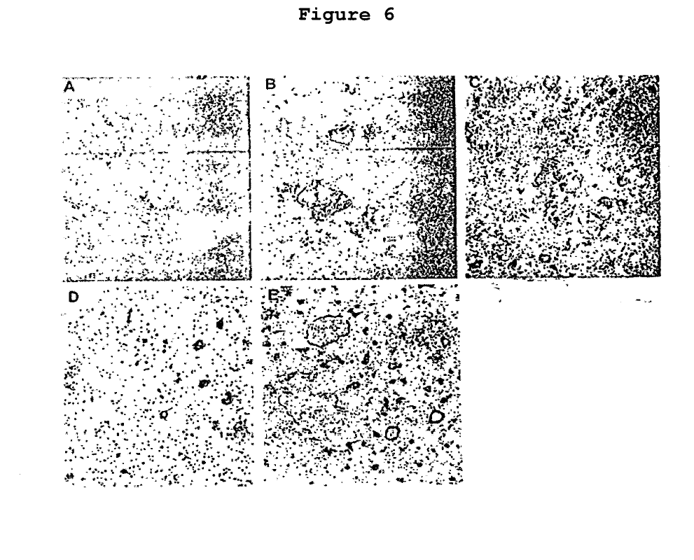

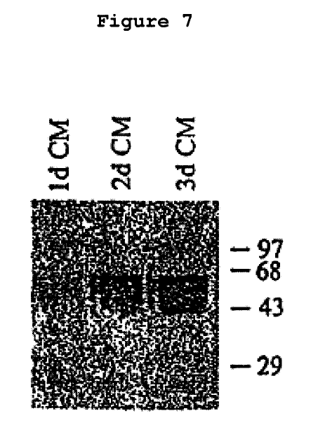

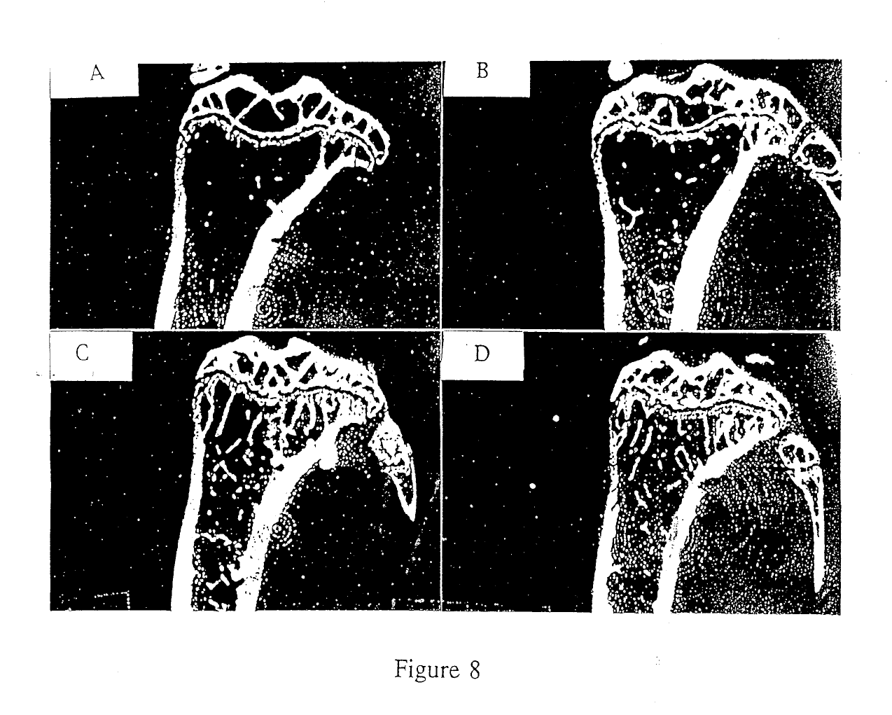

The osteoblast cells used in this coculture system produce OPN at readily detectable levels (FIG. 6), so that the osteoclasts from the Opn^{-/-} spleens were exposed to OPN during the culture period. This observation implies that the observed difference in osteoclast formation is due to differences in the spleen cells themselves, or that OPN plays an autocrine role in this system, such that the osteoclast precursors can distinguish endogenously synthesized from exogenously supplied OPN. To distinguish between these possibilities, spleen cells from Opn^{-/-} mice were differentiated on osteoblasts derived from Opn^{-/-} calvariae (Table 1). The results were similar to those obtained with wildtype osteoblasts, indicating that OPN is not required for this process in excess of the amount provided in the FBS.

### Significance of Normal Development in Opn^{-/-} Mice

The osteopontin protein sequence is highly conserved among species,⁽⁵¹⁾ and the protein is expressed by cells in a wide variety of tissues throughout the body.⁽⁵²⁾ OPN is found in most if not all body fluids, is very abundant in mineralized tissues, and has long been implicated in bone formation and remodeling.^{(3,53)} For these reasons, the apparently normal phenotype of mice lacking osteopontin was unexpected. Opn mRNA is expressed at high levels in kidney, for example, yet the kidneys of the mice which do not express OPN are morphologically normal. We have been unable to detect OPN protein in normal (+/+) kidneys by western blotting (data not shown), which implies that under non pathological conditions, there is little OPN in soft tissues. Thus, while OPN is an ubiquitous component of body fluids, perhaps acting to prevent mineral precipitation from these solutions,^{(54,55)} it does not appear to play an essential role in the normal processes of soft tissue differentiation or homeostasis. It follows that a lack of OPN in these soft tissues has little consequence to the healthy, unstressed organism. Interestingly, mice with disruptions in genes coding for vitronectin and tenascin, which are also RGD-containing proteins^{(56,57)}, or for both OPN and vitronectin⁽⁷⁸⁾ similarly develop and grow normally.

### Role of OPN in Bone Morphology and Mineralization

OPN is abundant in the mineralized tissues; its ability to bind to calcified matrices is due to its overall acidity, including a poly-Asp stretch, and a high degree of phosphorylation.⁽⁵⁸⁾ The accumulation of OPN in cement lines demarcating the reversal site of bone remodeling by osteoclasts, and at bone surfaces—laminae limitantes—where osteocytes, osteoblasts, bone lining cells and osteoclasts routinely interface directly with the extracellular matrix, has led to speculation that OPN regulates cell adhesion and dynamics at bone surfaces.^{(4,5,32)} It has also been proposed that OPN present at cement lines (resting, or reversal, lines) and elsewhere in bone mediates hard tissue integrity by binding various extracellular matrix components as well as mineral, thus linking organic and inorganic phases to provide tissue adhesion/cohesion.^{(rev: 33)}

In the present study, we have documented that morphologically defined structures known to be rich in OPN persist in the bones and teeth of Opn^{-/-} mice, and that a lack of OPN apparently has no effect on either the structure or the distribution of cells within these tissues. While no histologically detectable phenotype is apparent in the mineralized tissues of mice lacking OPN, biochemical and crystallographic studies are in progress to test for differences in bone strength and mineral organization in these animals. Since OPN is a member of a family of RGD-containing proteins, some of which, such as bone sialoprotein, are abundant in bone, it may be that some of these other proteins, or perhaps heretofore unidentified proteins, can subserve the putative function of OPN in its absence.

With regard to extracellular matrix mineralization in bones and teeth, our data suggest either that OPN is not normally involved in the calcification of these tissues or that such hard tissues can utilize alternative calcification strategies not involving OPN. A variety of anionic proteins have been identified as regulators of calcification in vertebrate and invertebrate mineralizing systems.^{(35,59,60)} In light of the vital importance of the vertebrate skeleton in maintaining form and locomotion capability, in defining internal cavities and protecting organs and tissues, and in acting as an ion reservoir for calcium homeostasis, it is reasonable that redundant strategies exist for developing and maintaining hard tissue extracellular matrices such as found in bone.

### Function of OPN in Osteoclastogenesis

Although there is no obvious alteration in the morphology or ultrastructure of bone cells and extracellular matrix in the Opn^{-/-} animals, the formation of osteoclast-like cells is enhanced up to 13-fold in cocultures with calvarial osteoblasts when the cells are prepared from the spleen or bone marrow of the Opn^{-/-} animals compared to those from the Opn^{+/+} animals. This result suggests two possibilities: first, that OPN inhibits the differentiation of osteoclast precursors into osteoclasts in cell culture, or second, that OPN affects the formation or accumulation of osteoclast precursors in the spleen and in the bone marrow. Our observation that osteoclasts are formed with similar efficiencies on wildtype and Opn −/− osteoblasts implies, however, that OPN expression is not required for this differentiation process in vitro, and that the difference observed in vitro reflects differences in the cellular composition of the spleen and bone marrow.

Yamate et al.⁽⁶¹⁾ demonstrated that in cultures of bone marrow cells a specific antiserum to OPN inhibited the formation of TRAcP⁺ cells, as did RGD-containing peptides, suggesting that the binding of OPN to cell surface integrins is important in the development of osteoclasts in the in vitro system. Our results differ from these observations in that we describe an inhibitory effect of OPN on the process of osteoclast differentiation. The major difference between our experiments and those of Yamate and coworkers is in the culture conditions: our experiments were performed on calvarial osteoblasts while those of Yamate et al. utilized cells from the bone marrow cultures themselves as stromal cells. One possible explanation for these divergent results is that there are multiple differentiation pathways leading to osteoclastogenesis, and the pathway used depends on the specific cellular and molecular composition of the culture system used. We hypothesize, then, that osteopontin plays different roles in the different pathways—stimulating differentiation along one pathway, inhibiting it along another. Indeed, our results demonstrate that OPN is dispensable for the differentiation process in vitro altogether, in that osteoclast formation occurs when Opn −/− spleen cells are cocultured with Opn −/− osteoblasts.

In any case, the alteration in osteoclast precursors that we detect in this assay does not appear to affect osteoclast differentiation in vivo under non-pathological conditions. An expected result of increased osteoclast development in vivo might be an osteoporotic/osteopenic phenotype in the Opn^{-/-} animals, yet this has not been detected. Thus, mechanisms to compensate for a lack of OPN appear to exist in the whole animal, but possibly not in the isolated cell cultures. Additionally, if different pathways of osteoclast differentiation exist in vivo, it may be that the pathway used for osteoclastogenesis during normal bone development does not depend on OPN, while a different pathway is used in pathological situations, in which OPN may have a function.

### Function of OPN in Pathological Settings

OPN expression in a variety of tissues is elevated in certain pathologies, and the protein is thought to function in several important aspects of immune cell function. For example, OPN expression is known to be increased in the kidney in association with the interstitial fibrosis occurring with glomerulonephritis, with cyclosporine nephropathy, with angiotensin II-induced tubulointerstitial nephritis, and with hydronephrosis.^{(2,26,27,62)} In each case, OPN was hypothesized to play a role in the recruitment of macrophages to these sites of tissue injury. OPN interacts with macrophages,⁽²³⁾ attenuates their response to specific stimuli,⁽¹⁷⁾ and stimulates IgG and IgM production in mixed cultures of macrophages and B cells.⁽⁶³⁾ The protein is important in macrophage infiltration in vivo⁽⁶⁴⁾, is implicated in macrophage adhesion and may also function in bone wound healing⁽⁶⁵⁾. Taken together, these observations implicate osteopontin expression as a cellular response to tissue injury of various sorts.⁽⁶⁶⁾ Indeed, Liaw et al.⁽⁷⁸⁾ have presented evidence that OPN does have a role in soft tissue remodeling, i.e. wound healing. Since the mice in our colony, housed under specific pathogen-free conditions, are not subject to such pathologies, the effect of an absence of OPN in these animals is minimal.

### Example II

### Osteopontin Knockout Mice are Resistant to Ovarectomy-induced Osteoporosis

As mentioned in the previous example, osteopontin is a ligand for the αvb3 integrin, which is expressed at high levels in osteoclasts and has been implicated in the function and development of these cells. While osteopontin-deficient mice are fertile, develop normally, and exhibit no obvious defects in their mineralized tissues, these mice are resistant to ovariectomy-induced osteopenia. Thus, osteopontin is required for the rapid bone resorption resulting from the estrogen deficiency in ovariectomized mice. Accordingly, the osteopontin-deficient mice of the invention may be used to advantage to screen therapeutic agents that are involved in the development of osteoporosis. In one such assay, therapeutic agents would be administered to ovariectomized and non-ovariectomized osteopontin deficient mice. Agents which promote osteoporosis in the ovariectomized osteopontin deficient mice would then be characterized further. In an alternative assay, therapeutic agents would be administered to non-ovariectomized and ovariectomized wild-type mice. Agents which inhibit osteoporosis in the ovariectomized, wild-type mice, would be characterized further.

Postmenopausal osteoporosis⁽⁷³⁾ is one of the most common diseases affecting aged women. It is a major health problem with regard to not only the high fracture rates and loss of quality of life of the women but also the economic loss to society. In the United States, the number of patients is estimated to be approximately 12 million and the medical costs are estimated in the billion dollar range. It is well established that withdrawal of estrogen causes loss of bone due to an increase in osteoclastic bone resorption and that supplementation with estrogen can reduce bone loss not only in humans but also in experimental animals. One the critical steps in osteoclastic bone resorption is the attachment of osteoclasts to bone and the subsequent formation of a sealing zone, which can be visualized as a clear zone by electron microscopy⁽⁷⁴⁾. This attachment is a prerequisite for bone resorption since it creates a sequestered microenvironment into which osteoclasts secrete protons, creating an acidic milieu suitable for the dissolution of bone mineral. Osteoclasts also secrete proteases into this sealed environment to digest bone proteins. Integrins are thought to function in the development of osteoclasts, osteoclastic migration to sites of resorption, and initial attachment to bone as well as formation of the sealing zone in osteoclasts⁽⁷⁵⁾. One of the characteristics of osteoclasts is the high levels of the αvβ3 integrin on the cell surface⁽⁷⁶⁾. The functional importance of integrins has been indirectly suggested by the inhibitory effect of disintegrins such as echistatin, which have been shown to block osteoclastic development, osteoclastic attachment and subsequent bone resorption in vitro. Importantly, these disintegrins block bone resorption in vivo⁽⁷⁷⁾. These observations indicate that the αvβ3 integrin plays a critical role in bone resorption. The αvβ3 integrin binds to RGDS-containing proteins such as thrombospondin, fibronectin, vitronectin, fibrinogen, von Willebrand factor and osteopontin. Among those, osteopontin has been considered to be one of the most important candidates for a natural ligand for αvβ3 integrin expressed in osteoclasts based on the in vitro experimental data. Osteopontin is one of the most abundant non-collagenous proteins in bone matrix and is produced by osteoblasts as well as osteoclasts. Osteopontin is also produced by the cells in non-skeletal tissues and has been implicated in tumorigenesis. Substrate-bound osteopontin promotes attachment of osteoclasts while soluble osteopontin can alter calcium levels in osteoclasts and suppress iNOS induction in kidney cells and macrophages. These observations suggest that osteopontin could play a key role in both cell attachment and in controlling subsequent bone cell functions such as resorption. Osteopontin has been observed to be present at high levels in the cement (renewal) lines and the lamina limitans. However, the role of osteopontin in vivo in bone metabolism has not yet been elucidated.

Bone resorption following ovariectomy is a model of post-menopausal osteoporosis. To examine the role of osteopontin in this process, we removed the ovaries of 4.5-6-month-old osteopontin-deficient mice and control mice and examined their bones 4 weeks after the operation. In control experiments, osteopontin-deficient and normal mice were sham-operated. At the four-week time point, the uterine weight of the ovariectomized wild type animals was about 30% of the sham-operated wild type mice. Similarly, the uterine weight of the ovariectomized osteopontin-deficient mice was about 25% of that of the sham operated null mice (Table II). The uterine weights of the sham-operated osteopontin-deficient mice and normal mice were similar. There was no difference in the reduction of uterine weight between osteopontin-deficient mice and wild type mice (Table II) indicating that ovariectomy affects organs such as uterus similarly in both osteopontin-deficient and wild type mice. Likewise, the body weight of the sham-operated or ovariectomized animals was similar in both osteopontin null and wild type mice.

TABLE II

| | MEAN +/− SD | n |
|---|---|---|
| WT OVX | 0.026* +/− 0.006 | 4 |
| WT SHAM | 0.081 +/− 0.021 | 4 |
| KO OVX | 0.024 +/− 0.008 | 4 |
| KO SHAM | 0.105 +/− 0.039 | 4 |

*uterine weight (gram)

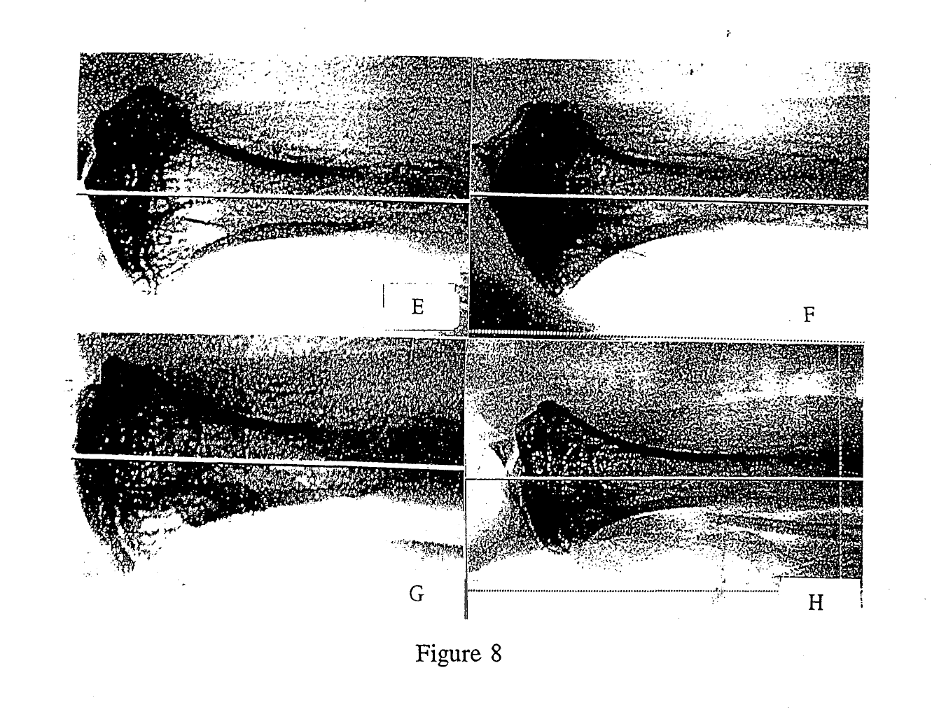

Bone volume was measured quantitatively using micro-computed tomography (μCT) of the proximal epiphyses of the tibiae. The morphology was evaluated in the mid-sagittal planes as shown in FIGS. 8 A,B,C and D. In two dimensional images, the trabecular bones were seen to be longer and more connected in the sham operated osteopontin-deficient mice (FIG. 8D) compared to the trabecular bones in sham-operated wild type mice (FIG. 8B). The trabecular bones of the ovariectomized wild type mice were sparse (FIG. 8A) compared to, sham operated wild type (FIG. 8B). However, the most striking, feature in the osteopontin-deficient mice was the similar morphology of the trabecular bones between the ovariectomized and sham-operated animals (FIGS. 8C, D). The cutting plane of section is indicated in the FIGS. 8 E-H.

Quantitation of the two dimensional (2D-) bone volume in the tibiae shown in FIG. 8A to 8D using automated image analyzer indicated that the bone volume, expressed as bone area; per tissue area of the wild type mice was reduced by 40% at four weeks following ovariectomy (9.8%) as compared to sham-operated wild type animals (16.1%) while no reduction was observed in the osteopontin-deficient mice between ovariectomy (23.2%) and sham-operation (23.0%) (Table 3). Furthermore, the quantification revealed more bone volume in sham-operated osteopontin-deficient mice (23.0%) than sham-operated wild type mice (16.1%).

TABLE III

| | MEAN | SD | n |
|---|---|---|---|
| WT OVX | 9.80* | 1.10 | 4 |
| WT SHAM | 16.10 | 1.46 | 4 |
| KO OVX | 23.20 | 4.19 | 5 |
| KO SHAM | 23.00 | 5.60 | 5 |

*bone volume (%)

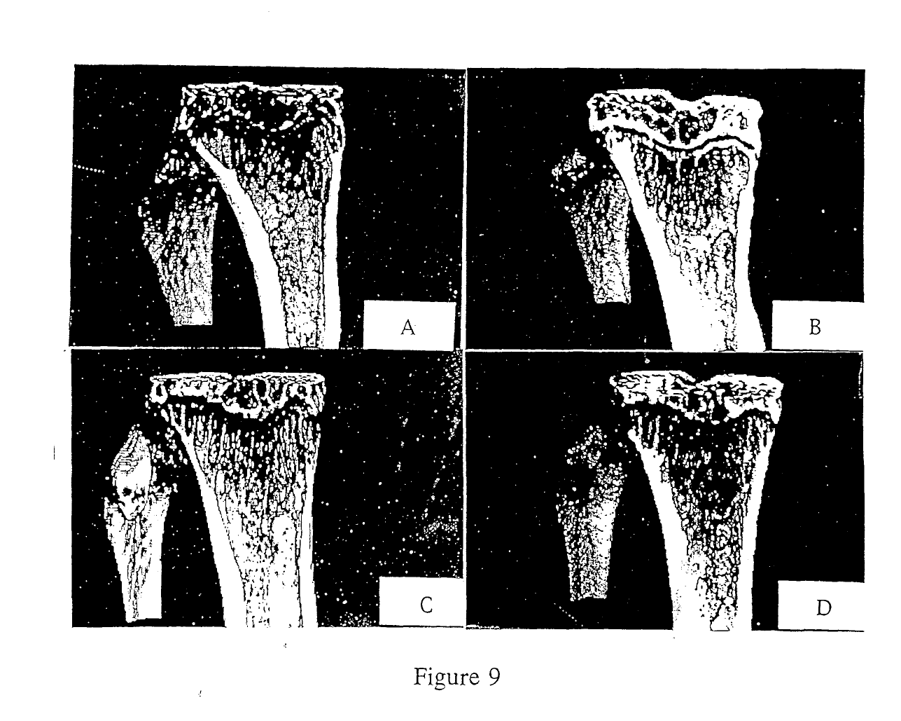

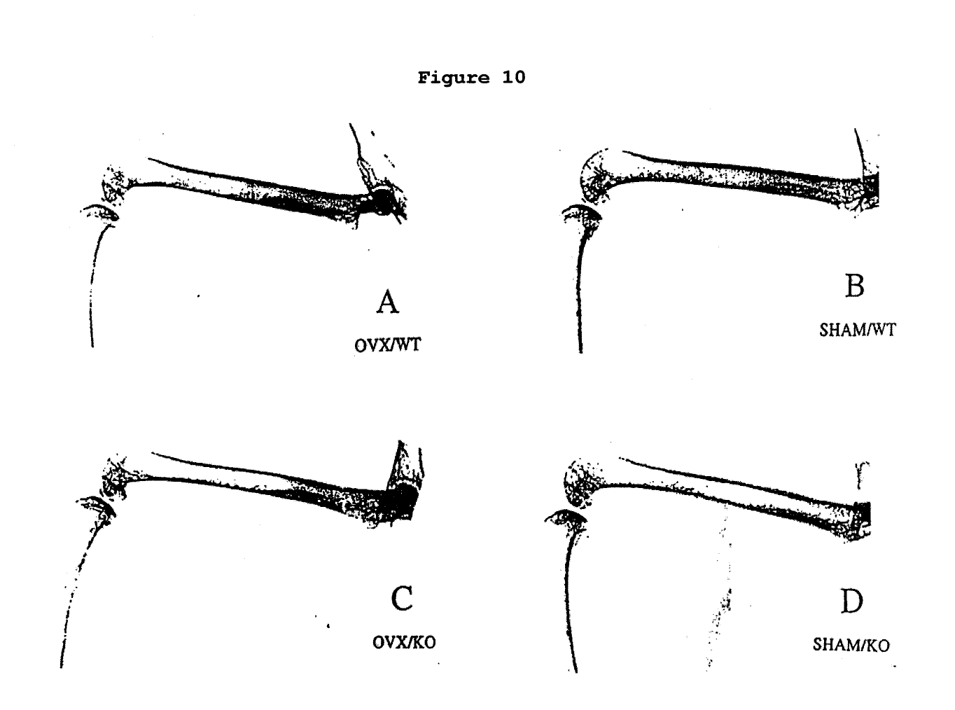

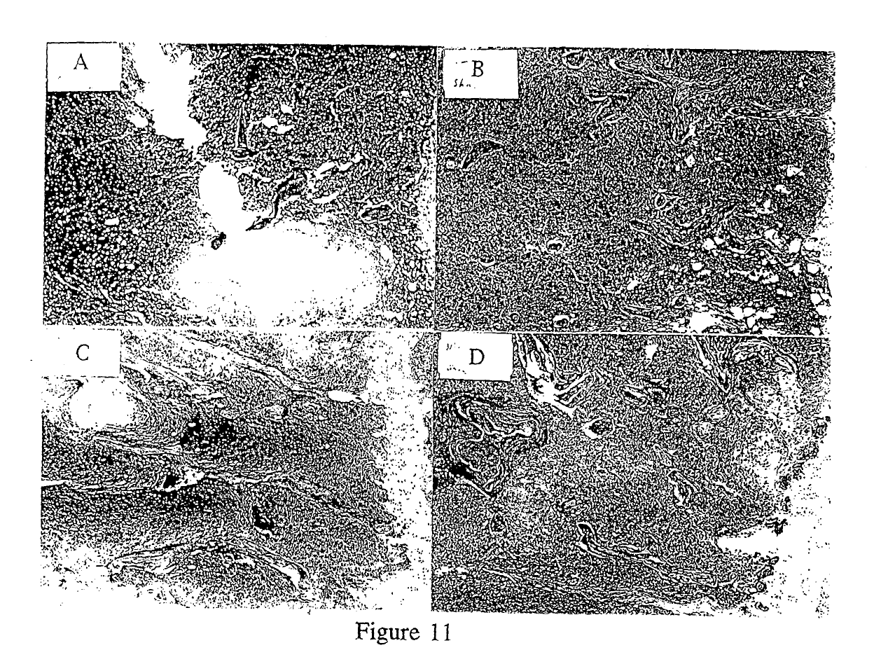

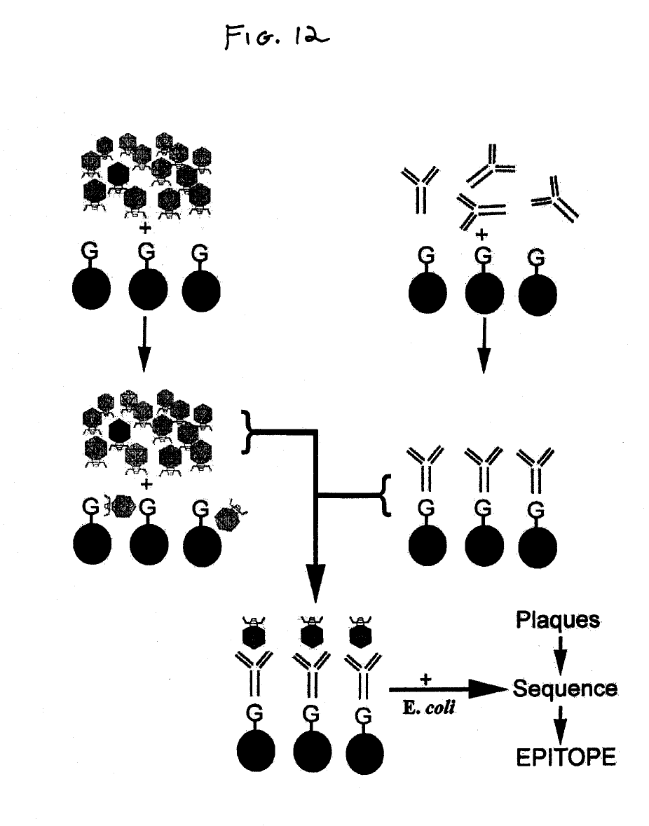

Three dimensional(3D-) structures of the trabecular bones indicate the reduction in length, number and connectivity of the trabeculae in ovariectomized wild type animals compared to sham-operated wild type while no decrease of these were observed in ovariectomized osteopontin-deficient mice compared to sham-operated osteopontin-deficient mice, supporting the findings observed in two dimensional analyses (FIG. 9). Soft X-ray examination also revealed the preservation of the longer trabecular bones in the epiphyseal and metaphyseal regions of the osteopontin-deficient mice compared to the wild type and ovariectomy reduced the trabecular bones in wild type mice but not in osteopontin-deficient mice (FIG. 10). The reduction is observed mainly in epiphyseal and metaphyseal bone area, although it is also observed in the ends of the mid shaft area in ovariectomized wild type. On the other hand, osteopontin-deficient mice show small trabeculation which extends into the diaphyseal region. This extended trabeculation starting from the metaphysis and continuing into the ends of the diaphyses was not reduced even after ovariectomy in osteopontin-deficient mice (FIG. 10). Histological sections also revealed that more bone volume in ovariectomized osteopontin-deficient mice compared to the ovariectomized control (FIG. 11). Bone islands are apparently more and longer in osteopontin-deficient mice in both sham and ovariectomized animals. Cellularity in the bone marrow was similar between the wild type and osteopontin-deficient mice regardless of the ovariectomy or sham operation.

As reported previously, bone marrow and spleen cells prepared from these osteopontin-deficient mice differentiated into osteoclasts in vitro in the presence of osteoblasts and vitamin D. We also showed that the number of osteoclasts generated in vitro in cocultures of cells prepared from osteopontin-deficient bone marrow and spleen with the osteoblasts from the calvariae of osteopontin-deficient mice was significantly greater than the number of osteoclasts developed in the cocultures of the cells prepared from the wild type mice. See Example I. These results indicate that there is no defect in osteoclastogenesis in the osteopontin-deficient estrogen-sufficient mice. Furthermore, these in vitro generated osteoclasts could resorb bone slices prepared from normal bovine femora. It seems that osteopontin-deficient mice are resistant to the ovariectomy-induced bone resorption not because of the lack of osteopontin produced by the osteoclasts which are resorbing bones but rather because of altered osteoclast regulation in the absence of osteopontin.

The 2D-pattern, radiographical density and 3D-μCT morphology of the remaining trabecular bones of the ovariectomized osteopontin-deficient mice were similar to those of the sham operated osteopontin-deficient mice. The data suggest that the main defect induced by osteopontin-deficiency is a reduction in the ovariectomy-induced osteoclastic bone resorption activity. Our observations on the resistance against ovariectomy-induced bone resorption in osteopontin-deficient mice by itself clearly indicate the importance of osteopontin in this estrogen-deficiency-induced osteoporosis model. The next step, currently in progress is to understand how the loss of osteopontin leads to such resistance to bone resorption in vivo.

In summary, we have demonstrated that osteopontin-deficient mice are resistant to bone resorption induced by ovariectomy. A similar resistance to ovariectomy induced osteopenia was also observed in opn−/− mice generated in a pure 129 Sv background (data not shown). Whether this is true for human post-menopausal osteoporosis will require further investigation in humans. As of now, there is no information regarding osteopontin deficiency in humans, who might be expected to show resistance to post-menopausal osteoporosis. Genetic analysis of osteopontin gene polymorphism may predict certain patients who could have high or low risk of post menopausal bone loss. If osteopontin does play a role in human post menopausal osteoporosis, it could provide further support for the endeavor to develop anti-bone-resorptive drugs, particularly measures to suppress the action of OPN.

### Example III

### Use of Osteopontin Knockout Mice for the Generation of Osteopontin Specific Monoclonal Antibodies

As mentioned in the previous examples, osteopontin is a widely expressed protein that has been conserved throughout evolution. The extensive similarity of osteopontin proteins among species presents certain problems for the generation of osteopontin-specific monoclonal antibodies. Antibodies are generated in response to exposure to foreign antigens. The foreign antigens must be recognized as “non-self” before an immune response will be mounted. The osteopontin knock out mice of the invention can be used to advantage for the production of osteopontin antibodies as these animals do not express native osteopontin. Antibodies so generated will provide a useful research tool for intracellular localizations, epitope mapping and immunoprecipitation studies for characterizing those proteins that form intracellular associations with osteopontin. This concept may also be expanded to encompass antibodies specific for any highly conserved plasma protein. Knock out mice having a null mutation for the gene encoding the plasma protein of interest may be utilized for the generation of a wide array of monoclonal antibodies immunospecific for those proteins. Utilization of knock out mice for this purpose ensures that the immunizing protein antigen will be recognized as non-self and therefore invoke a powerful immune response.

Additional potential applications for the antibodies of the invention include assays to determine whether a particular epitope on the osteopontin protein has been modified. Such antibodies may be used to advantage to assess post-translational modifications or modifications associated with a particular disease state, such as particular cancers, certain kidney or vascular pathologies or immune system disfunctions. The antibodies of the invention may also be utilized to inhibit osteopontin action. For example, loss of bone during osteoporosis appears to require the presence of osteopontin in the bone. A monoclonal antibody immunologically specific for a determinant critical for this interaction may prevent osteopontin from stimulating the bone resorption that occurs during osteoporosis. As mentioned previously, osteopontin inhibits nitric oxide production. In certain inflammatory conditions where nitric oxide production is required or beneficial, a monoclonal antibody specific for osteopontin might prevent osteopontin from inhibiting this beneficial nitric oxide production. Finally, the monoclonal antibodies of the invention may be used to quantify various species of osteopontin, for example in ELISA reactions. It is likely that osteopontin levels in plasma deviate from normal with particular disease states. Thus, the ability to easily and accurately quantify osteopontin levels would be clinically useful.

Polyclonal antibodies can be raised by administration of osteopontin to the knockout mice of the invention, using known immunization procedures. Usually a buffered solution of the antigen accompanied by Freund's adjuvant is injected subcutaneously at multiple sites. A number of such administrations at intervals of days or weeks is usually necessary. A number of animals, for example from 3 to 20, is so treated with the expectation that only a small proportion will produce good antibodies. The antibodies are recovered from the animals after some weeks or months.

The use of monoclonal antibodies is particularly preferred because they can be produced in large quantities and the product is homogeneous. The preparation of hybridoma cell lines for monoclonal antibody production derived by fusing an “immortal” cell line and lymphocytes sensitized against the immunogenic preparation can be done by techniques which are well known to those who are skilled in the art. See, for example, Doullard, J. Y. and Hoffman, T., “Basic Facts About Hybridomas” in Compendium of Immunology, vol. II, L. Schwartz (ed.) (1981); Kohler, G. and Milstein, C., Nature, 256:495-497 (1975); Koprowski, et al., European Journal of Immunology, 6:511-519; Koprowski et al., U.S. Pat. No. 4,172,124; Koprowski et al., U.S. Pat. No. 4,196,265; and Wands, U.S. Pat. No. 4,271,145; the teachings of which are herein incorporated by reference.

Unlike preparation of polyclonal sera, the choice of animal for monoclonal antibody production is dependent on the availability of appropriate “immortal” lines capable of fusing with lymphocytes thereof. Mouse and rat have been the animal of choice in hybridoma technology and preferably used. Humans can also be utilized as sources of sensitized lymphocytes if appropriate “immortalized” cell lines are available. For the purpose of the present invention, the osteopontin knockout mice may be injected with approximately 0.1 mg to about 20 mg of purified osteopontin or fragments thereof. Usually the injecting material is emulsified in Freund's complete adjuvant. Boosting injections may also be required. The detection of antibody production can be carried out by testing the antisera with appropriately labeled antigen, as required by radioimmunoprecipitation, or with capture complex, as required by a variety of solid phase immunoassays including competitive ELISA. Lymphocytes can be obtained by removing the spleen or lymph nodes of sensitized animals in a sterile fashion and carrying out cell fusion. Alternatively, lymphocytes can be stimulated or immunized in vitro, as described, for example, in C. Reading, J. Immunol. Meth., 53:261-291, (1982).

A number of cell lines suitable for fusion have been developed, and the choice of any particular line for hybridization protocols is directed by any one of a number of criteria such as speed, uniformity of growth characteristics, absence of immunoglobulin production and secretion by the nonfused cell line, deficiency of metabolism for a component of the growth medium, and potential for good fusion frequency.

Intraspecies hybrids, particularly between like strains, work better than interspecies fusions. Several cell lines are available, including mutants selected for the loss of ability to secrete myeloma immunoglobulin. Included among these are the following mouse myeloma lines: MPC sub II-X45-6TG, P3-NS1-1-Ag4-1. P3-X63-Ag8, or mutants thereof such as X63-Ag8.653, SP2-O-Ag14 (all BALB/c derived), Y3-Ag1.2.3 (rat) and U266 (human).

Cell fusion can be induced either by virus, such as Epstein-Barr or Sendai virus, or by polyethylene glycol. Polyethylene glycol (PEG) is the most efficacious agent for the fusion of mammalian somatic cells. PEG itself may be toxic for cells, and various concentrations should be tested for effects on viability before attempting fusion. The molecular weight range of PEG may be varied from 1000 to 6000 da. The ratio between lymphocytes and malignant cells is optimized to reduce cell fusion among spleen cells and a range of from about 1:1 to about 1:10 (malignant cells:lymphocytes) gives good results.

The successfully fused cells can be separated from the myeloma line by any technique known in the art. The most common and preferred method is to choose a malignant line which is Hypoxanthine-Guanine Phosphoribosyltransferase (HGPRT) deficient, which will not grow in an aminopterin-containing medium used to allow only growth of hybrids and which is generally composed of hypoxanthine 1×10⁻⁴ M, aminopterin 4×10⁻⁷ M and thymidine 1.6×10⁻⁵ M, commonly known as HAT medium. The fusion mixture can be grown in the HAT-containing culture medium immediately after the fusion. Cell culture usually entails maintenance in HAT medium for one week and then feeding with either regular culture medium or hypoxanthine, thymidine-containing medium.

The growing colonies are then tested for the presence of antibodies that recognize osteopontin. Detection of hybridoma antibodies can be performed using an assay where the capture complex is bound to a solid support and allowed to react with hybridoma supernatants containing putative antibodies. The presence of antibodies may be detected by direct ELISA techniques using a variety of indicators. Most of the common methods are sufficiently sensitive for use in the range of antibody concentrations secreted during hybrid growth.

### Human OPN Gene Fragment Library

A human OPN gene fragment library was constructed employing the Novagen T7SelectPhage Display system according to the manufacturer's instructions (Novagen). Briefly, an OPN plasmid which encodes the full length human OPN molecule (OPNlb/Harpo4) [Young et al., 1990 Genomics 7:491-502; Rollo, 1995] was digested with DNase I. DNA fragments between approximately 50-150 by were ligated into the T7Select415-lb vector using EcoRI adapters. In vitro packaging reactions were performed as described in the Novagen T7Select System Manual. PCR was performed using the primers T7SelectUP (5′GGAGCTGT. CGTATTCCAGTC-3′) and T7Select Down (5′-AACCCCTCAAGACCCGTTTA-3′) which flank the T7Select-lb multicloning site. Positive clones were defined as those with PCR products >30 bp larger than products from an empty vector. The concentration of unique phage in the T7 human OPN library was determined to be 8×10⁵

### Biopanning and Epitope Determination

For epitope determination, the Novagen T7 Select protocol was followed, and a modified biopanning protocol was developed (see FIG. 12). Approximately 10¹² phage were pre-incubated at room temperature in hypoxanthine-thymidine medium with 10% Protein G-agarose beads (Pierce Biotech) to remove non-specific binding phage. Antibody-containing supernatants were simultaneously mixed at room temperature for 30 min with a concentration of 10% (v/v) Protein G beads. Both mixtures were then centrifuged at −3000 g. The phage-containing supernatant was added to the precipitated antibody-protein G pellet and incubated with mixing for 1 hr. After incubation and multiple PBS washes, the mixture was centrifuged as previously and the pellet added directly to log phase BL21 E. coli. The bacteria were immediately added to 3 ml of molten top-agarose and plated. Positive plaques were identified by incubating plaque lifts with the desired antibody. Positive plaques were dispersed in 10 mM EDTA, pH 8.0 and heated for ten min at 65° C. to disrupt the phage. The mixture was then clarified by centrifugation at 14,000×g and used for PCR amplification of the insertion region with the T7Select Up and Down primers. PCR products were sequenced and osteopontin amino acid sequences were determined and aligned.

### Cell Culture

MC3T3E1 subclone 4 cells (kind gift from Dr. R. Franceschi, University of Michigan) were maintained in α-MEM (Invitrogen Corp., Carlsbad, Calif.) with 10% FBS (Hyclone, Logan, Utah), 5 μg/ml penicillin, 5 U/ml streptomycin and 2 mM glutamine. For differentiation, cells were grown until confluent then switched to growth medium above containing 100 μg/ml ascorbic acid and 10 mM β-glycerophosphate (Sigma-Aldrich, St. Louis, Mo.) for an additional 10-12 days before generating conditioned medium. Ras-transformed fibroblasts (275-3-2), or the parental non-transformed cells 3T3-275 [Wu et al., 20001, were maintained in DMEM (Mediatech Inc., Herndon, Va.) with 10% FBS (Hyclone, Logan, Utah), 5 μg/ml penicillin, 5 U/ml streptomycin and 2 mM glutamine. Conditioned medium was generated from these confluent cell cultures by overnight incubation with serum-free medium.

### Western Blotting

Freshly collected conditioned medium was used for western blotting of OPN produced by cell lines. Typically 10-20 μl/lane of conditioned medium was fractionated by SDS polyacrylamide gel electrophoresis (PAGE) with 12% gels. For purified proteins, equal amounts (typically 50 ng) in each lane were used. Protein was transferred to PVDF membranes (Millipore, Billerica, Mass.), which were cut into strips and blotted with 1 μg/ml of purified monoclonal antibodies. Antibodies were purified from hybridoma-conditioned medium using protein G-agarose beads following the manufacturer's instructions (Pierce Biotech). Human urine was collected and dialyzed extensively against 0.1M NaCl, then concentrated approximately 10-fold with Centriprep spin columns (Millipore). The equivalent of 50 μl of urine/lane was assayed by SDS-PAGE as above.

### Peptide Affinity Assay

Biotin-tagged osteopontin peptides (kind gift from Dr. Lawrence Steinman, Stanford Univ.) were added to Neutra-Avidin coated 96-well plates (Pierce Biotech) at 10 μg/ml. Anti-OPN monoclonal antibodies were added at 5 μg/ml and detected with a Alexafluor 594-conjugated anti-mouse IgG (Invitrogen) at 2 μg/ml. Fluorescence was detected using excitation/emission wavelengths of 5841612 nm with a Fluoroskan-Ascent fluorometer (Thermo Fisher Scientific Inc., Waltam, Mass.).

### Cell Adhesion Assay

Flat-bottom 96-well tissue culture-treated polystyrene microtiter plates (Corning, N.Y.) were coated with 100 μl recombinant his-tagged human OPN [Rollo, 19951 (5 μg/ml) or fibronectin (2.5 μg/ml) in phosphate-buffered saline at 4° C. overnight and blocked with 1% BSA. MDA-MB-435 or 275-3-2 cells were trypsinized, washed and resuspended in Dulbecco's modified Eagle's Medium containing 1 mg/ml BSA. Cells (5×10⁴) were added to coated wells and allowed to adhere for 3 or 3.5 hr. Non-adhered cells were removed as described by Goodwin and Pauli [I995] with slight modifications. Cells were washed twice by pipetting 75 μl Percoll wash solution (73% Percoll (Sigma), 0.9% NaCl) slowly down the sides of the wells and adherent cells were fixed by adding 50 μl fixative (10% glutaraldehyde in Percoll) in the same manner. The wash and fixative solutions were then washed from the wells with 2-3 washes of 100 μl PBS. Fixed cells were stained with 100 μl 0.1% crystal violet (25 min), washed with tap water and solubilized in 50 μl 0.5% Triton X-100 at least 1 hr before reading at 570 nm in a MRX Revelation Reader (Thermo Labsystems).

### Results

### Creation of Monoclonal Antibody Producing Hybridomas

OPN-deficient mice created in our laboratory mounted a strong immune response after immunization with recombinant OPN. Monoclonal antibody-producing hybridoma cell lines created from multiple fusions were screened for their ability to bind OPN via ELISA. Over 1000 clones were screened and 20+ lines were positive in our initial ELISA screen. Seven of these anti-OPN monoclonal antibodies will be detailed here and were selected based upon their performance in various immunoassays and their binding locations.

### Epitope Determination

We used a gene fragment display strategy to determine the epitopes of OPN bound by the antibodies which were positive in the initial screening [Kowalski, 2005]. We chose to employ the Novagen T7Select4 15 vector due to the robust nature of the T7 phage and high copy number (415) of the gene fragment on the phage surface. A human OPN expression plasmid was DNAsel digested and fragments of 50-150 bps were cloned into the T7Select415 vector. A library of approximately 8×10⁵ clones was created and amplified.

Initially, the standard manufacturer's biopanning protocol (Novagen) in which the antibodies were coated onto microtiter plates and then subjected to library panning, elution, and amplification of bound phage was employed. This method was successful for three of the monoclonal antibodies chosen for further study. Since the highest affinity binders are the most difficult to elute, and to minimize background non-specific phage binding, we modified the biopanning protocol. In the modified protocol, selection for phage displaying antibody epitopes is performed in solution utilizing antibody-protein G agarose beads to isolate bound phage.

These precipitated complexes can then be mixed directly with the host bacteria, and plated to form plaques (FIG. 12), thus avoiding the elution step. We observed over a 100-fold increase in positive binding phage and were able to epitope map antibodies that resulted in no positive clones utilizing the immobilized antibody methodology.

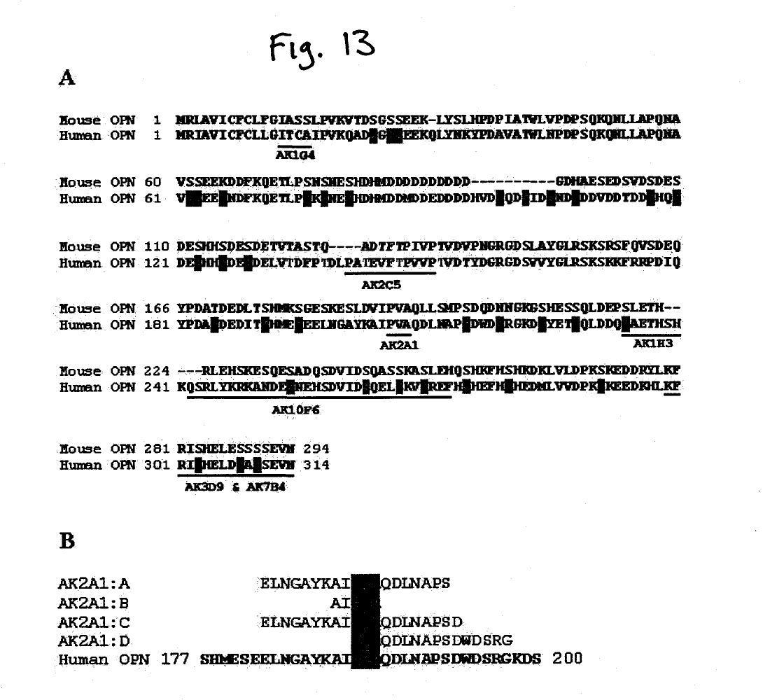

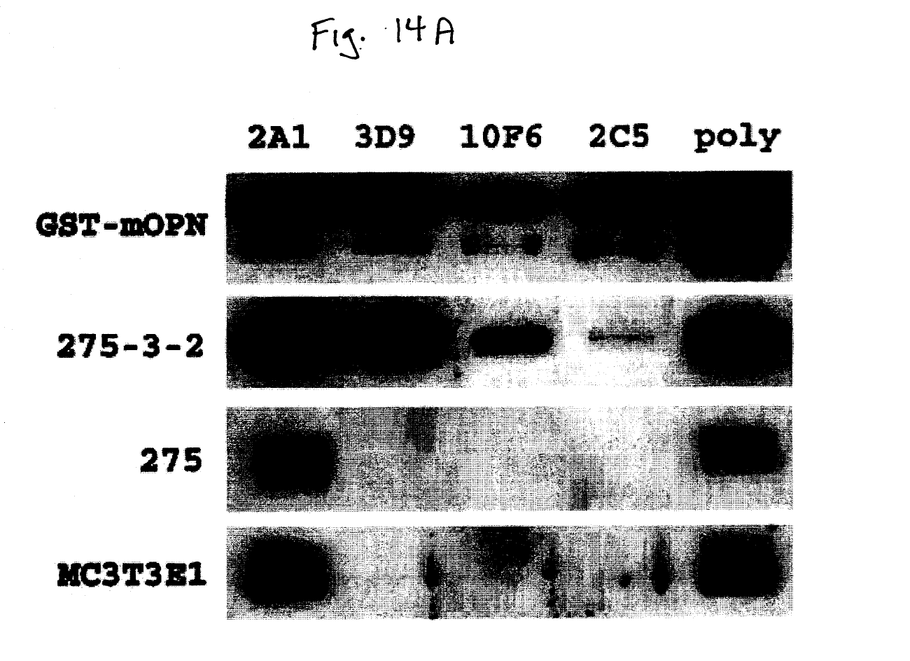

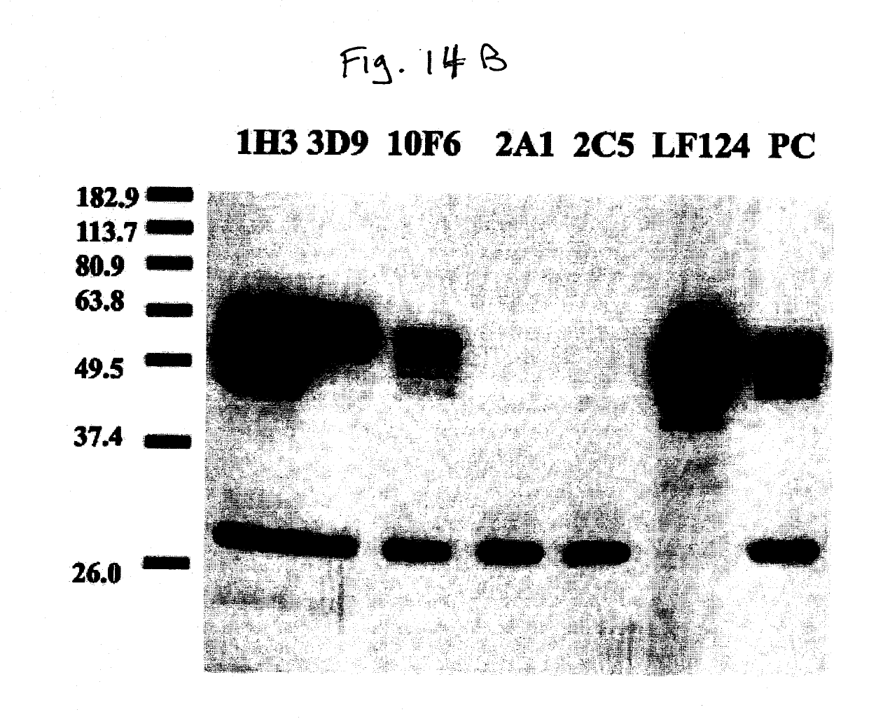

We have mapped the epitopes of seven anti-OPN monoclonal antibodies. Interestingly, five of the seven antibodies mapped to the carboxy terminal half of the OPN molecule, and one antibody (AK1G4) mapped to the signal sequence (FIG. 13A). The antibodies AK1H3 and AKIG4 only recognize human OPN, while the other antibodies are able to bind both human and murine OPN. FIG. 13B shows the results of the phage screening assay for antibody AK2A1. The screening yielded multiple peptides that were aligned to determine the minimal epitope in this case, PVA. Two of the antibodies, AK3D9 and AK7B4 recognized the same region in the extreme carboxy terminus of the molecule. The antibody AK10F6 did not yield multiple overlapping peptides from the T7 phage library screening, but yielded the same peptide multiple times.

### Antibody Binding to Osteopontin

As the OPN-knockout mice were originally immunized with recombinant OPN, we hypothesized that the presence of post-translational modifications on native OPN may prevent binding of some of the antibodies. The ras-transformed fibroblast cell line (275-3-2) produces abundant amounts of OPN (FIG. 14A), and this protein was recognized by all the antibodies tested (AK1G4, which recognizes the signal sequence, and AK1H3 which does not bind murine OPN, were not tested). On the other hand, OPN from medium conditioned by differentiating MC3T3E1 pre-osteoblasts was recognized by only one of the antibodies (AK2A1). The antibodies AK2C5, AK3D9, and AK10F6 show little to no signal, even after longer exposure. The 275-3-2 cells were derived from the parental line 3T3-275 by transformation with ras^{val12} [Wu et al, 20001. Interestingly, OPN from these non-transformed cells was similar to that from the osteoblast cells, and was only recognized by antibody AK2A1. We have recently shown that there are approximately 17 additional phosphate modifications on the MC3T3E1-produced OPN compared to the 275-3-2 ras-transformed fibroblast OPN [Christensen et al., 2007], which may explain the observed differences in antibody recognition.

FIG. 14B shows western blot results using the monoclonal antibodies to detect OPN present in human urine. Four closely migrating species of OPN are observed [Kleinman et al., 2004, A. Beshensky and J. Wesson personal communication] and the antibody showing the most intense binding to the four species was AK1H3. AK10F6 and two different polyclonal antibodies were able to recognize all 4 forms of OPN. AK3D9 binds the C-terminal region of OPN, and strongly recognized only the two higher molecular weight forms of OPN. Curiously, AK2A1 and AK2C5 showed no binding to urine OPN. This suggests that urine OPN is glycosylated (blocking AK2C5 binding), and may contain additional, possibly unique, modifications which are able to specifically inhibit AK2A1 binding. The approximately 30 kDa protein is a nonspecific species cross-reacting with the goat anti-mouse secondary antibody used. From these results we hypothesized that the majority of the antibodies generated recognize sites subject to post-translational modifications of OPN.

### Antibody Binding Sensitivity to PTMs

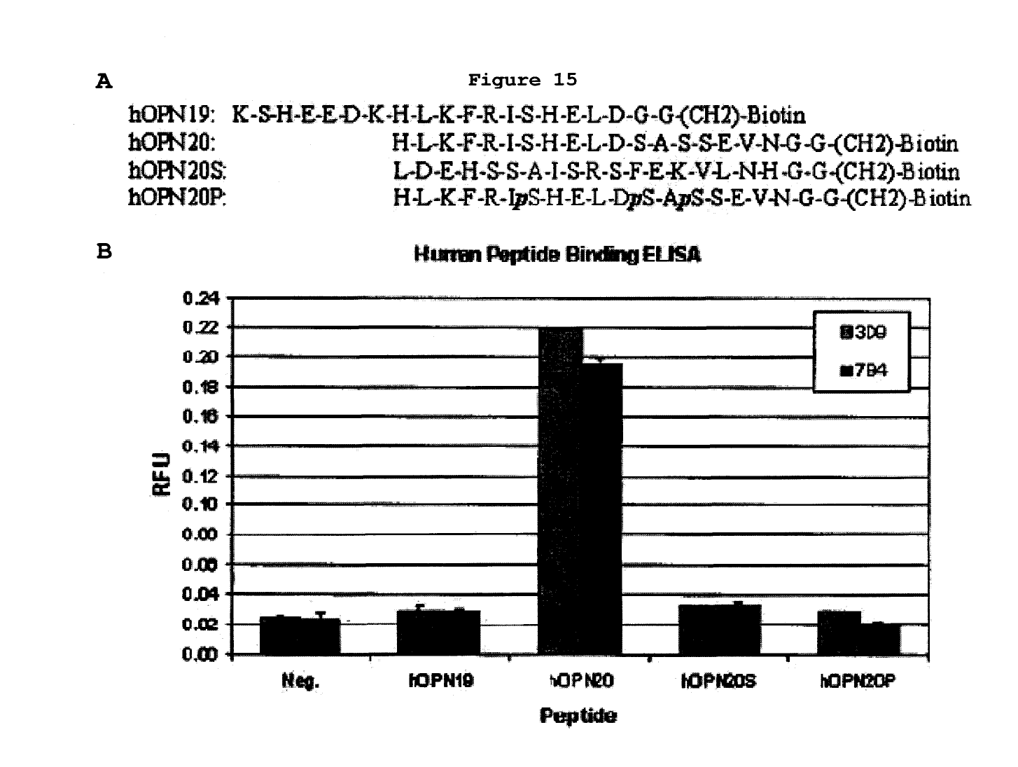

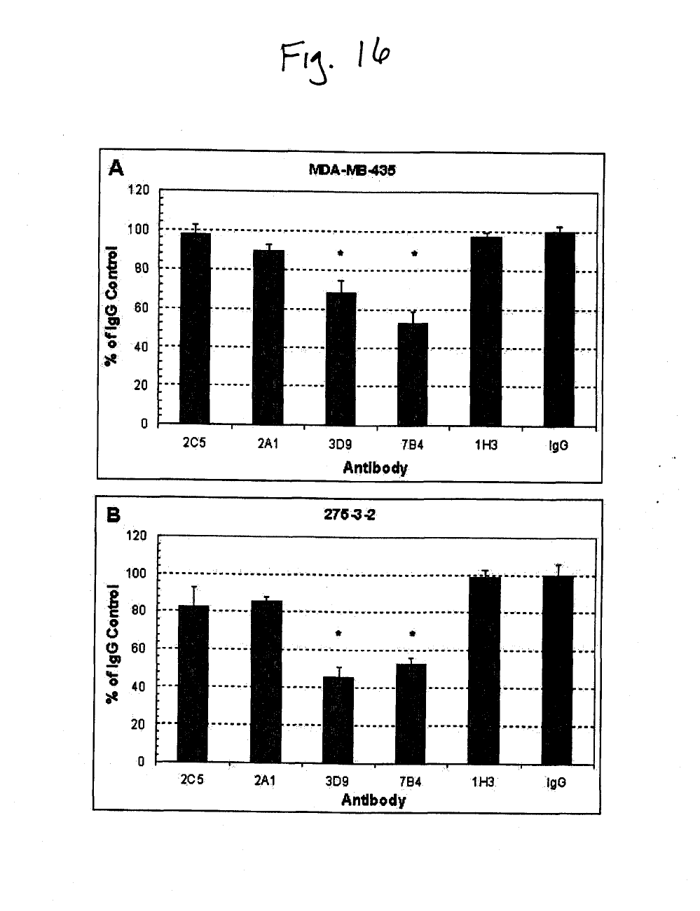

In order to test this hypothesis, a peptide binding assay was employed. Synthetic peptides corresponding to the carboxy-terminus of the human OPN molecule (FIG. 15A), including both phosphorylated and non-phosphorylated forms, and a scrambled amino acid control peptide, were used in a modified ELISA system. After the biotinylated peptides were bound to Neutraavidin plates, AK3D9 and AK7B4 antibody binding was determined. As shown in FIG. 15B, the binding to the non-phosphorylated peptide was very strong, whereas binding to both the scrambled sequence peptide and the phosphorylated peptide was similar to background. Similar results were obtained using phosphorylated and non-phosphorylated murine peptides (data not shown).

FIG. 13A also shows the sequence of human milk osteopontin with post-translational modifications noted as determined by Christensen et al. [2005]. The AK2A1 antibody is the only one whose epitope does not contain sites of post-translational modification in the mature protein. The AK2C5 antibody binds an area that is O-glycosylated, explaining why this antibody exhibits decreased binding affinity to all forms of native OPN assayed. The remaining antibodies have been determined to bind to regions of the OPN molecule containing phosphorylations that may interfere with epitope recognition by these antibodies.

### Inhibition of Cell Adhesion

Since many of the antibodies recognize the C-terminal half of the OPN molecule, where the CD44 receptor has been shown to mediate adhesion, the ability of our antibodies to inhibit the adhesion of cells to wells coated with recombinant OPN was then assessed. Antibodies were added to wells pre-coated with human recombinant his-tagged OPN (hisOPN) and allowed to bind OPN. The wells were then washed prior to adding human MDA-MB-435 breast cancer cells or 275-3-2 murine ras-transformed fibroblasts. The ability of the antibodies to block adhesion of these cells to hisOPN was assessed by comparing the adhesion to that in wells blocked with nonspecific mouse IgG (FIG. 16). The results show that for both cell lines examined, the AK3D9 and AK7B4 antibodies are able to inhibit cell adhesion by approximately 40-50%, possibly by interfering with the CD44 receptor. Similar results were obtained with plates coated with native OPN purified from medium conditioned by ras-transformed fibroblasts (data not shown), which is weakly phosphorylated, containing an average of 4 phosphates per molecule [Christensen et al., 2007].

### Discussion

Phage display is a powerful technique that allows for rapid protein-protein interaction and provides a direct link between the phage phenotype and genotype [Dunn, 1996; Smothers et al., 2002]. Biopanning has the potential to enrich for a phage of interest that is rare in an initial library. However, high stringency conditions are required to enrich for phage with the highest affinities and to avoid low affinity phage and background [Smothers et al., 2002]. In many cases these elution conditions fail to release the most strongly bound phage. Some phage display vectors have incorporated protease cleavage sites to overcome this problem (Jestin et al., 2001).

This work describes a novel modification of the standard protocol for mapping linear epitopes of monoclonal antibodies using phage display. While others have combined protein G precipitation with phage display, this was not for the purpose of epitope mapping, or to isolate antibody-bound phage (Cui et al., 2003). The modification described here has several benefits compared to the standard microplate biopanning protocol. First, the use of protein G to precipitate antibodies allows for the use of complex mixtures such as ascites or hybridoma supernatant directly, without additional antibody purification. Second, phage can be pre-cleared to lower non-specific binding, and antibody-phage complexes are allowed to form in solution, decreasing background. Third, since the bound phage remain infective, the protein G-antibody phage complexes can be directly added to bacterial cultures and plated for plaque formation, thereby eliminating the troublesome elution step. Finally, this method is much faster than the standard protocol. This modified technique allowed for the identification of the epitopes of anti-OPN antibodies which were not identified using the standard microplate biopanning protocol, suggesting that this modification lends increased sensitivity to the protocol.

The antibodies described herein are useful in determining the phosphorylation or glycosylation state of various regions of the OPN molecule. This has been demonstrated in the western blots of conditioned medium from multiple cell lines shown in FIG. 14. All of the antibodies in our panel are able to bind to OPN in medium conditioned by ras-transformed fibroblasts (275-3-2), however only AK2AI is able to recognize OPN in medium conditioned by a non-transformed fibroblast line or from differentiated MC3T3E1 osteoblasts. These data suggest that the OPN produced by the overexpressing ras-transformed cells, is less posttranslationally modified than protein made by non-ras transformed cells. OPN from a similar set of non-transformed and ras-transformed mouse NIH3T3 fibroblasts [Chambers et al., 1993] reacted similarly with the panel of antibodies as the 275 and 275-3-2 cells. Osteopontin has been shown to be upregulated in a variety of cancers, and our data suggest that the OPN produced by certain tumors may have significantly fewer PTMs than that made by normal cells.

The antibodies described were also able to distinguish differences in OPN species found in human urine. For instance, the C-terminal antibody AK3D9 did not recognize the lower two of the four bands, suggesting that these bands are either more phosphorylated than the protein in the upper bands, or represent C-terminal truncated fragments of OPN. The ability of these antibodies to identify these subtle differences in protein structure highlights their usefulness.

Antibodies recognizing the extreme C-terminal region of the OPN molecule (AK3D9 & AK7B4) were also able to inhibit adhesion of a mouse and human cell line to recombinant human OPN. This region of OPN has not been previously implicated as having a role in cell adhesion, and may represent the binding site of the CD44 receptor to the C-terminal thrombin fragment of OPN, which has not been localized [Weber et al., 1996; Katagiri et al., 19991. The AK3D9 binding region on OPN has been shown to be differentially phosphorylated depending on the cell line [Christensen et al., 2007], suggesting that phosphorylation of this region may have a role in regulating CD44 binding.

Uede and colleagues have developed antibodies raised against defined regions of the OPN backbone; one (1B20) recognizes the same area as the AK3D9 antibody described herein [Kon et al., 2000; Kon et al., 20021. Like AK3D9, 1B20 shows similar differences in its ability to recognize the various forms of OPN contained in human urine. Interestingly, the C-terminal 1B20 antibody was demonstrated to bind OPN produced by transfected CHO cells both before and after acid-phosphatase treatment, suggesting that the C-terminal region of the OPN produced by these cells is not phosphorylated.

Recent work has suggested that different ELISA assays have variable sensitivities for plasma and urine OPN [Kon et al., 2000; Vordermark et al., 2006]. Our results suggest that some of this variability may result from the effects of PTMs on antibody reactivity, as well as changes in protein structure resulting from proteolytic cleavage. Our results provide important new reagents to begin to address this complication. Overall this study emphasizes the importance of the heterogenous nature of OPN PTMs, especially when comparing OPN produced by various sources, and stresses the need for careful selection of monoclonal antibodies used for the detection of OPN, particularly when using ELISA systems for quantitation.

In summary, the present inventors have successfully generated a panel of monoclonal antibodies immunologically specific for osteopontin using the knock-out mice of the invention. Two approaches have been utilized. Antibodies have been raised against murine GST-tagged osteopontin. Clones secreting these antibodies have been designated AKMZA1 (also referred to herein as AKM2A1), AKM4AG9, AKM2C5. Antibodies have also been raised against human His-tagged osteopontin. Clones secreting these antibodies have been designated AKM1G4, AKM8B3, and AKM10F6. FIG. 22 provides a schematic diagram of the putative binding sites on osteopontin for some of the monoclonal antibodies described in this example. Table 5 lists features of the antibodies described herein.

### REFERENCES FOR EXAMPLES I, II AND III

1. Denhardt D T, Guo X 1993 Osteopontin: a protein with diverse functions. FASEB J 7:1475-1482.
2. Giachelli C M, Schwartz S M, Liaw L 1995 Molecular and cellular biology of osteopontin: potential role in cardiovascular disease. Trends Cardiovasc Med 5:88-95.
3. Butler W T, Ridall A L, McKee M D 1996 Osteopontin. In: Bilezekian, J P, Raisz, L G, Rodan, G A(eds) Principles of Bone Biology, Academic Press, San Diego, pp. 167-181.
4. Reinholt F P, Hultenby K, Oldberg C, Heinegard D 1990 Osteopontin—a possible anchor of osteoclasts to bone. Proc Natl Acad Sci USA 8:4473-4475.
5. McKee M D, Nanci A 1995 Osteopontin and the bone remodeling sequence. Colloidal-gold immunocytochemistry of an interfacial extracellular matrix protein. Ann NY Acad Sci 760:177-189.
6. Sørensen ES, Højrup P, Petersen TE 1995 Post-translational modification of bovine osteopontin: identification of twenty-eight phosphorylation and three O-glycosylation sites. Protein Sci 4:2040-2049.
7. Patarca R, Saavedra R A, Cantor H 1993 Molecular and cellular basis of genetic resistance to bacterial infection: the role of the early T-lymphocyte activation-1/osteopontin gene. Crit. Rev Immunol 13:225-246.
8. Smith J H, Denhardt D T 1987 Molecular cloning of a tumor promoter-inducible mRNA found in JB6 mouse epidermal cells: Induction is stable at high, but not at low, cell densities. J Cell Biochem 34:13-22.
9. Craig A M, Bowden G T, Chambers A F, Spearman M A, Greenberg A H, Wright J A, McLeod M, Denhardt D T 1990 Secreted phosphoprotein mRNA is induced during multi-stage carcinogenesis in mouse skin and correlates with the metastatic potential of murine fibroblasts. Int J Cancer 46:133-137.
10. Gardner, H A R, Berse B, Senger D F 1994 Specific reduction in osteopontin synthesis by antisense RNA inhibits the tumorigenicity of transformed Rat1 fibroblasts. Oncogene 9:2321-2326.
11. Feng B, Rollo E E, Denhardt D T 1995 Osteopontin (OPN) may facilitate metastasis by protecting cells from macrophage NO-mediated cytotoxicity: evidence from cell lines down-regulated for OPN expression by a targeted ribozyme. Clin Exp Metastasis 13:453-462.
12. Oates A J, Barraclough R, Rudland PS 1996 The identification of osteopontin as a metastasis-related gene product in a rodent mammary tumour model. Oncogene 13:97-104.
13. Chellaiah M, Fitzgerald C, Filardo E J, Cheresh D A, Hruska K A 1996 Osteopontin activation of c-src in human melanoma cells requires the cytoplasmic domain of the integrin αᵥ-subunit. Endocrinology 137:2432-2440.
14. Chellaiah, M, Hruska K 1996 Osteopontin stimulates gelsolin-associated phosphoinositide levels and phosphatidylinositol triphosphate-hydroxyl kinase. Mol Biol Cell 7:743-753.
15. Hwang, S-m, Lopez C A, Heck D E, Gardner C R, Laskin D L, Laskin J D, Denhardt D T 1994 Osteopontin inhibits induction of nitric oxide synthase activity by inflammatory mediators in mouse kidney epithelial cells. J Biol Chem 269:711-715.
16. Rollo E E, Denhardt D T 1996 Differential effects of osteopontin on the cytotoxic activity of macrophages from young and old mice. Immunology 88:642-647.
17. Rollo E E, Laskin D L, Denhardt D T 1996 Osteopontin inhibits nitric oxide production and cytotoxicity by activated RAW264.7 macrophages. J Leukoc Biol 60:397-404.
18. Denhardt D T, Chambers A F 1994 Overcoming obstacles to metastasis—Defenses against host defenses: Osteopontin (OPN) as a shield against attack by cytotoxic host cells. Cell Biochem 56:48-51.
19. Carlson I, Tognazzi K, Manseau E J, Dvorak H F, Brown L F 1997 Osteopontin is strongly expressed by histiocytes in granulomas of diverse etiology. Lab Invest 77:103-108.
20. Nau G J, Guilfoile P, Chupp G F, Berman J S, Kim S J, Kornfeld H, Young R A 1997 A chemoattractant cytokine associated with granulomas in tuberculosis and silicosis. Proc Natl Acad Sci USA 94:6414-6419.
21. Liaw L, Almeida M, Hart C E, Schwartz S M, Giachelli C M 1994 Osteopontin promotes vascular cell adhesion and spreading and is chemotactic for smooth muscle cells in vitro. Circ Res 74:214-224.
22. Senger D R, Ledbetter S R, Claffey K P, Papadopoulos-Sergiou A, Perruzzi C A, Detmar M 1996 Stimulation of endothelial cell migration by vascular permeability factor/vascular endothelial growth factor through cooperative mechanisms involving the αᵥβ₃ integrin, osteopontin, and thrombin. Am J Pathol 149:293-305.
23. Singh R P, Patarca R, Schwartz J, Singh P, Cantor H 1990 Definition of a specific interaction between the early T lymphocyte activation 1 (ETA-1) protein and murine macrophages in vitro and its effect upon macrophages in vivo. J Exptl Med 171:1931-1942.
24. Weber G F, Ashkar S, Glimcher M J, Cantor H 1996 Receptor-ligand interaction between CD-44 and osteopontin (ETA-1). Science (Wash DC) 271:509-512.
25. Giachelli, C M, Pichler R, Lombardi D, Denhardt D T, Alpers C E, Schwartz S M, Johnson R J 1994 Osteopontin expression in angiotensin II-induced tubulointerstitial nephritis. Kidney Int 45:515-524.
26. Pichler R, Giachelli C M, Lombardi D, Pippin J, Gordon K, Alpers C E, Schwartz S M, Johnson R J 1994 Tubulointerstitial disease in glomerulonephritis. Potential role of osteopontin (uropontin). Am J Path 144:915-926.
27. Diamond J R, Kees-Folts D, Ricardo S D, Pruznak A, Eufemio M 1995 Early and persistent up-regulated expression of renal cortical osteopontin in experimental hydronephrosis. Am J Pathol 146:1455-1466.
28. Lopez C A, Hoyer J R, Wilson P D, Waterhouse P, Denhardt D T 1993 Heterogeneity of osteopontin expression among nephrons in mouse kidney and enhanced expression in sclerotic glomeruli. Lab Invest 69:355-363.
29. Kohri K, Nomura S, Kitamura Y, Nagata T, Yoshioka K, Iguchi T, Yamate T, Umekawa Y, Suzuki H, Sinohara H, Kurita T 1993 Structure and expression of the mRNA encoding urinary stone protein (osteopontin). J Biol Chem 258:15180-15184.
30. McKee M D, Nanci A, Khan S R 1995 Ultrastructural immunodetection of osteopontin and osteocalcin as major matrix components of renal calculi. J Bone Miner Res 10:1913-1929.
31. McKee M D, Nanci A 1996 Osteopontin at mineralized tissue interfaces in bone, teeth, and osseointegrated implants: ultrastructural distribution and implications for mineralized tissue formation, turnover, and repair. Microsc Res Tech 33:141-164.
32. McKee M D, Nanci A 1996 Secretion of osteopontin by macrophages and its accumulation at tissue surfaces during wound healing in mineralized tissues: a potential requirement for macrophage adhesion and phagocytosis. Anat Rec 245:394-409.
33. McKee M D, Nanci A 1996 Osteopontin: An interfacial extracellular matrix protein in mineralized tissues. Connect Tissue Res 35:197-205.
34. Boskey A L, Maresca M, Ullrich W, Doty S B, Butler W T, Prince C W 1993. Osteopontin-hydroxyapatite interactions in vitro: inhibition of hydroxyapatite formation and growth in a gelatin-gel. Bone Miner. 22:147-159.
35. Hunter G K 1996 Interfacial aspects of biomineralization. Curr Opin Solid State Mater Science 1:430-435.
36. Hunter G K, Hauschka P V, Poole A R, Rosenberg L C, Goldberg H A 1996 Nucleation and inhibition of hydroxyapatite formation by mineralized tissue proteins. Biochem J 317:59-64.
37. Craig A M, Denhardt D T 1991 The murine gene encoding secreted phosphoprotein 1 (osteopontin): promoter structure, activity, and induction in vivo by estrogen and progesterone. Gene 100:163-171.
38. Thomas K R, Capecchi M R 1987 Site-directed mutagenesis by gene targeting in mouse embryo-derived stem cells. Cell 51:503-512.
39. Mansour S L, Thomas K R, Capecchi M R 1988 Disruption of the proto-oncogene int-2 in mouse embryo-derived cells: a general strategy for targeting mutations to non-selectable genes. Nature (Lond) 336:348-352.
40. Soriano P, Montgomery C, Geske R, Bradley A 1991 Targeted disruption of the c-src proto-oncogene leads to osteopetrosis in mice. Cell 64:693-702.
41. Prince C W, Oosawa T, Butler W T, Tomana M, Bhown A S, Bhown M, Schrohenloher R E 1987 Isolation, characterization, and biosynthesis of a phosphorylated glycoprotein from rat bone. Biol Chem 262:2900-2907.
42. Rittling S R, Novick K E 1997 Osteopontin expression in mammary gland development and tumorigenesis. Cell Growth Differn 8:1061-1069.
43. Fisher L W, Hawkins G R, Tuross N, Termine J D 1987 Purification and partial characterization of small proteoglycans I and II, bone sialoproteins I and II, and osteonectin from the mineral compartment of developing human bone. J. Biol. Chem. 262:9702-9708.
44. Bendayan M 1995 Colloidal gold post-embedding immunocytochemistry. Progr. Histochem. Cytochem. 29:1-159.
45. Takahashi N, Akatsu A, Udagawa N, Sasaki T, Yamaguchi A, Moseley J, Martin T J, Suda T 1988 Osteoblastic cells are involved in osteoclast formation. Endocrinology 123:2600-2602.
46. Udagawa N, Takahashi N, Akatsu T, Sasaki T, Yamaguchi A, Kodama H, Martin T J, Suda T 1989 The bone marrow-derived stromal cell lines MC3T3-G2/PA6 and ST2 support osteoclast-like cell differentiation in cocultures with spleen cells. Endocrinology 125:1805-1813.
47. Matsumoto H N, Tamura M, Denhardt D T, Obinata M, Noda M 1995 Establishment and characterization of bone marrow stromal cell lines that support osteoclastogenesis. Endocrinology 136:4084-4091.
48. Franzen A, Heineg\dot{a}rd D 1985 Isolation and characterization of two sialoproteins present only in bone calcified matrix. Biochem J 232:715-724.
49. Akatsu T, Tamura, T, Takahashi N, Udagawa N, Tanaka S, Sasaki T, Yagamushi A, Nagata N, Suda T 1992 Preparation and characterization of a mouse osteoclast-like multinunucleated cell population. J. Bone Min. Res. 7:1297-1306.
50. Grigoriadis A E, Wang Z Q, Cecchini M G, Hofstetter W, Felix R, Fleisch H A, Wagner E F 1994 c-Fos: a key regulator of osteoclast-macrophage lineage determination and bone remodeling. Science (Wash DC) 266:443-448.
51. Butler W T 1995 Structural and functional domains of osteopontin. Ann. N.Y. Acad. Sci. 760:6-11.
52. Brown L F, Berse B, Van de Water L I, Papadopoulos-Sergiou A, Peruzzi C A, Manseau E J, Dvorak H R, Senger D R 1992 Expression and distribution of osteopontin in human tissues; widespread association with luminal epithelial surfaces. Mol. Biol. Cell. 3:1169-1180.
53. Chen J, Singh K, Mukherjee B B, Sodek J 1993 Developmental expression of osteopontin (OPN) mRNA in rat tissues: evidence for a role for OPN in bone formation and resorption. Matrix 13:113-120.
54. Shiraga H, Min W, VanDusen W J, Clayman M D, Miner D, Terrell C H, Sherbotie J R, Foreman J W, Przysiecki C, Neilson E G, Hoyer J R 1992 Inhibition of calcium oxalate crystal growth in vitro by uropontin, a new member of the aspartic-acid rich protein superfamily. Proc Natl Acad Sci USA 89:426-430.
55. Worcester E M, Blumenthal S S, Beshensky A M, Lewand D L 1992 The calcium oxalate crystal growth inhibitor protein produced by mouse kidney cortical cells in culture is osteopontin. J Bone and Min Res 7:1029-1036.
56. Zheng, X, Saunders T L, Camper S A, Samuelson L C, Ginsburg D 1995 Vitronectin is not essential for normal mammalian development and fertility. Proc Natl Acad Sci USA 92:12426-12430.
57. Saga Y, Yagi T, Ikawa Y, Sakakura T, Aizawa S 1992 Mice develop normally without tenascin. Genes Dev 6:1821-1831.
58. Goldberg H A, Hunter G K 1995 The inhibitory activity of osteopontin on hydroxyapatite formation in vitro. Ann NY Acad Sci 760:305-308.
59. Moradian-Oldak J, Frolow F, Addadi L, Weiner S 1992 Interactions between acidic matrix macromolecules and calcium phosphate ester crystals: relevance to carbonate apatite formation in biomineralization. Proc R Soc Lond [Biol] 24:47-55.
60. Aizenberg J, Hanson J, Ilan M, Leiserowitz L, Koetzle T F, Addadi L, Weiner S 1995 Morphogenesis of calcitic sponge spicules: a role for specialized proteins interacting with growing crystals. FASEB J. 9:262-268.
61. Yamate T, Mocharla H, Taguchi Y, Igietseme J U, Manolagas S C, Abe E 1997 Osteopontin expression by osteoclast and osteoblast progenitors in the murine bone marrow: Demonstration of its requirement for osteoclastogenesis and its increase after ovariectomy. Endocrinology 138:3047-3055.
62. Young B A, Burdmann E A, Johnson R J, Alpers C E, Giachelli C M, Eng E, Andoh T, Bennett W M, Couser W G 1995 Cellular proliferation and macrophage influx precede interstitial fibrosis in cyclosporine nephrotoxicity. Kidney Int 48:439-438.
63. Lampe M A, Patarca R, Iregui M V, Cantor H 1991 Polyclonal B-cell activation by the Eta-1 cytokine and the development of autoimmune disease. J Immunol 147:2902-2906.
64. Giachelli C M, Lombardi D, Johnson R J, Murry C E, Almeida M. 1998 Evidence for a role of osteopontin in macrophage infiltration in response to pathological stimuli in vivo. Am Pathol 152: 353-358.
65. McKee M D, Nanci A. 1996 Secretion of osteopontin by macrophages and its accumulation at tissue surfaces during wound healing in mineralized tissues: A potential requirement for macrophage adhesion and phagocytosis. Anat Rec 245:394-409.
66. Rodan G 1995 Osteopontin overview. Ann NY Acad Sci 760:1-5.
67. Sørensen E S, Rasmussen L K, Møller L, Jensen P H, Højrup P, Petersen T E 1994 Localization of transglutaminase-reactive glutamine residues in bovine osteopontin. Biochem J 304:13-16.
68. Evans et al., (1981) Nature 292:154-156.
69. Bradley et al., (1984) Nature 309:255-258.
70. Gossler et al., (1986) Proc. Natl. Acad. Sci. 83:9065-9069.
71. Frohman et al., (1989) Cell 56:145-147.
72. Bradley et al., (1992) Bio/Technology 10:534-539.
73. Greendale et al., (1993) J. Am. Geriatr. 41:426-436.
74. Fukushima et al., (1991) Anat. Rec. 231:298-315.
75. Crippes et al., (1994) J. Bone Min. Res. 9: 5178.
76. Nesbitt et al., (11930 Exp. Cell Res. 195:368-375.
77. Yamamoto et al., (1993) J. Bone. Min. Res. 3:S123.
78. Liaw et al., (1998) J. Clinical Investigation 101:1468-1478.
79. Al-Shami R, Sorensen E S, Ek-Rylander B, Andersson G, Carson D D, Farach-Carson M C (2005): Phosphorylated osteopontin promotes migration of human choriocarcinoma cells via a p70 S6 kinase-dependent pathway. J Cell Biochem 94: 1218-33.
80. Chambers A F, Hota C, Prince C W (1993): Adhesion of metastatic, ras-transformed NIH 3T3 cells to osteopontin, fibronectin, and laminin. Cancer Res 53:701-6.
81. Christensen B, Kazanecki C C, Petersen T E, Rittling S R, Denhardt D T, Sorensen ES (2007): Cell-type specific post-translational modifications of mouse osteopontin are associated with different adhesive properties. Journal of Biological Chemistry manuscript:M703055200, epub ahead of print.
82. Christensen B, Nielsen M S, Haselmann K F, Petersen T E, Sorensen E S (2005): Posttranslationally modified residues of native human osteopontin are located in clusters: identification of 36 phosphorylation and five O-glycosylation sites and their biological implications. Biochem J 390:285-92.
83. Crawford H C, Matrisian L M, Liaw L (1998): Distinct roles of osteopontin in host defense activity and tumor survival during squamous cell carcinoma progression in vivo. Cancer Res 58:5206-15.
84. D′Alonzo R C, Kowalski A J, Denhardt D T, Nickols G A, Partridge N C (2002): Regulation of collagenase-3 and osteocalcin gene expression by collagen and osteopontin in differentiating MC3T3-El cells. J Biol Chem 277:24788-98.
85. Declerck P J, Carmeliet P, Verstreken M, De Cock F, Collen D (1995): Generation of monoclonal antibodies against autologous proteins in gene-inactivated mice. J Biol Chem 270:8397-400.
86. Denhardt D T, Giachelli C M, Rittling S R (2001a): Role of osteopontin in cellular signaling and toxicant injury. Annu Rev Pharmacol Toxic01 41:723-49.
87. Denhardt D T, Noda M, O'Regan A W, Pavlin D, Berman J S (2001b): Osteopontin as a means to cope with environmental insults: regulation of inflammation, tissue remodeling, and cell survival. J Clin Invest 107: 1055-61.
88. Dunn I S (1996): Phage display of proteins. Curr Opin Biotechnol 7547-53.
89. Ek-Rylander B, Flores My Wendel M, Heinegard D, Andersson G (1994): Dephosphorylation of osteopontin and bone sialoprotein by osteoclastic tartrate-resistant acid phosphatase. Modulation of osteoclast adhesion in vitro. J Biol Chem 269: 14853-6.
90. Gericke A, Qin C, Spevak L, Fujimoto Y, Butler W T, Sorensen E S, Boskey A L (2005): Importance of Phosphorylation for Osteopontin Regulation of Biomineralization. Calcif Tissue Int 77:45-54.
91. Goodwin A E, Pauli B U (1995): A new adhesion assay using bouyancy to remove non-adherent cells. Journal of Immunological Methods 187:213-19.
92. Harlow E, Lane D (1999): “Using Antibodies: A laloratory manual.” Cold Spring Harbor: Cold Spring Harbor Laboratory Press.
93. Hotta H, Kon S, Katagiri Y U, Tosa N, Tsukamoto T, Chambers A F, Uede T (1999): Detection of various epitopes of murine osteopontin by monoclonal antibodies. Biochem Biophys Res Commun 257:6-11.
94. Hoyer J R, Asplin J R, Otvos L (2001): Phosphorylated osteopontin peptides suppress crystallization by inhibiting the growth of calcium oxalate crystals. Kidney Int 60:77-82.
95. Jono S, Peinado C, Giachelli C M (2000): Phosphorylation of osteopontin is required for inhibition of vascular smooth muscle cell calcification. J Biol Chem 275:20197-203.
96. Katagiri Yu, Sleeman J, Fujii H, Herrlich P, Hotta H, Tanaka K, Chikuma S, Yagita H, Okumura K, Murakarni M, Saiki I, Chambers A F, Uede T (1999): CD44 variants but not CD44s cooperate with beta1-containing integrins to permit cells to bind to osteopontin independently of arginine-glycine-aspartic acid, thereby stimulating cell motility and chemotaxis. Cancer Res 59:219-26.
97. Keykhosravani M, Doherty-Kirby A, Zhang C, Brewer D, Goldberg H A, Hunter G K, Lajoie G (2005): Comprehensive identification of post-translational modifications of rat bone osteopontin by mass spectrometry. Biochemistry 44:6990-7003.
98. Kleinman J G, Wesson JAY Hughes J (2004): Osteopontin and calcium stone formation. Nephron Physiol 98:p43-7.
99. Kon S, Maeda M, Segawa T, Hagiwara Y, Horikoshi Y, Chikuma S, Tanaka K, Rashid M M, Inobe M, Chambers A F, Uede T (2000): Antibodies to different peptides in osteopontin reveal complexities in the various secreted forms. J Cell Biochem 77:487-98.
100. Kon S, Yokosaki Y, Maeda M, Segawa T, Horikoshi Y, Tsukagoshi H, Rashid M M, Morimoto J, Inobe M, Shijubo N, Chambers A F, Uede T (2002): Mapping of functional epitopes of osteopontin by monoclonal antibodies raised against defined internal sequences. J Cell Biochem 84420-32.
101. Kowalski A (2005): Creation, Characterization, and Application of Novel Anti-Osteopontin Monoclonal Antibodies. Doctoral Dissertation, Department of Microbiology and Molecular Genetics. Rutgers University, the State University of New Jersey.
102. Pampena DAY Robertson K A, Litvinova O, Lajoie G, Goldberg H A, Hunter G K (2003): Inhibition of hydroxyapatite formation by osteopontin phosphopeptides. Biochem J. Pt.
103. Razzouk S, Brunn J C, Qin C, Tye C E, Goldberg H A, Butler W T (2002): Osteopontin posttranslational modifications, possibly phosphorylation, are required for in vitro bone resorption but not osteoclast adhesion. Bone 30:40-7.
104. Rittling S R, Matsumoto H N, McKee M D, Nanci A, An X R, Novick K E, Kowalski A J, Noda M, Denhardt D T (1998): Mice lacking osteopontin show normal development and bone structure but display altered osteoclast formation in vitro. J Bone Miner Res 13: 1101-11.
105. Rollo E E (1995): Inhibition of Nitric Oxide Production and Macrophage-Mediated Cytotoxicity by Recombinant Human Osteopontin. Doctoral Dissertation, Department of Microbiology and Molecular Genetics. Rutgers, the State University of New Jersey.
106. Smothers J F, Henikoff S, Carter P (2002): Tech. Sight. Phage display. Affinity selection from biological libraries. Science 298:621-2.
107. Sodek J, Batista Da Silva A P, Zohar R (2006): Osteopontin and mucosal protection. J Dent Res 85:404-15.
108. Slarensen E S, Hojrup P, Petersen T E (1995): Posttranslational modifications of bovine osteopontin: identification of twenty-eight phosphorylation and three O-glycosylation sites. Protein Sci 4:2040-9.
109. Vordermark D, Said H M, Katzer A, Kuhnt T, Hansgen G, Dunst J, Flentje M, Bache M (2006): Plasma osteopontin levels in patients with head and neck cancer and cervix cancer are critically dependent on the choice of ELISA system. BMC Cancer 6:207.
110. Weber G F, Ashkar S, Glimcher M J, Cantor H (1996): Receptor-ligand interaction between CD44 and osteopontin (Eta-1). Science 271:509-12.
111. Wu Y, Denhardt D T, Rittling S R (2000): Osteopontin is required for full expression of the transformed phenotype by the ras oncogene. Br J Cancer 83: 156-63.
112. Young M F, Ken J M, Termine J D, Wewer U M, Wang M G, McBride O W, Fisher L W (1990): cDNA cloning, mRNA distribution and heterogeneity, chromosomal location, and RFLP analysis of human osteopontin (OPN). Genomics 7:491-502.
113. Zhu B, Suzuki K, Goldberg H A, Rittling S R, Denhardt D T, McCulloch C A, Sodek J (2004): Osteopontin modulates CD44-dependent chemotaxis of peritoneal macrophages through Gprotein-coupled receptors: evidence of a role for an intracellular form of osteopontin. J Cell Physiol 198: 155-67.
114. Zohar R, Lee W, Arora P, Cheifetz S, McCulloch C, Sodek J (1997): Single cell analysis of intracellular osteopontin in osteogenic cultures of fetal rat calvarial cells. J Cell Physiol 170:88-100.

### Example IV

### Plasma Osteopontin Modulates Chronic Restraint Stress-Induced Thymus Atrophy by Regulating Stress Hormones

### Inhibition by an Anti-OPN Monoclonal Antibody

Osteopontin (OPN) also acts as a cytokine implicated in mediating responses to certain stressors, including mechanical, oxidative and cellular stress. However, the involvement of OPN in responding to other physical and psychological stress applied to the intact animal is largely unexplored. Our previous research revealed that OPN is critical for hindlimb-unloading-induced lymphoid organ atrophy through modulation of corticosteroid production. In the present example, we demonstrate that OPN^{-/-} mice are resistant to chronic restraint stress (CRS)-induced lymphoid (largely thymus) organ atrophy; additionally, the stress-induced up-regulation of corticosterone production is significantly reduced in OPN mice. Underlying this observation is the fact that normal adrenocorticotropic hormone levels are substantially reduced in the OPN^{-/-} mice. Our data demonstrate both that injection of OPN into OPN-deficient mice enhances the CRS-induced lymphoid organ atrophy and that injection of a specific anti-OPN monoclonal antibody (2C5) into wild type mice ameliorates the CRS-induced organ atrophy; changes in corticosterone levels were also partially reversed. These studies reveal that OPN plays a significant role in the regulation of the hypothalamus-pituitary-adrenal axis hormones and that it augments CRS-induced organ atrophy.

Osteopontin (OPN) is a pleiotropic phosphoglycoprotein that is broadly expressed and upregulated during inflammation, autoimmune diseases, cancer development, and various stress conditions (reviews: 1-3, Denhardt et al., 2001; Sodek et al., 2006; Scatena et al., 2007). It interacts with different cell-surface receptors, including integrins and certain CD44 isoforms and can induce phosphoinositide-3-kinase/Akt-dependent NF-KB activation (reviews: 4, Wang and Denhardt, 2008). It is difficult to determine the molecular mechanism of a specific effect of OPN due to the interplay with various factors including its ability to engage multiple integrins and CD44 variants; its posttranslational modifications; its cleavage state; and its localization both intracellularly and extracellularly. OPN has important cytokine and chemokine functions and is a key stress mediator (4, Wang and Denhardt, 2008). Its roles in mediating oxidative stress (5 Itoh et al., 2005), mechanical stress (6 Fujihara et al., 2006) and cellular stress (7 Wai and Kuo, 2004) have been well documented. We have demonstrated that OPN is at least partially responsible for hind-limb unloading (HU) stress-induced losses in peripheral lymphocytes and thymocytes (8 Wang et al., 2007). This type of stress leads to rapid systemic changes in stress hormone production, immune cell distribution, and cytokine/chemokine production; it also affects peripheral immune organs in the immune system (9 Sonnenfeld, 2005). OPN-deficient mice showed significantly milder changes in response to this stress. However, the extent to which each of these different stress paradigms affects the HPA (hypothalamus-pituitary-adrenal) axis and the immune system remains to be determined.

Another murine stress model, chronic restraint stress (CRS), has been widely used in studies of the effect of stress hormones (10 Nacher et al., 2004) and immune cell functions in mice (11, 12 Yin et al., 2000; Zhang et al., 2008). CRS consists of a scheduled confinement and restriction of food and water during restraint. In addition to physical immobilization, psychological stress plays a significant part in this model (13 Bowers et al., 2008). We used this model to evaluate OPN-deficient mice in both 129 and Balb/c backgrounds to determine their stress response as assessed by lymphoid organ atrophy, changes in corticosterone (CORT) and adrenocorticotropic hormone (ACTH) levels, and leukocyte trafficking as compared to their wild type counterparts.

To further verify the specific role of OPN in the lymphocyte stress response and its effect on HPA axis hormones, we used mouse fibroblast-derived OPN and anti-OPN monoclonal antibodies to evaluate respectively the ability of OPN to restore the wild type phenotype to OPN^{-/-} mice or the effect of depletion of OPN with anti-OPN monoclonal antibodies in inhibiting the stress-induced lymphoid organ atrophy and associated hormonal changes in WT mice. Our results show that exogenously supplied OPN can sensitize OPN^{-/-} mice to CRS-induced thymus atrophy, demonstrating a critical role of OPN of organ atrophy. On the other hand, wild type mice that received the monoclonal antibody 2C5 exhibited a significant protection from CRS-induced lymphoid organ atrophy. These results support the conclusion that plasma OPN regulates stress-induced organ atrophy and demonstrate the involvement of OPN in the bidirectional communication between the central nervous system and the immune system.

The following materials and methods are provided to facilitate the practice of Example IV.

### Animals.

OPN^{-/-} mice in the 129 background were generated as described in Example I (27, 28 Rittling et al., 1998, Natasha et al., 2006) and maintained along with isogenic wild type controls in the Rutgers Nelson Animal Facility, which is accredited by the Association for Assessment and Accreditation of Laboratory Animal Care and is under the care of a board-certified veterinarian. The research with these mice was approved by the Rutgers Institutional Animal Care and Use Committee, protocol number 97:031.

OPN^{-/-} mice on the Balb/c background were kindly provided by Drs. Mari Shinohara and Harvey Cantor (29 Shinohara et al., 2006). The breeding pairs were homozygous OPN^{-/-} mice with 15 generations of backcrossing to Balb/c background therefore considered 99.99% pure Balb/c background. Breeding pairs were bred and maintained in the Rutgers Nelson Animal Facility as above. Balb/c wild type control mice were purchased from the Jackson Laboratory (Bar Harbor, Me.). All animals used in the experiments were age- and sex-matched.

### Immuno-Affinity Purification of Osteopontin

Mouse OPN was purified from serum-free medium conditioned by a ras-transformed murine embryonic fibroblast line (275-3-2) (30 Wu et al., 2000). The medium was incubated with 1 ml of protein G beads (Pierce, Rockford, Ill.) to which the 2A1 anti-OPN monoclonal antibody had been cross linked. The beads were washed and packed into a 2-ml disposable column. OPN was eluted from the 2A1-protein G beads with 100 mM glycine, 500 mM NaCl, pH 2.5 and collected into tubes containing a neutralizing pH 8 Tris buffer. Fractions were analyzed by SDS-PAGE and proteins visualized by non-ammoniacal silver staining and western blotting. Positive fractions were pooled, desalted on PD-10 columns (GE Healthcare Bio-Sciences, Piscataway, N.J.), quantified by ELISA and lyophilized.

### Monoclonal Anti-OPN Antibodies

The monoclonal anti-osteopontin hybridomas used [mAK2A1 (2A1), mAK3D9 (3D9), mAK1G4 (1G4) and mAK2C5 (2C5)] were generated and characterized by Dr. Aaron Kowalski (16 Kowalski, 2005). Antibodies were purified from ascites fluid obtained from mice injected with the different hybridomas in the laboratory of Dr. Yacov Ron (Robert Wood Johnson Medical School, University of Medicine and Dentistry of New Jersey).

### Chronic Restraint Stress (CRS)

Eight to ten-week-old mice were subjected to an established CRS protocol with some modification (11 Yin et al., 2000). Briefly, OPN^{+/+} and OPN^{-/-} mice were each divided into control and stress groups. Mice used in a study were randomized by evenly distributing mice from the same litter to different treatment groups so as to minimize the influence of litter and age variations. Mice were immobilized individually in well-ventilated cylindrical wire mesh restrainers sized 12 cm (length)×3 cm (diameter) that were clamped on both ends. The restrained mice were held horizontally in their home cages during the restraint sessions. They were restrained for 12 h daily followed by a 12 h recovery. Food and water were provided during the recovery period ad libitum. Control animals were undisturbed in their home cages or (when appropriate) injected with PBS to control for antibody or OPN injections. Six mice were used in each treatment group whenever possible or the data from parallel experiments were combined for the statistical analysis. Balb/c and 129 mice were restrained for 2 and 3 12-h periods respectively.

At the end of the final restraint stress session, animals were euthanized by CO₂ inhalation and the blood, spleen, and thymus harvested. Blood was drawn immediately after euthanasia by cardiac puncture. Approximately 0.5-0.8 ml blood was collected from each mouse and mixed with 50 μl of 50 mM EDTA in chilled PBS (anticoagulant). Plasma samples were collected by centrifugation at 4° C. for 15 min at 10,000 rpm in a microcentrifuge. Supernatants were removed and stored at −80° C. The spleen and thymus were excised and put into 1 ml of cell culture medium for preservation. The weight of each organ was recorded.

### Administration of Mouse Fibroblast-derived OPN

Purified mouse fibroblast OPN was re-hydrated from lyophilized stock and diluted in sterile PBS before use. This OPN is phosphorylated randomly at a few serine/threonine sites (out of some 30 potential sites) and presumably behaves as un-phosphorylated OPN (31 Christensen et al., 2005). OPN^{-/-} mice were divided into 3 groups: (A) control group, (B) CRS group injected with PBS, and (C) CRS group injected with OPN in PBS. Mice in Group (C) were injected intraperitoneally daily with 5 μg of OPN in 100 μl of PBS starting 3 days before the first restraint cycle and continued through the restraint sessions. Mice received 25-30 μg OPN by the end of treatment depending on the number of restraint cycles. The mice in group (B) received 100 μl of PBS, using the same schedule as group (C), and were similarly restrained. Mice in control group (A) were kept in their home cages undisturbed.

### Administration of Anti-OPN Monoclonal Antibodies

The monoclonal antibodies were diluted in sterile PBS to 0.66 μg/μl Wild type OPN^{+/+} mice were divided into the (A) control group, (B) CRS group injected with PBS, and (C) CRS group injected with anti-OPN mAb in PBS. Mice in Group (C) were injected with 100 μg of anti-OPN mAb in 150 μl of PBS i.p. starting 24 h before the first restraint cycle and then immediately before each restraint session. The total amount of anti-OPN mAb received at the end of treatment was 300-400 μg depending on the number of restraint cycles. CRS group (B) received 150 μl of PBS and was subjected to restraint as group (C). Control group (A) mice were kept in their home cages undisturbed.

### Measurement of CORT and ACTH in Plasma

The levels of CORT in plasma samples were assessed with a CORT ELISA kit from IBL America (Minneapolis, Minn., Cat# RE52211) according to the manufacturer's instructions. The levels of ACTH were measured using an ACTH ELISA kit from MDbiosciences (St Paul, Minn., Cat# ACTH.96) according to the manufacturer's instructions.

### Determination of OPN Levels in Plasma

High-binding ELISA plates were coated with 0.8 μg/ml anti-OPN Ab (R&D Systems, AF808) in PBS overnight at 4° C. Coated wells were blocked with 1% BSA, 5% sucrose in PBS and incubated for 1 h before samples were applied to wells. Plasma samples were diluted 1:100 in assay diluent (PBS+1% BSA) and 100 μl of diluted samples were added to the wells. After 2 h incubation at room temperature, the plate was washed and detection was performed by incubating the plate with 100 μl of biotinylated anti-OPN mAb at 0.1 μg/ml (BAF808, R&D systems) at room temperature for 2 hours. After the plate was washed, a secondary detection reagent, 100 μl of streptavidin-HRP (1:200 dilution, DY998, R&D systems) was added to the plate and incubated for 20 min. For color development, 100 μl of 3,3′,5,5′-tetramethylbenzidine (TMB) liquid substrate system (T8665, Sigma, St Louis, Mo.) was added to the washed plate and incubated for 15-20 min. Color development was terminated with 50 μl of the stop solution and absorbance was determined by a spectroMax microplate reader (Molecular Devices, Sunnyvale, Calif.) at 450 nm. Recombinant mouse OPN (441-0P, R&D systems) was used as a protein standard in the OPN ELISA. The assays were carried out in triplicate.

### Analysis of Immune Cell Populations

Blood: after removal of the plasma as described above, the pellet material was mixed with 1 ml of red blood cell lysing buffer (R7757, Sigma, St Louis, Mo.) and incubated on ice for 5 min to lyse the red blood cells. The mixture was then diluted in 10 ml of PBS and spun at 1000 rpm for 5 min. The white blood cell pellets were washed with 10 ml PBS again and then resuspended in 100 μl of PBS+2% FBS.

Spleen and Thymus: single-cell suspensions were prepared by grinding the tissue with a syringe plunger and passing through a 70-μm cell strainer. Red blood cells were lysed by adding 1 ml of the red blood cell lysing buffer (Sigma, St Louis, Mo.) to the cell pellet and incubating on ice for 5 min. Cell lysis was terminated by adding 10 vol of PBS to the cells and centrifuging at 1000 rpm for 5 min. Remaining white cells were washed again and resuspended in PBS+2% FBS.

Cells from blood, spleen and thymus were processed in parallel for labeling and analysis. Specific lymphocyte subpopulations and granulocytes were identified on the basis of cell surface markers by flow cytometry: fluorescence-conjugated monoclonal antibodies including rat anti-mouse CD4 (clone RM4-5), CD8 (clone 53-6.7), and CD45R/B220 (clone RA3-6B2) (all from BD Biosciences-PharMingen, San Diego Calif.) were used. Cells were incubated with BD Fc Block (clone 2.4G2) (BD Biosciences-PharMingen, San Diego Calif.) for 10 min to block non-specific binding and then incubated with specific mAbs for 20 min on ice, washed with PBS and analyzed on a multicolor flow cytometer (FACScalibur, Becton Dickinson, San Jose, Calif.). Data were acquired and analyzed with CellQuest software (Becton Dickinson, San Jose, Calif.).

### Results

Lymphoid Organ Atrophy in OPN^{+/+} and OPN^{-/-} Mice after Chronic Restraint Stress

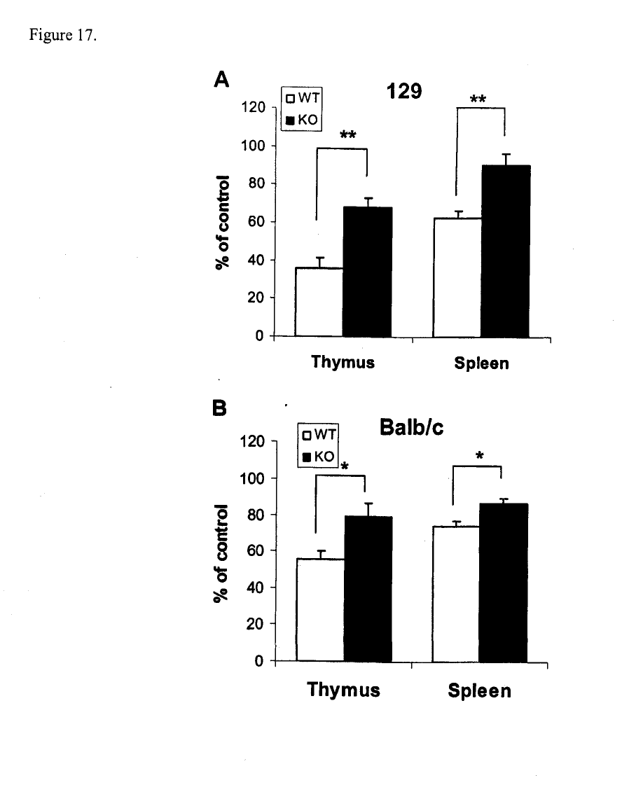

To verify the involvement of OPN in the stress response revealed in our previous research using the HU model (8, Wang et al., 2007), we employed the chronic stress model (CRS) (11, Yin et al., 2000). To reduce the possibility that differences in genetic background could affect the response to stress, OPN^{-/-} mice in a Balb/c background were also tested in parallel with 129 mice. When subjected to CRS, the wild type and OPN-deficient Balb/c mice exhibited statistically significant 8.0% and 4.8% reductions in body weight respectively (p=0.046, 14 Wang, 2008). As shown in FIG. 17A, CRS caused a 60% reduction in thymus weight in 129 OPN^{+/+} mice but only a 30% reduction in the OPN-deficient mice. A 40% reduction of spleen weight was observed in WT mice compared to a 10% reduction in OPN^{-/-} mice; similar responses were observed using Balb/c mice (FIG. 17B). These results indicated that CRS caused lymphoid organ atrophy in both Balb/c and 129 wild type mice to a significantly greater extent than in OPN^{-/-} mice. This confirms that OPN indeed plays a role in mediating stress-induced responses in immune organs.

### Stress-induced Changes in HPA Axis Hormones are Affected by OPN

Because OPN has been found to regulate corticosterone production in response to HU stress (8 Wang et al., 2007), we were interested in discovering whether OPN directly affects corticosterone production or acts by regulating upstream hormones in the HPA axis. Corticosteroid secretion occurs in a circadian pattern and in response to stress. Corticosteroid also provides negative feedback regulation to other stress hormones in the HPA axis by inhibiting the secretion of ACTH and CRH (21 Keller-Wood and Dallman, 1984). Thus in addition to evaluating the corticosterone levels, we evaluated plasma levels of ACTH in order to more closely localize where OPN interacts with the HPA axis.

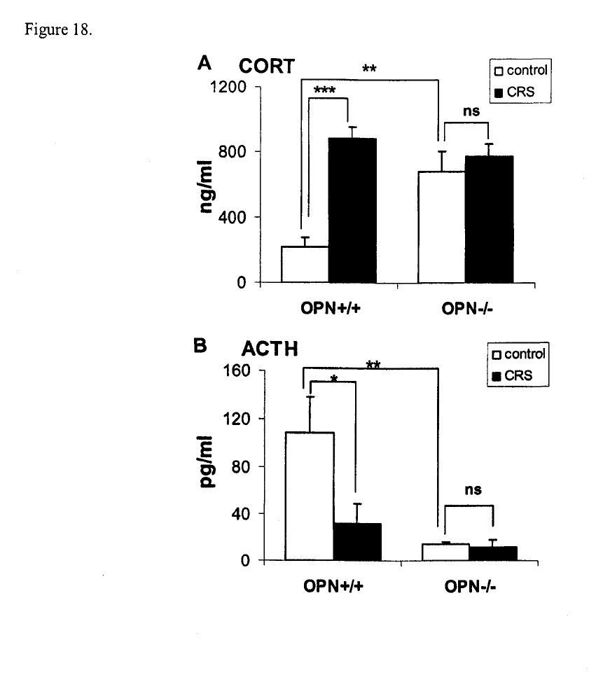

The levels of corticosterone, CRH and ACTH in blood samples harvested immediately after the termination of CRS were tested with commercial ELISA kits. Results showed that the level of corticosterone was highly up-regulated in stressed WT mice. However, in KO mice, there was no significant difference in corticosterone levels between control (un-stressed) and stressed mice (FIG. 18A). Interestingly, the basal level of corticosterone in unstressed mice was significantly higher in KO mice, implying that OPN plays a role in controlling the production of corticosterone; in the absence of OPN, corticosteroid production is apparently elevated leading to a persistent high level of corticosterone in circulation in the absence of applied stress. On the other hand, classic negative feedback mechanism of CORT towards ACTH in response to chronic stress suggests that upregulation of CORT could lead to a reduction of ACTH. The results from the ACTH ELISA assay reflect this reciprocal relationship by showing that the levels of ACTH in WT mice were higher in control mice but largely suppressed in the stressed mice (FIG. 18B). However, in the OPN^{-/-} mice, the ACTH levels were very low and not respond to CRS, suggesting that ACTH secretion was inhibited by the persistent high level of CORT in the OPN^{-/-} mice. Nevertheless, these results demonstrated a critical role of OPN in regulation of HPA axis function.

### OPN Modulates Stress-affected Immune Cell Populations in Different Immune Compartments

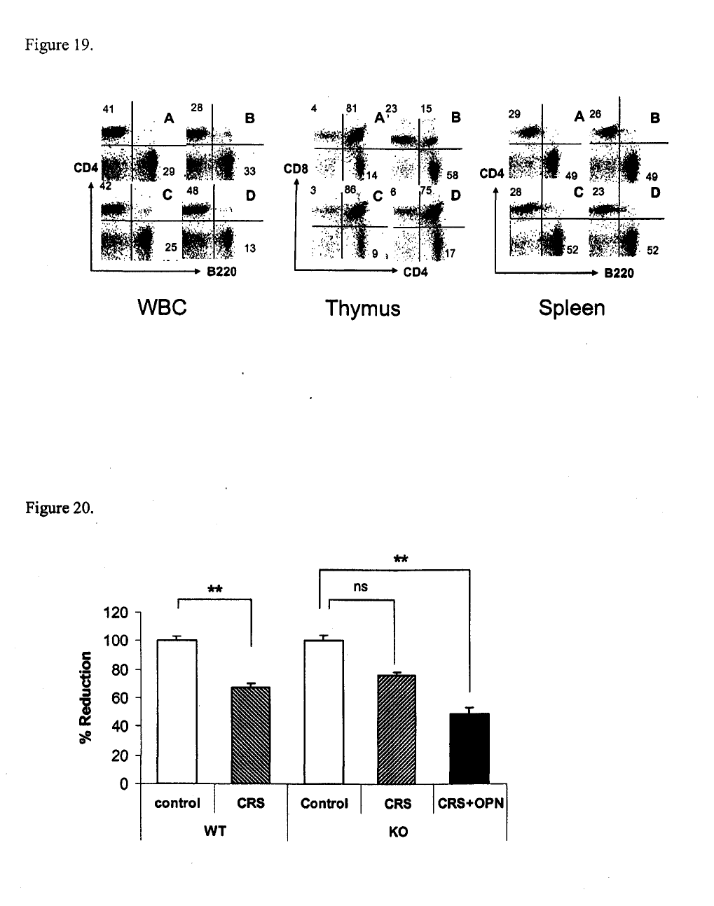

To determine whether OPN contributes to stress-induced immune cell homeostasis, lymphocyte populations in the blood, spleen, and thymus were examined by flow cytometry. As shown in FIG. 19A, the percent of CD4⁺ T cells was significantly decreased in the blood of WT mice after CRS, indicating that stress led to a reduction in CD4⁺ T cells. However in the OPN^{-/-} mice, the levels of CD4⁺ T cells in this compartment was not altered. Interestingly, the percent of B cells in the blood exhibited an opposite trend in response to stress in OPN^{+/+} and OPN^{-/-} mice. While B cells increased in blood of WT mice after CRS, they decreased in the OPN^{-/-} mice. Although in mice subjected to HU (8 Wang et al., 2007), all 3 major lymphocyte populations (B cells, CD4⁺, CD8⁺T cells) in spleen were significantly reduced, resulting in a dramatic reduction in the mass of the spleen; in mice subjected to CRS, only CD4+T cells were affected significantly. This may explain the observed weak or insignificant spleen atrophy in CRS experiments. Furthermore, all three lymphocyte populations in spleen were not affected by CRS in OPN^{-/-} mice (8 Wang, 2008).

As determined before by its weight alone, the thymus is the organ most affected by stress showing a dramatic reduction of mass after CRS. The results from cell type profiling are consistent and confirmed this conclusion. CRS caused a statistically significant reduction in total lymphocytes numbers in both OPN^{+/+} and OPN^{-/-} mice, but the degree of reduction was more dramatic in WT mice than in OPN^{-/-} mice (FIG. 19B). Thymus tissue of wild type mice consists of about 80% double positive (DP) T cells (CD4⁺/CD8⁺), therefore the reduction in the DP population has the greatest impact on the total mass of the organ. These data correlate with the organ weight results and identify the cell types contributing to the organ atrophy.

### Exogenous OPN Causes Stress-induced Lymphocyte Atrophy in OPN^{-/-} Mice

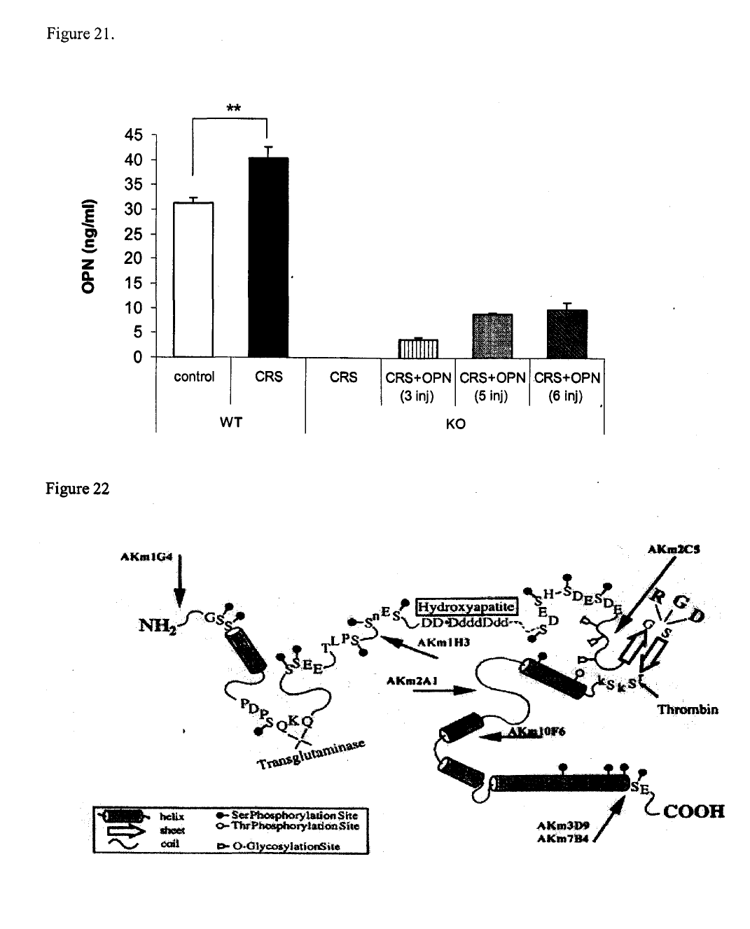

To demonstrate directly that stress-induced organ atrophy is promoted by the presence of OPN, purified mouse OPN produced by ras-transformed mouse fibroblasts was injected into OPN mice prior to and during CRS. OPN^{-/-} mice were injected intraperitoneally with 5 μg of OPN daily for three days prior to subjecting to restraint. Similar injections were made at the beginning of each 24-h restraint cycle to maintain levels of exogenous OPN in the circulation during the restraint. As shown in FIG. 20, CRS led to a larger reduction of thymus weight (38%, p=0.016) in Balb/c OPN^{-/-} mice compared to OPN^{-/-} mice (27%, p=0.078). Elevation of plasma OPN levels by repeated intraperitoneal injections of OPN into Balb/c OPN^{-/-} mice resulted in a larger (51%, p=0.021) reduction of thymus weight. These results have been closely reproduced in several other experiments using OPN^{-/-} mice in the 129 background (20, data not shown). To confirm the presence of OPN circulating in the blood during this experiment, plasma samples harvested at the end of the experiments were assayed for OPN levels. FIG. 21 shows that OPN levels were up-regulated by CRS by about 25% in wild type mice. In OPN knockout mice, as expected, OPN was undetectable. OPN was detectable in the plasma of all samples from the experiments in which exogenous OPN was supplied to OPN^{-/-} mice. The concentration of OPN correlated to the number of injections each animal received. Animals receiving a total of 5 injections (25 μg) had twice as much OPN in the plasma compared to animals receiving 3 injections. However, even with up to 6 injections, the OPN level in the plasma reached only 30% of the wild type level. Nevertheless, these results clearly demonstrated that OPN in the plasma is essential for promoting stress-induced lymphoid organ atrophy.

### An Anti-OPN Monoclonal Antibody Inhibits Stress-induced Organ Atrophy in OPN^{+/+} Mice

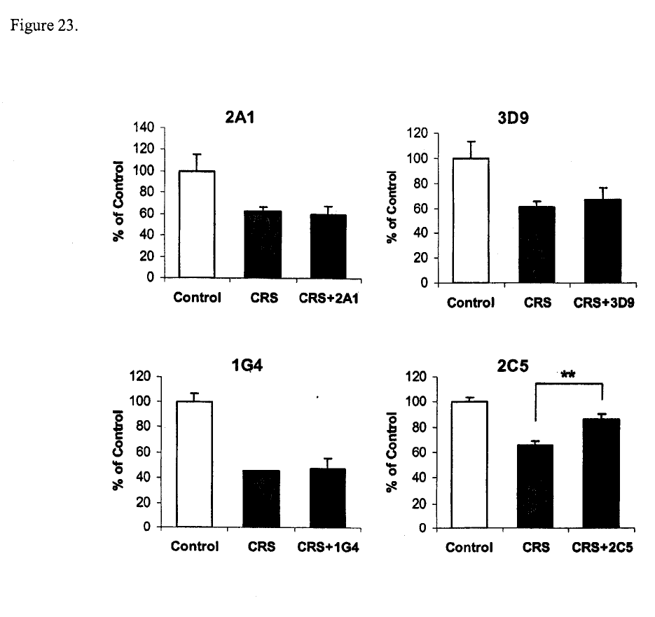

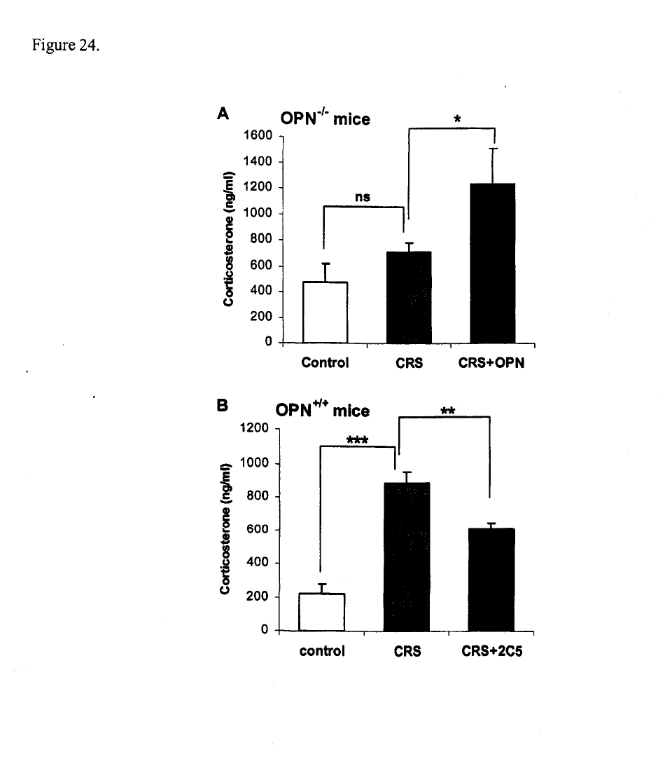

It was encouraging to find that OPN can restore partially the wild type phenotype of stress-induced organ atrophy in OPN^{-/-} mice. The other side of the coin is whether sequestering of OPN in wild type mice would protect the lymphoid organs from stress-induced atrophy. To address this question, 4 different monoclonal antibodies (2A1, 3D9, 2C5, 1G4) (18 Kowalski, PhD thesis 2005; 22 Kazanecki et al., 2007) were evaluated for their effectiveness in preventing stress-induced thymus atrophy in wild type mice. Each mAb recognizes a distinct epitope (Kowalski, 2005; Kazanecki, 2007) on the OPN molecule as indicated in FIG. 22. Wild type Balb/c or 129 OPN mice were injected with 100 μg of mAb 24 hours before starting the CRS cycle and again at the beginning of each restraint cycle. Of the four monoclonal antibodies tested, only 2C5 supported a statistically significant change in thymus weight compared to CRS only group (FIG. 23). Injection of 2C5 blocked stress-induced reduction in thymus weight to the level of that in the OPN^{-/-} mice after CRS (FIG. 24). The other three anti-OPN mAbs were without effect. Two additional experiments yielded similar results. Our recent research has confirmed that 2C5 recognizes a sequence amino terminal to the RGD integrin binding site and may block the interaction of OPN with integrins, though this last remains to be confirmed. Since mAbs recognizing other regions of the OPN molecule tested were not effective, this result suggests that an integrin interaction may be important for OPN function in mediating the stress response.

When OPN levels in mice injected with an anti-OPN mAb were measured, an increase of OPN (how much) was detected (data not shown, 14 Wang PhD thesis 2008). It is known that while antibody binding to the target protein may inactivate the function of that protein by blocking a functional site or by reducing the free state of the target protein, it can also inhibit the turnover of the protein, thereby causing the accumulation in the plasma of the target protein. Nevertheless, administration of 2C5 moderately reversed the severe thymus atrophy caused by stress.

Injection of OPN into OPN^{-/-} mice elevated corticosterone levels in the plasma of the mice after CRS (FIG. 24A). These results indicate that exogenous OPN supplied to OPN^{-/-} mice partly restores the wild type phenotype; the presence of OPN in the circulation may modulate stress hormones and other unknown factors to cause the increased organ atrophy in response to stress. To determine whether an anti-OPN mAb injection could affect the corticosterone level in response to stress, we examined CORT levels in the plasma of mice receiving the 2C5 mAb. As expected, the injection of 2C5 reduced corticosterone production in the plasma of WT mice subjected to CRS (FIG. 24B). This result, together with the observation that OPN promotes corticosterone production in OPN^{-/-} mice subjected to CRS, further reinforce the role of OPN in controlling corticosterone production.

TABLE 4
CRS-induced Reduction of Lymphocyte Populations.

| | WT | | | KO | | |
|---|---|---|---|---|---|---|
| | Blood | Thymus | Spleen | Blood | Thymus | Spleen |
| CD4+ | 22.3 ± 4.8 | ns | ns | ns | ns | ns |
| CD8+ | ns | ns | ns | ns | ns | ns |
| CD4+/ | na | 67.1 ± 9.5 | Na | na | 37.6 ± | na |
| CD8+ | | | | | 3.3 | |
| B220+ | ns | na | Ns | 33.1 ± 7.5 | na | ns |

Immune cells harvested from blood, thymus and spleen were stained with antibodies for CD4, CDB and B220 conjugated with fluoro-cytochromes. Percentages of each cell population were quantified by flow cytometry. Data for thymus and spleen were normalized to the organ weight and blood data were un-normalized. Data represent percent reduction of the respective populations in CRS-treated mice compared to unstressed controls.
Data with statistical significance at p < 0.05 were shown by mean ± SEM (n = 4 − 5).
Otherwise, ns = no significant difference; na = not applicable.

### Discussion

OPN is up regulated in various pathological and stress situations (18 Zohar et al., 2004; 4 Wang and Denhardt, 2008). Our previous research has revealed that OPN is critical for HU-induced lymphoid organ atrophy through modulating corticosteroid production. However, the role of OPN in other physical and psychological stress responses has remained un-explored. Both HU and CRS are physical stress models containing a significant psychological component that activates neural transmitters and stress hormones (19 Aviles et al., 2005; 20 Dhabhar et al., 2000). We have demonstrated here that OPN^{-/-} mice are resistant to CRS-induced lymphoid (largely thymus) organ atrophy. The stress-induced up-regulation of corticosterone production was significantly reduced in OPN^{-/-} mice. Thymus atrophy was easily detectable in CRS-treated mice whereas spleen weight loss was sometimes insignificant possibly because the stress level had not reached the threshold required to cause spleen atrophy. In the more stressful HU model, both spleen and thymus atrophy were consistently observed.

It is well documented that mice subjected to chronic physical stress exhibit an increased rate of lymphocyte apoptosis, redistribution of immune cells to the periphery and atrophy in the thymus and the spleen (11 Yin et al., 2000; 21 Offner et al., 2006). Based on these observations, lymphoid organ atrophy is a convenient marker for monitoring stress-induced changes in the immune system. The thymus is a primary lymphoid organ that consists of immature T cells and provides the environment for T cell development. It manifests dynamic physiological changes and is exquisitely sensitive to stress and toxic insult. It quickly responds to chemical and physical challenges, consequently leading to loss of cortical lymphocytes by apoptosis followed by organ atrophy (22 Pearse, 2006). Our data demonstrate both that injection of OPN into OPN-deficient mice enhances the CRS-induced thymus atrophy and that injection of a specific anti-OPN monoclonal antibody (2C5) into wild type mice ameliorates stress-induced thymus atrophy; changes in corticosterone levels were also partially reversed. This study reveals that OPN is one of the factors contributing to stress-induced organ atrophy and that it plays a significant role in immune cell survival/redistribution following chronic physical stress.

Our research has demonstrated that OPN is necessary for stress-induced corticosteroid up-regulation, possibly by affecting the production or metabolism of corticosteroid. We hypothesize this on the basis of the elevated basal level of corticosterone in OPN^{-/-} mice. Lack of OPN leads to an unchecked accumulation of corticosterone without stress stimulation, which could result from either increased production or decreased catabolism of corticosterone. We also report that ACTH levels were reduced in OPN^{-/-} mice and did not respond to CRS, implying either that a high basal level of corticosterone in OPN^{-/-} mice may repress ACTH release through the negative feedback regulation of CORT or that OPN has a direct effect on ACTH production. These findings suggest that circulating OPN mediates stress responses in the immune system possibly through regulating HPA hormone levels revealing that OPN is an important link between the immune system and the endocrine system.

Stress sends signals to the brain to induce the cascading release of the stress hormones ACTH, and CORT. The HPA axis is a major part of the neuroendocrine system that controls reactions to stress and regulates various physiological processes including immune responses. The level of these hormones in the plasma changes in response to stress and each of them has been shown to interact closely with the immune system in a bi-directional manner (23 Chen et al., 2004). Production of cytokines can stimulate the release of glucocorticoids; in turn, HPA activation by cytokines has been found to play a critical role in restraining and shaping immune responses. Thus, cytokine-HPA interactions represent a fundamental mechanism of the maintenance of homeostasis and the development of disease during stress or infection (24 Calcagni and Elenkov, 2006). For example, HPA axis hormones play an important role in autoimmune diseases such as multiple sclerosis (25 Heesen et al, 2007), adjuvant-induce arthritis, eosinophilia myalgia syndrome, systemic lupus erythematosus (26 Harbuz et al., 1997). Additionally, since OPN expression is highly perturbed in cancer, autoimmune and inflammatory diseases, manipulating OPN levels may have a wide impact on the overall health. By determining the role of lymphocyte death in a stressed immune system and the factors contributing to this process, we may be able to develop therapies to maintain a healthy immune system, which can help prevent malignancy and infections and treat stress-related illnesses.

### REFERENCES FOR EXAMPLE IV

1. Denhardt, D. T., M. Noda, A. W. O'Regan, D. Pavlin, and J. S. Berman. 2001. Osteopontin as a means to cope with environmental insults: regulation of inflammation, tissue remodeling, and cell survival. J Clin Invest. 107:1055-1061.
2. Sodek, J., A. P. Batista Da Silva, and R. Zohar. 2006. Osteopontin and mucosal protection. J Dent Res. 85:404-415.
3. Scatena, M., L. Liaw, and C. M. Giachelli. 2007. Osteopontin: a multifunctional molecule regulating chronic inflammation and vascular disease. Arterioscler Thromb Vasc Biol. 27:2302-2309.
4. Wang, K. X. and D. T. Denhardt. 2008. Osteopontin: Role in immune regulation and stress responses. Cytokine Growth Factor Reviews (in press).
5. Itoh, Y., T. Yasui, A. Okada, K. Tozawa, Y. Hayashi, and K. Kohri. 2005. Examination of the anti-oxidative effect in renal tubular cells and apoptosis by oxidative stress. Urol Res. 33:261-266.
6. Fujihara, S., M. Yokozeki, Y. Oba, Y. Higashibata, S, Nomura, and K. Moriyama. 2006. Function and regulation of osteopontin in response to mechanical stress. J Bone Miner Res. 21:956-964.
7. Wai, P. Y., and P. C. Kuo. 2004. The role of Osteopontin in tumor metastasis. J Surg Res. 121:228-241.
8. Wang, K. X., Shi, Y., and D. T. Denhardt. 2007. Osteopontin regulates hindlimb-unloading-induced lymphoid organ atrophy and weight loss by modulating corticosteroid production. Proc Natl Acad Sci USA. 104:14777-14782.
9. Sonnenfeld, G. 2005. The immune system in space, including Earth-based benefits of space-based research. Curr. Phar. Biotech. 6:343-349.
10. Nacher, J., K. Pham, V. Gil-Fernandez, and B. S. McEwen. 2004. Chronic restraint stress and chronic corticosterone treatment modulate differentially the expression of molecules related to structural plasticity in the adult rat piriform cortex. Neuroscience. 126:503-509.
11. Yin, D., D. Tuthill, R. A. Mufson, and Y. Shi. 2000. Chronic restraint stress promotes lymphocyte apoptosis by modulating CD95 expression. J Exp Med 191:1423-1428.
12. Zhang, Y., M. Woodruff, Y. Zhang, J. Miao, G. Hanley, C. Stuart, X. Zeng, S. Prabhakar, J. Moorman, B. Zhao, and D. Yin. 2008. Toll-like receptor 4 mediates chronic restraint stress-induced immune suppression. J Neuroimmunol. 194:115-122.
13. Bowers, S. L., S. D. Bilbo, F. S. Dhabhar, and R. J. Nelson. 2008. Stressor-specific alterations in corticosterone and immune responses in mice. Brain Behav Immun. 22:105-113.
14. Wang, K. X. 2008. Osteopontin: Role in Immune Regulation and Stress Responses. Doctoral Dissertation. Rutgers, The State University of New Jersey.
15. Keller-Wood, M. E., and M. F. Dallman. 1984. Corticosteroid inhibition of ACTH secretion. Endocr Rev. 5:1-24.
16. Kowalski, A. J. 2005. Creation, Characterization, and Application of Novel Anti-Osteopontin Monoclonal Antibodies. Doctoral Dissertation. Rutgers, The State University of New Jersey.
17. Kazanecki, C. C., A. J. Kowalski, T. Ding, S. R. Rittling, and D. T. Denhardt. 2007. Characterization of anti-osteopontin monoclonal antibodies: Binding sensitivity to post-translational modifications. J Cell Biochem. 102:925-935.
18. Zohar, R., B. Zhu, P. Liu, J. Sodek, and C. A. McCulloch. 2004. Increased cell death in osteopontin-deficient cardiac fibroblasts occurs by a caspase-3-independent pathway. Am J Physiol Heart Circ Physiol. 287:H1730-1739.
19. Aviles, H., T. Belay, M. Vance, and G. Sonnenfeld. 2005. Effects of space flight conditions on the function of the immune system and catecholamine production simulated in a rodent model of hindlimb unloading. Neuroimmunomodulation 12:173-181.
20. Dhabhar, F. S., A. R. Satoskar, H. Bluethmann, J. R. David, and B. S. McEwen 2000. Stress-induced enhancement of skin immune function: A role for Y interferon. Proc Natl Acad Sci USA. 97:2846-2851.
21. Offner, H., S. Subramanian, S. M. Parker, C. Wang, M. E. Afentoulis, A. Lewis, A. A. Vandenbark, and P. D. Hurn 2006. Splenic atrophy in experimental stroke is accompanied by increased regulatory T cells and circulating macrophages. J Immunol 176:6523-6531.
22. Pearse, G. 2006. Histopathology of the thymus. Toxicol Pathol. 34:515-547.
23. Chen, C. C., and C. R. Parker Jr. 2004. Adrenal androgens and the immune system. Semin Reprod Med. 22:369-377
24. Calcagni, E., and I. Elenkov. 2006. Stress system activity, innate and T helper cytokines, and susceptibility to immune-related diseases. Ann N Y Acad. Sci. 1069:62-76.
25. Heesen, C., S. M. Gold, I. Huiting a, and J. M. Reul J M. 2007. Stress and hypothalamic-pituitary-adrenal axis function in experimental autoimmune encephalomyelitis and multiple sclerosis. Psychoneuroendocrinology. 32:604-618.
26. Harbuz, M. S., G. L. Conde, O. Marti, S. L. Lightman, and D. S. Jessop. 1997. The hypothalamic-pituitary-adrenal axis in autoimmunity. Ann N Y Acad. Sci. 823:214-224.
27. Rittling, S. R., H. N. Matsumoto, M. D. McKee, A. Nanci, X. R. An, K. E. Novick, A. J. Kowalski, M. Noda, and D. T. Denhardt. 1998. Mice lacking osteopontin show normal development and bone structure but display altered osteoclast formation in vitro. J Bone Miner Res. 13:1101-1111.
28. Natasha, T., M. Kuhn, O. Kelly, and S. R. Rittling. 2006. Override of the osteoclast defect in osteopontin-deficient mice by metastatic tumor growth in the bone. Am J. Pathol. 168:551-561.
29. Shinohara, M. L., L. Lu, J. Bu, M. B. Werneck, K. S. Kobayashi, L. H. Glimcher, and H. Cantor. 2006. Osteopontin expression is essential for interferon-alpha production by plasmacytoid dendritic cells. Nat. Immunol. 7:498-506.
30. Wu, Y., D. T. Denhardt, S. R. Rittling. 2000 Osteopontin is required for full expression of the transformed phenotype by the ras oncogene. Br J Cancer. 83:156-163.
31. Christensen, B., M. S, Nielsen, K. F. Haselmann, T. E. Petersen, and E. S. Sørensen. 2005. Post-translationally modified residues of native human osteopontin are located in clusters: Identification of 36 phosphorylation and five O-glycosylation sites and their biological implications. Biochem J. 390:285-292.

### Example V

### Osteopontin in Autoimmune Demyelination

The following materials and methods are provided to facilitate the practice of this example.

We will induce EAE as previously described with either PLP 139-151 or MOG 35-55, in SJL or C57B1/6 respectively (Chabas et al, 2001; Hur et al, 2007; Ousman et al, 2007; Han et al, 2008). At the onset of disease, we will administer daily doses of 200 micrograms of anti-OPN monoclonals. We initially test each of the monoclonals that are cross-reactive with both mouse and human OPN. We will test them in both the relapsing remitting model of EAE in the SJL mouse with PLP 139-151 and in the progressive model of EAE in the C57B1/6. Outcome measures will include daily mean clinical score analyzed via Mann Whitney and Linear Regression, as well as monitoring of relapse frequency and mortality. At the end of experiments on day 60, we will remove brain and spinal cord and assess histology including counts of perivascular cuffs as we have routinely done in Chabas et al, 2001; Hur et al, 2007; Ousman et al, 2007; Han et al, 2008). For induction of EAE, monitoring of EAE, scoring of pathology: See publications Chabas et al, 2001; Hur et al, 2007; Ousman et al, 2007; Han et al, 2008.

We have shown that anti CD44 and anti-alpha 4 integrin block EAE (Brocke et al, 1999). Yednock et al, 1992). Interleukin [IL-12] production is modulated through an integrin/osteopontin interaction, while IL-10 is modulated via CD44 (Ashkar et al, 2000). We have now seen that antibodies to osteopontin modulate not only TH1 but TH17. We will characterize the hierarchy of cytokine production in EAE and MS between OPN, TH1 and TH17. We will determine whether the effect on TH17 is mediated via T cells, antigen presenting cells or both cell types. Osteopontin is both pro-inflammatory and pro-survival on the immune system, and these two effects certainly synergize to account for disease progression and relapses. The effects on the pro-inflammatory TH1 and TH17 pathways as well as the effects of osteopontin on chemotaxis will be determined.

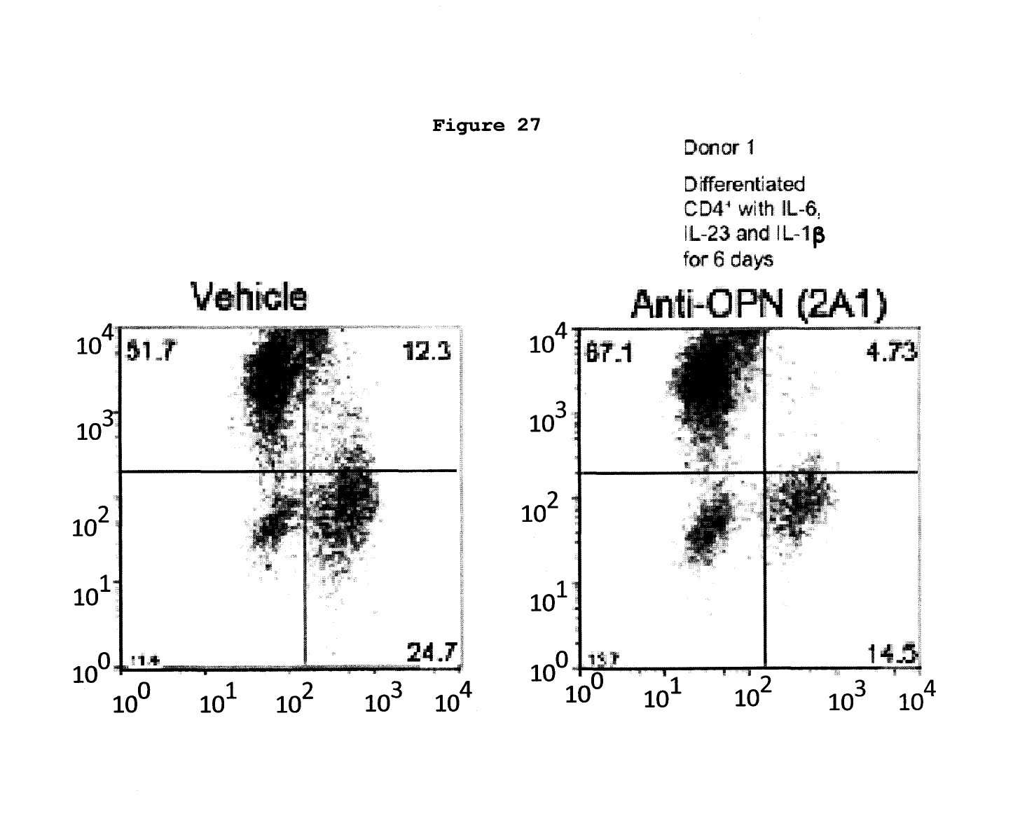

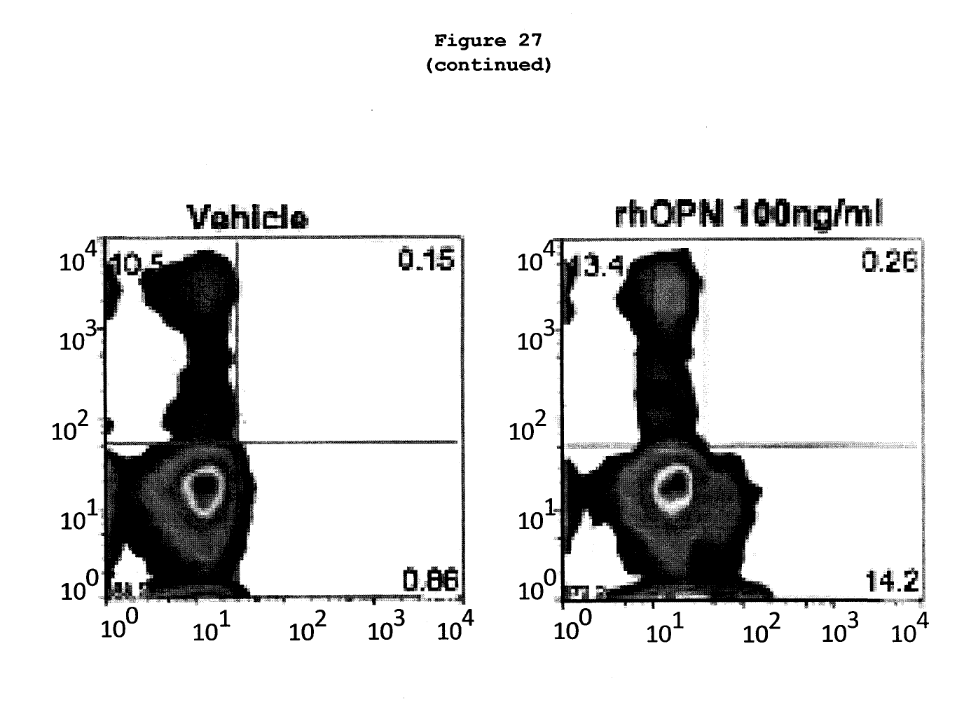

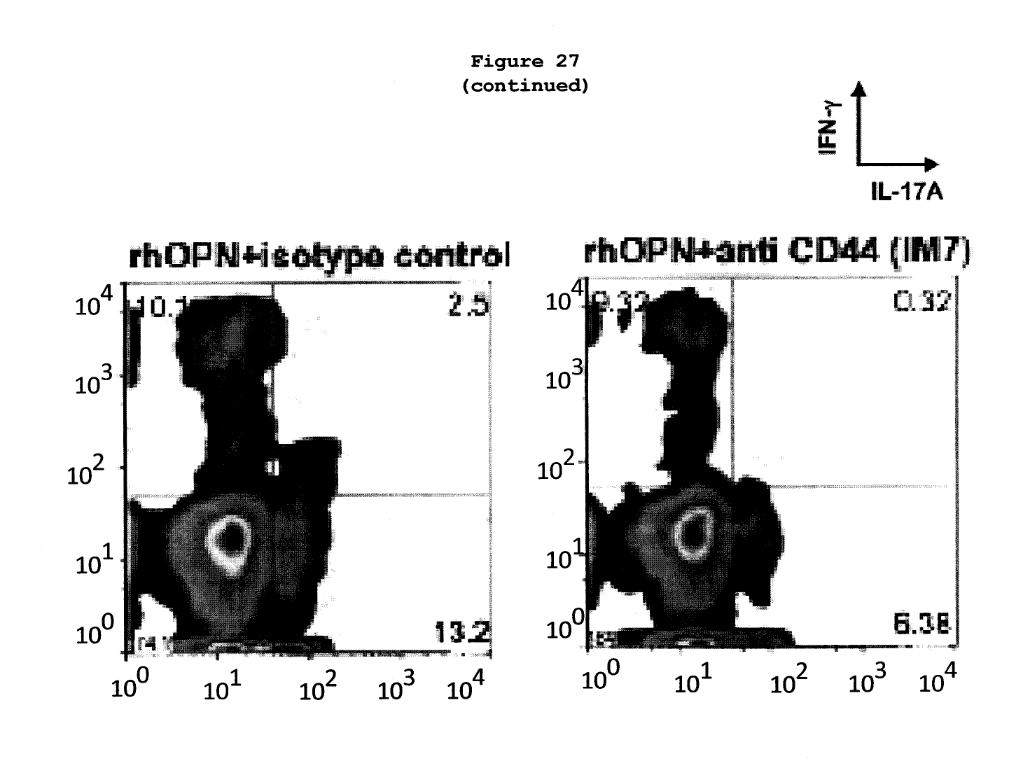

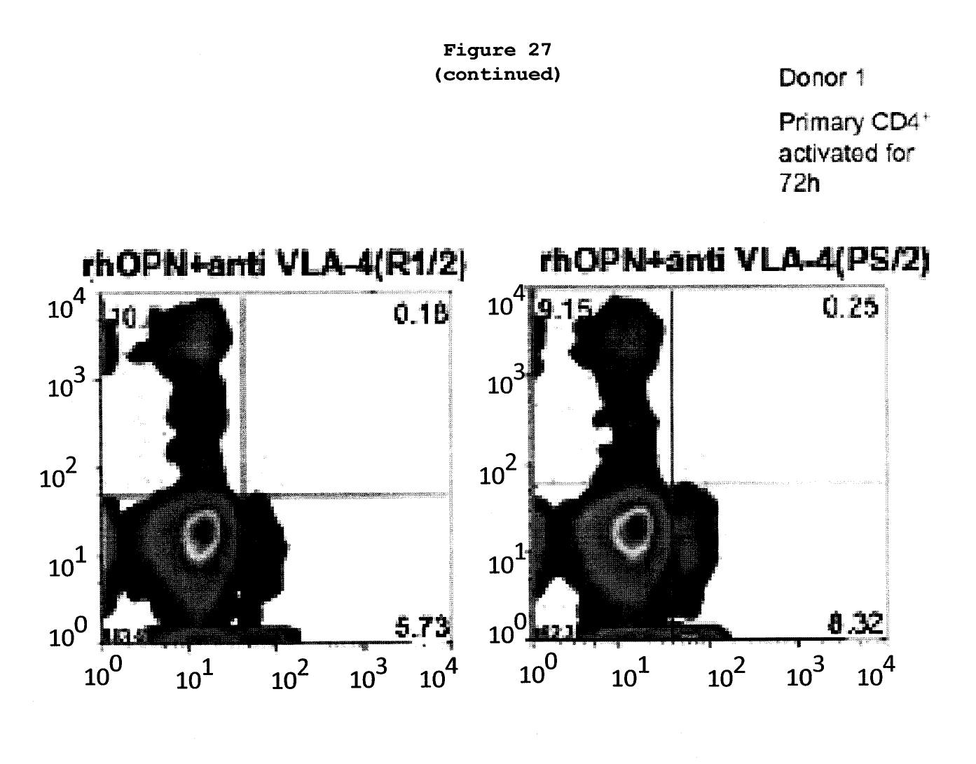

We will test 20 healthy donors, 10 male and 10 female, with the following protocol for assessing OPN modulation of TH1 and TH17 as well as OPN modulation of chemotaxis. Preliminary data are shown in FIGS. 27A and 27B showing that OPN drives both TH1 and TH17 cytokines and that anti-a4b1 antibodies and antiCD44 antibodies can inhibit this expansion. We will strive to test patients who are in three categories: Off all beta interferons and glatiramer, those on beta interferons and not on glatiramer, and those on glatiramer but not interferons.

Protocol for assessing the capacity of osteopontin to modulate TH1 and TH17 and for this modulation to be blocked with anti-OPN mabs:

80 ml of peripheral blood from healthy donors is obtained. Blood is collected into heparin coated standard blood collection tubes and diluted 1:1 with PBS containing 2% FBS, layered over Ficoll, and centrifuged for 20 minutes at 1200×g to obtain PBMCs. Post centrifugation, the enriched PBMCs were collected from the plasma-Ficoll interface, washed to remove excess of Ficoll. CD4⁺ cells were enriched from these samples using MACS magnetic Human CD4 Microbeads (Miltenyi Biotec, according to the manufacturer's instruction). T cells are isolated at 93-97% purity as confirmed by fluorescence-activated cell sorting (FACS) using BD LSR (BD Biosciences). Cells are cultured in 96-well round-bottomed plates (Falcon) at 1×10⁶ cells/ml in serum free media X-Vivo 15 (Lonza), supplemented with 100 units/ml penicillin/streptomycin (Invitrogen), 14.3 μM β-mercaptoethanol (Sigma-Aldrich), and L-glutamine (Invitrogen). The T cells are activated with Dynabeads CD3/CD28 T Cell Expander (Invitrogen) at 1×10⁶ beads/ml.

As we show in FIG. 27A we induce expansion of IL-17 secreting cells CD4⁺ T cells by culturing with 10 ng/ml of human recombinant IL-1□, IL-6 and 5 ng/ml of IL-23, (all from eBioscience). 48 h later cells were transferred to fresh media for expansion with the additional fresh beads and cytokines and incubated for another 48 h. At day 4 cells were washed and plated at a concentration of 1×10⁶/ml with fresh media with or without 10 μg/ml of anti-OPN monoclonal antibody (clone 2A1, or 3D9 or isotype control). Beads were then added in concentration of 10⁴/ml to continue expansion of the cells. 48 h hours after antibody incubation, live cells were assayed for IL-17 and IFN-γ secretion via flow cytometry as described below. FIG. 27a shows IL-17 and IFN-γ secretion of CD4 treated cells from donor 1 treated with 2A1a monoclonal antibody that also diminishes EAE when given after the onset of paralysis, versus isotype control. Clone 3D9 showed similar pattern of results but was weaker in inhibiting IL-17 secretion. All donors expressed similar patterns of secretion data not shown for limited space.

In FIG. 27B we tested whether OPN can induce IL-17 expansion. Same CD4+ T cells were incubated with dynabeads as described above with the addition of 100 ng/ml of human recombinant OPN(R&D Systems). To test the direct effect of OPN on secretion IL-17 and IFN-γ cells were pre-incubated for 4 hours prior the addition of OPN and the beads with antibodies to several receptors for OPN i.e.: anti CD44 monoclonal antibody (1 μg/ml, clone IM7, BD Biosciences) or anti VLA-4 monoclonal antibody (clones 10 μg/ml R1/2 or 5 μg/ml PS/2, isolated from ascitcs in-house), or isotype control. All clones were verified to cross react with human determinants and optimal concentrations were tested prior to the experiment. After 4 h of pre-incubation with the antibodies, OPN and the dynabeads were added to the CD4⁺ T cells and were incubated for 72 h. IL-17 and IFN-γ secretion were assayed via flow cytometry analysis as described below.

To measure cytokine secretion post treatment cells are washed and plated with fresh serum free media and stimulated for 5 h with PMA (30 ng/ml, Sigma) and ionomycin (750 nM, sigma), and monensin (GolgiStop™, BD Biosciences according to the manufacturers protocol). Cell are stained with APC-Cy7-conjugated anti human CD4 antibody (BD Biosciences, 557871). Next, for intracellular cytokine secretion, cells are fixed with 4% PFA and permeabilized using BD Cytofix/Cytoperm kit (BD Biosciences, 554715). After pemeabilization cells are stained with PE-conjugated anti-Human IL-17 (eBiosciences, #12-7179-73) and APC-conjugated anti-human IFNγ (BD Biosciences, #554702) antibody. All data are stored on the BD LSR system (BD Biosciences) and data were analyzed using FlowJo software.

The human chemotaxis assay, is performed as follows: T-cell migration will be measured using transwell inserts (membrane pore size 5 μm, Becton Dickinson and Co, Franklin Lakes, N.J., USA) pre-equilibrated in culture medium (RPMI-1640) overnight at 4° C. Lymphocytes from 20 human volunteers [10 males and 10 females] will be seeded individually in transwells at 1×10⁶ cells per 100u1 per well. The bottom wells are loaded with 600 μl of assay medium or 600 μl of OPN at various concentrations. The T cell chemoattractant MIP-3P will be as used as a positive control. Blockade of migration will be tested with various anti-OPN mabs at various concentrations. Transwells will be immersed in chemoattractant containing media and were incubated in 37° C., 5% CO₂ incubator for 3 hours. Cells migrated through the membrane to the bottom wells were collected and counted with FACS Calibur (Becton Dickinson) for 30 sec.

### Preventive Regimen for EAE:

Active EAE will be induced in C57BL/6 mice using a standard protocol in which maB's to opn that are effective in blocking EAE like 2A1, or PBS, will be injected every two days, 200 micrograms, beginning on the day of disease induction until the experiment is terminated. The severity of EAE in the mice will be scored as follows: 0=no clinical disease, 1=limp tail, 2=hind limb weakness, 3=complete hind limb paralysis, 4=hind limb paralysis plus some forelimb paralysis, and 5=moribund or dead. To gain insight into the cell types that are affected by anti-OPN treatment, lymph nodes [LN], brain and spinal cords will be collected at three specific time points during EAE progression and analyzed for changes in dendritic cells, macrophages and CD4+ T-cells.

The first time point will be at day 3-day 6 post EAE induction. This is the time period dendritic cells drain to the lymph nodes and present antigen to T-cells following immunization. The absolute numbers and activation state of dendritic cell subsets (lymphoid, myeloid and plasmacytoid) will be characterized by flow cytometry. To determine if anti-OPN treatment alters the function of dendritic cells, DC's will we be isolated by magnetic separation and cultured to assess their capacity to process and present whole MOG protein to activate and differentiate naïve MOG specific CD4 T-cells, isolated from 2D2 MOG TCR transgenic mice, into effector TH cells.

The second time point will be day 9-11 post EAE induction. This is the period just prior to the onset EAE when effector CD4 T-cells have been fully differentiated in the lymph nodes and are also beginning to be detected in the spinal cord. Dendritic cells are important for the differentiation of T helper cells in to Th17, Th1 and regulatory T-cells [T-reg]; however, the effect that anti-OPN treatment has on this process has not been fully elucidated. Therefore, T-cells and DCs from LN and spinal cord will be evaluated for production of Th17-associated cytokines [TGF-β, IL-6, IL-23, IL-21, IL-17, TNF-α], Th1-associated cytokines [IFNγ, IL-12, IL-18, IFN-α/β] and T-reg cytokines [IL-10 and TGF-β] by intracellular flow cytometry and ELISA. In addition, Th2 cytokines and [IL-4, IL-5] will be assessed.

The third time point will be at the peak of EAE symptoms between day 17-21 post induction of disease. This is the period where, CD4 T-cells produce large quantities of inflammatory cytokines and macrophages and other myeloid dendritic cells have infiltrated into the CNS and subsets of these populations will be assessed by flow cytometry and immuno-histochemistry.

### Treatment Regimen:

Injection with anti-OPN 200 micrograms or PBS will begin when mice exhibit a clinical score of 2 to 3 and continue every day until the termination of the experiment. We have shown that this regimen begins to show efficacy after four doses and has a maximal effect after about 10 days. Therefore, we will sacrifice mice 2 days after the 4^{th} dose of treatment and LN spleens and spinal cords will be analyzed for differences in the phenotype and function T-cell and DC's cells as described by flow cytometry, immunohistochemistry and cell culturing as described above.

### Results

400,000 Americans suffer from multiple sclerosis [MS]. Both MS, and its animal model, experimental autoimmune encephalomyelitis [EAE], have both progressive and relapsing/remitting forms (Steinman and Zamvil, 2003; Steinman 2003). The factors underlying the transformation of autoimmune demyelinating disease from a relapsing and remitting form to the more devastating progressive aspect remain to be elucidated. In 2001, we discovered that osteopontin, OPN, is a critical gene encoding a protein expressed in MS lesions (Chabas et al, 2001), and that it may play a role in the transformation from relapsing remitting disease to the more chronic form. (Chabas et al, 2001).

OPN serves as a ligand for two adhesion molecules, CD44 and alpha 4 integrin (Chabas et al, 2001; Brocke et al, 1999) Anti-alpha4 integrin, Natalizumab, a drug approved for treatment of MS, has now been shown to reduce relapses by 66% over two years in patients with MS, and is now approved by the FDA and available under the name Tysabri. Though Natalizumab lead to 3 cases, two fatal, of PML in the first 3000 patients who were treated, under new guidelines the treatment has been given to 30,000 patients with no further relapses since its reintroduction in 2008.

In light of these data, it appears that blockade of osteopontin provides a new therapeutic for treatment of MS.

Recent data has shown that blockade of OPN with monoclonal antibodies reduces both TH1 and TH17 responses in EAE and in human T cells. Thus a monoclonal antibody described herein has shown promise in reducing relapsing EAE. Further work shows that OPN is capable of inducing relapses in EAE, via inhibition of apoptosis [Hur et ai, 2007J. It appears that OPN induces relapses by inhibiting the phosphorylation of Fox03a. Administration of antibodies via intravenous infusion is known to the skilled artisan and is the preferred route of delivery for the anti-OPN antibodies described herein. Dosing can be ascertained by the clinician and depends on the severity of the disease to be treated and the weight and condition of the patient.

Progressive paralysis ensues in mice after injection of myelin oligodendroglial glycoprotein [MOG] peptide 35-55 in complete Freund's adjuvant. In OPN−/− knock out mice, maintained on a 129/C57BL/6 outbred strain (Rittling et al, 1998) and injected with MOG 35-55, progressive EAE is rare, while remissions of disease are common. EAE was observed in 100% of both OPN+/+ and OPN−/− mice with MOG 35-55. Despite this, severity of disease was reduced in all animals in the OPN−/− group, and these mice were totally protected from EAE-related death (Chabas et al, 2001). During the first 26 days, OPN−/− mice displayed a distinct evolution of EAE, with a much higher percentage of mice having remissions compared to the controls.

Differences in cytokine expression in these mice confirmed that OPN was pivotal in controlling Th1/Th2 polarization. T cells in OPN−/− mice showed a reduced proliferative response to MOG 35-55, compared with OPN+/+ T cells. In addition, IL-10 production was increased in T cells reactive to MOG 35-55 in OPN−/− mice that had developed EAE, compared with T cells in OPN+/+ mice. At the same time, IFN-gamma [IFN-γ] and IL-12 production were diminished in cultures of spleen cells stimulated with MOG. Earlier work by Cantor's group had shown that IL-12 is modulated via an OPN interaction with integrins, and that IL-10 is modulated via an interaction of OPN with CD44 (Ashkar et al, 2000).

Both beta 3 integrin and alpha 4 integrin may interact with OPN (Ashkar et al, 2000; Pepinsky et al, 2002), though modulation of IL-12 has been shown so far to work best via beta 3, “Induction of IL-12 is inhibited by GRGDS peptide (but not GRADS peptide) and by antibody to the integrin 3 subunit (Ashkar et ai, 2000).” Monoclonals like 2C5, which bind near to the RGD sequence of OPN, appear to modulate the OPN-integrin interaction thereby influencing cytokine modulation. Recent work contained in the progress report show that osteopontin modulates both TH1 and TH17 cytokines.

High throughput sequencing of cRNA from expressed sequence tags [EST], utilizing non-normalized cDNA brain libraries generated from MS brain lesions and control brain, has revealed the most prominent transcripts found in MS brain (Chabas et al, 2001). We sequenced over 11,000 clones from these libraries from MS patients and controls, respectively, and concentrated the analysis on genes present in both MS libraries, but absent in the control library. This yielded 423 genes, including 26 novel genes. From those, 54 genes showed a mean-fold change of 2.5 or higher in libraries derived from MS brain. Transcripts for alpha B-crystallin, an inducible heat shock protein, localized in the myelin sheath, and targeted by T cells in MS, were the most abundant mRNAs to be unique to MS plaques. The next five most abundant transcripts, included those for prostaglandin D synthase, prostatic binding protein, ribosomal protein L 17, and OPN.

Given the known inflammatory role for OPN, we examined the cellular expression pattern of this protein in human MS plaques and in control tissue, by immunohistochemistry. Within active MS plaques, OPN was found on microvascular endothelial cells and macrophages, and in white matter adjacent to plaques. Reactive astrocytes and microglia also expressed OPN (Chabas et al, 2001; Zamvil and Steinman, 2003). Esiri and Sinclair have shown osteopontin on inflamed endothelium in MS brain, as well as axons and astrocyte endfeet [Diaz Sanchez et al, 2006; Sinclair et al, 2005].

Additional studies examining OPN polymorphisms and disease course in MS, also implicate a role for osteopontin in MS. In 821 MS patients, a trend for association with disease course was detected in patients carrying at least one 1284A allele in the OPN gene. Patients with this genotype were less likely to have a mild disease course and were at increased risk for a secondary-progressive clinical type (Caillier et al, 2003).

Elevated levels of OPN have been seen in plasma during relapses of MS (Vogt et al, 2003), and these OPN elevations occur up to a month earlier than the appearance of new Gadolinium enhancing lesions (Vogt et al, 2004). These studies were again confirmed: Patients with RRMS during relapse presented higher OPN levels than patients with RRMS during clinical remission. (Comabella et al, 2005).

Recent work from our lab shows that osteopontin is a pro-survival molecule inhibiting apoptosis of autoimmunogenic T cells [Hur et ai, 2007]. OPN works via modulation of the phosphorylation of Fox03a and NF-kB.

Osteopontin has recently been shown not only to increase TH1 cytokines as shown in Chabas et al, 2001 and Jansson et al. 2002, but now is shown to up regulate TH17 cytokines. Shinohara and colleagues show that modulation of the Th17 axis in EAE occurs via type-1 interferon receptors expressed in dendritic and microglial cells. The type-1 interferon receptors inhibit intracellular osteopontin and thereby downregulate production of IL-17 in pathogenic T cells [Shinohara et al, Immunity 2008]. This work connects the role of beta interferon [Amason, 1999], an approved drug for treatment of relapsing MS, with osteopontin, a molecule that can induce relapses in several models of EAE [Hur et al, 2007], and whose elevation in plasma is associated with relapses in MS [Vogt et al 2003]. Both Th1 and Th17 are involved in relapses in both EAE and in MS [Steinman, 2008; Bettelli et al, 2008].

Relapses in MS—A Duet between Natalizumab and Osteopontin

In 1992, we discovered that α4β1 integrin was the critical adhesion molecule in homing to the inflamed brain [Yednock et al, 1992; Steinman, 2005]. Osteopontin is found on inflamed endothelium [Diaz Sanchez et al, 2006; Sinclair et al, 2005]. Remarkably osteopontin is a member of the SIBLING family of proteins and binds several integrins including αvβ3 and α4β1 integrin [Ashkar et al, 2000; Pepinsky et al, 2002]. As noted osteopontin itself is associated with relapse. This duet between α4β1 integrin and osteopontin is critically entwined with relapse in MS: Blockade of α4β1 reduces relapses in MS by two thirds, while osteopontin is elevated in plasma around the time of relapses in MS, and its administration to mice with EAE, quickly triggers neurological relapse via two mechanisms. First osteopontin elevates pro-inflammatory mediators, including TH1 [Chabas et al, 2001] and TH17 cytokines [Shinohara et al, 2008] and second, osteopontin inhibits Fox03a dependent apoptosis of immune cells [Hur et al, 2007].

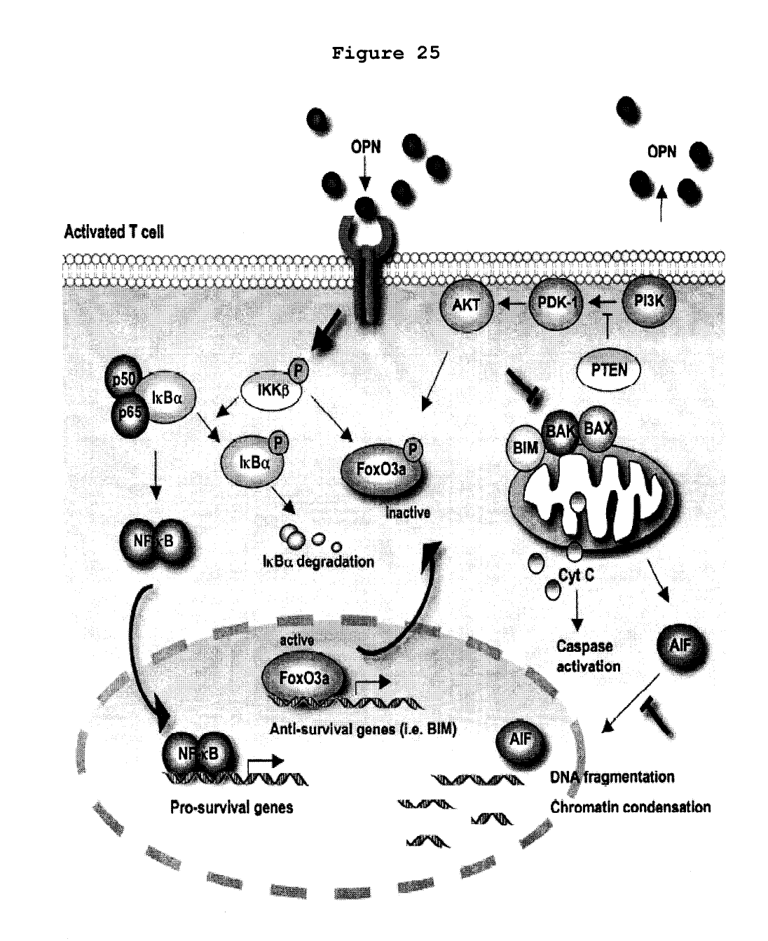

FIG. 25 depicts a diagram of OPN-induced survival of T cells. OPN induces phosphorylation and retention in cytosol of FoxO3a. NF-kB activation is also induced by OPN. The inhibition of FoxO3a along with activation of NF-kB results in induction of pro-survival proteins. The expression of anti-survival Bcl-2 family proteins, Bim, Bak and Bax is altered by OPN. Translocation of AIF to nucleus from mitochondria, where AIF plays role as a pro-survival protein, is inhibited by OPN [data appears in Hur et al, 2007].

Identified Opn-induced mechanisms that could influence disease progression include enhanced survival of activated T cells in the CNS (1); increased IL-12 production by macrophages (2); enhanced secretion of proinflammatory cytokine production by T cells; 3); increased migration of monocytes and T cells into the CNS (4), which could lead to increased determinant spreading (5); possible inhibition of IL-10 produced by regulatory T cells (6); possible inhibition of IL-10 produced by B cells (7); and activation of astrocytes [Stromnes and Goverman, 2007].

### Effects of OPN on IL-17 Production in Mouse and Man

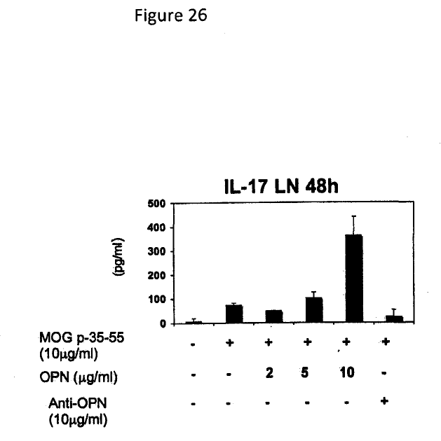

Work published in Science in 2001, Chabas et al, showed that OPN modulated TH1 cytokines. We now show that OPN modulates TH17 as well. See FIGS. 26 and 27.

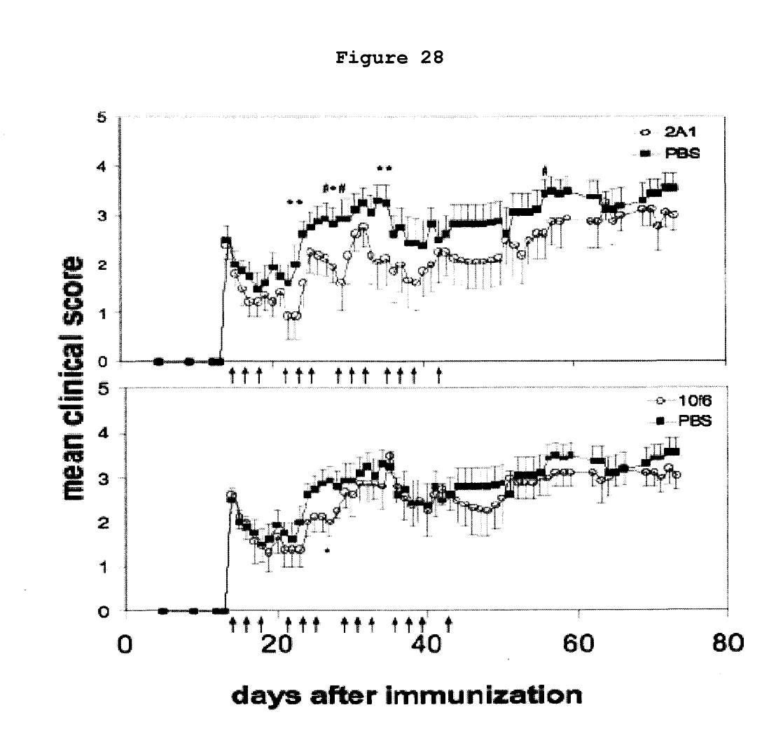

In earlier studies, it was shown that recombinant osteopontin is associated with relapses and exacerbation of EAE symptoms. In the following studies, we describe a blockade of disease progression as a result of administration of the osteopontin antibodies of the invention. As shown in FIG. 28, we have been able to attenuate EAE after the onset of disease with a monoclonal OPN antibody, 2A1, given 200 micrograms on the days indicated by arrows in the figure.

As mentioned above, more than 30 hybridomas producing antibodies to both mouse and humans have been developed in accordance with the present invention. These include monoclonals that bind well (elisas, westerns, peptides) to human native osteopontin (from human milk). About a third, so far, have been shown to react with specific peptides from OPN, thus localizing their cognate epitopes to different regions of the OPN molecule.

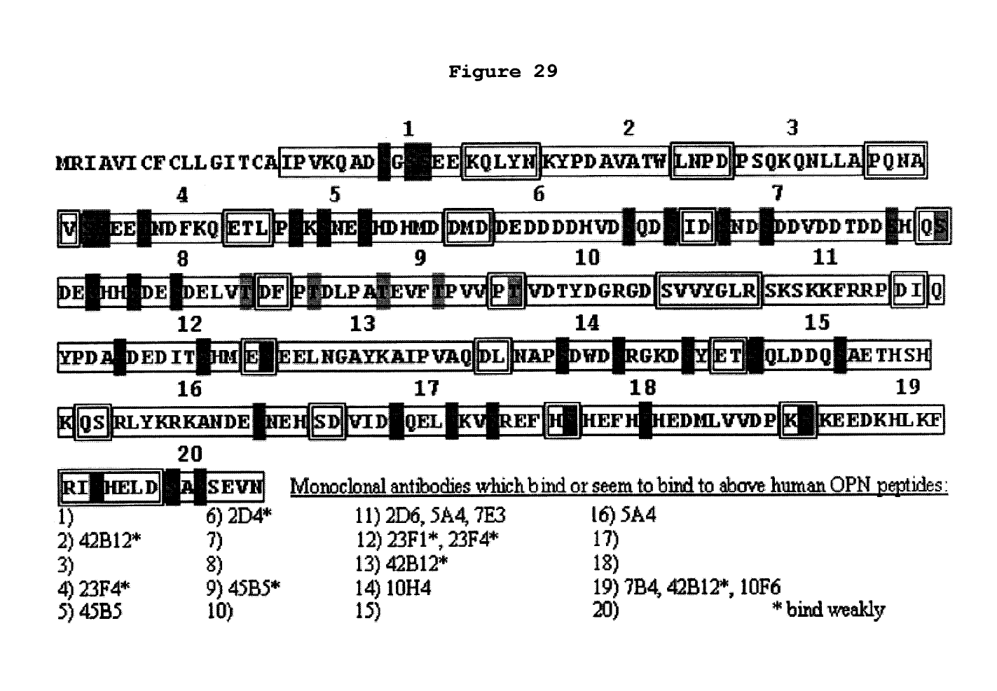

One or more anti-OPN monoclonal antibodies should exhibit efficacy in the treatment of autoimmune disease as discussed above. Inasmuch as OPN is known to promote the progression of autoimmune diseases (e.g. EAE, RA) in the mouse it is also playing a role in human disease. OPN exists in various isoforms with differing post-translational modifications. Differences in PTMs influence the functional behavior of OPN in both physiological and pathophysiological processes such as cell migration, cell adhesion, and cell proliferation. The ability to inactivate functionally specific isoforms of OPN with particular mAbs provides an essential step in elucidating OPN's in vivo actions. Using biotinylated peptides, the epitopes recognized by a subset of the monoclonals have been identified. See FIG. 29

### Example VI

### OPN Associated Modulation of Lymphocyte Chemotaxis

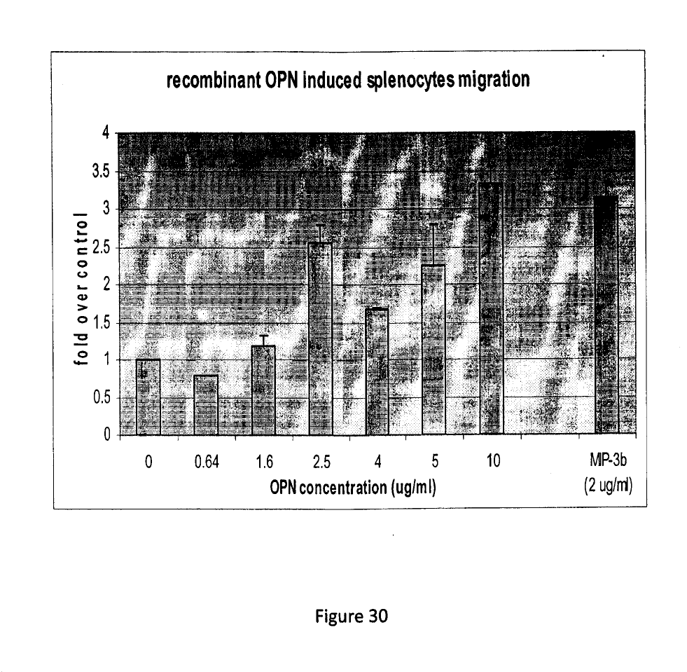

OPN mediates splenocytes chemotaxis through N-terminal half of the molecule. In order to determine the chemotaxis function of osteopontin, recombinant OPN was first used to establish a dose-dependent cell migration response. As shown in FIG. 30, recombinant OPN has been tested ranging from 0.64 μg/ml to 10 μg/ml. A steady increase in the number of cells migrating to the lower chamber correlated with the increase of the concentration of OPN. This result confirmed that full length recombinant OPN can induce splenocyte migration. Next, we used a number of thrombin digested and endoproteinase Lys-C digested bovine OPN fragments provided by Dr Sørensen to test their effect on inducing splenocytes chemotaxis. The fragments tested included the following:

### OPN Fragments

“SKK” (Thrombin OPN, AA's 148-204)

“AKDK” (Thrombin OPN, AA's 205-262)

“C18” (N-term OPN, AA's 1-145/147+4 O-gly)

“SP200” (N-term OPN, two N-term variants and a few minor contaminant of OPN origin)

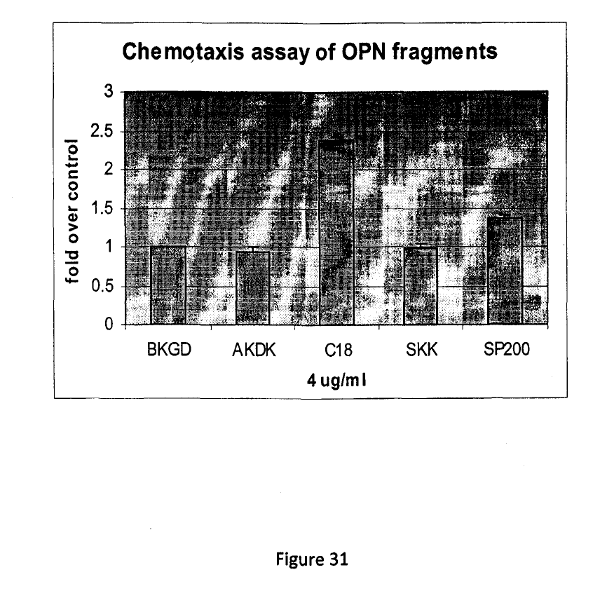

Data in FIG. 31 indicated that only one of the 4 fragments induced splenocyte migration, specifically highly purified N-terminal fragment with O-glycosylation. It is believed that OPN-mediated chemotaxis is largely dependent on its interaction with CD44 receptors. Therefore, this result implies that the chemotaxis function of OPN is conveyed by the N-terminal half of the molecule and a CD44 binding site is located in this region [Wang, 2008].

Lymphocyte Chemotaxis Assay with OPN

T-cell migration will be measured in the presence and absence of anti-OPNs using transwell inserts (membrane pore size 5 μm, Becton Dickinson and Co, Franklin Lakes, N.J., USA) pre-equilibrated in culture medium (RPMI-1640) overnight at 4° C. Cells will be seeded in transwells at 1×10⁶ cells per 100 ul per well. The bottom wells are loaded with 600 μl of assay medium or 600 μl of OPN at various concentrations. The T cell chemoattractant MIP-3β will be as used as a positive control. Transwells will be immersed in chemoattractant containing media and were incubated in 37° C., 5% CO₂ incubator for 3 hours. Cells migrating through the membrane to the bottom wells are collected and counted with FACS Calibur (Becton Dickinson) for 30 sec.

We will then assess the effects on chemotaxis of the various mAB's that cross-react with human and mouse osteopontin that have been used in the EAE assays, thereby determining the potency of mab's that prevent or reverse EAE in terms of their effects on TH1, TH17 and chemotaxis.

The lead candidates for testing in humans based on the mouse experiments are monoclonal antibodies recognizing human OPN, that 1) inhibits relapses in the SJL PLP139-151 model of relapsing EAE and 2) blocks progression in the C57B1/6 MOG 35-55 model of progressive EAE, that 3) diminishes both TH1 and TH17 cytokine production in EAE, and that 4) inhibits chemotaxis.

### REFERENCES FOR EXAMPLE VI

Arnason, B. G. W. 1999. Immunologic Therapy of Multiple Sclerosis. Ann Rev Med 50: 291-302
Ashkar S, Weber G F, Panoutsakopoulou V, Sanchirico M E, Jansson M, Zawaideh S, Rittling S R, Denhardt D T, Glimcher M J, Cantor H. Et al [osteopontin] an early component of type 1 immunity. Science 287:860-864, 2000
Bettelli, E., Oukka, M., Kuchroo, V. K. 2007. T(H)-17 cells in the circle of immunity and autoimmunity. Nat. Immunol. 8:345-350.
Brocke S, Piercy C, Steinman L, Weissman I L, Veromaa T. Antibodies to CD44 and integrin alpha 4, but not L-selectin, prevent CNS inflammation and experimental encephalomyelitis by blocking secondary leukocyte recruitment. Proceedings of the National Academy of Sciences USA, 96:6896-6901, 1999
Caillier S, Barcellos L, Baranzini S, Swerdlin A, Lincoln R, Steinman L, Oksenberg J R. Osteopontin Polymorphisms and Disease Course in MS, Genes and Immunity, 4:312-315, 2003.
Chabas D, Baranzini S, Mitchell D, Bernard C C A, Rittling S, Denhardt, D, Sobel R, Lock C, Karpuj M, Pedotti R, Heller R, Oksenberg J, Steinman L. The influence of the pro-inflammatory cytokine, osteopontin, on autoimmune demyelinating disease. Science, 294: 1731-1735, 2001.
Christensen, B, Kazanecki, C C, Petersen, T E, Rittling, S R, Denhardt, D T, and Sørensen, ES. (2007) Cell type-specific post-translational modifications of mouse osteopontin are associated with different adhesive properties. J Biol. Chem. 282:19463-72.
Comabella M, Pericot I, Goertsches R, Nos C, Castillo M, Blas Navarro J, Rio J, Montalban X. Plasma osteopontin levels in multiple sclerosis. J. Neuroimmunol. 2005 January; 158(1-2):231-9.
Craig A M, Smith J H, Denhardt D T. Osteopontin, a transformation-associated cell adhesion phosphoprotein, is induced by 12-O-tetradecanoylphorbol 13-acetate in mouse epidermis. Journal of Biological Chemistry, 264(16):9682-9. 1989.
Diaz-Sanchez M, Williams K, DeLuca G C, Esiri M M. Protein co-expression with axonal injury in multiple sclerosis plaques Acta Neuropathol. 2006 April; 111(4):289-999.
Garren H, Robinson W, Krasulová E, Havrdová E, Nadj C, Selmaj K, Losy J, Nadj I, Radue E W, Kidd B A, Gianettoni J, Tersini K, Utz P J, Valone F, Steinman L and the BHT-3009 Study Group. Phase 2b Trial of a DNA Vaccine Encoding Myelin Basic Protein in Relapsing Multiple Sclerosis. Annals of Neurology, 63(5):611-620, 2008
Han M H, Hwang S, Roy D B, Lundgren D H, Price J V, Ousman S, Fernald G, Gerlitz B, Robinson W H, Baranzini S E, Grinnell B W, Raine C S, Sobel R A, Han D K, and Steinman L. Proteomic Analysis of Active Multiple Sclerosis Lesions Reveals Therapeutic Targets. Nature, 451:1076-1081, 2008
Hur E, Youssef S, Haws M, Zhang S, Sobel, R, Steinman L. Osteopontin induced relapse and progression of autoimmune brain disease via enhanced survival of activated T cells. Nature Immunology, 8: 77-86, 2007
Jansson M, Panoutsakopoulou V, Baker J, Klein L, Cantor H. Cutting edge: Attenuated experimental autoimmune encephalomyelitis in eta-1/osteopontin-deficient mice. J. Immunol. March 1; 168(5):2096-9, 2002.
Kazanecki C C, Uzwiak D J, Denhardt D T. (2007) Control of osteopontin signaling and function by post-translational phosphorylation and protein folding. J Cell Biochem. 102:912-24.
Kazanecki C C, Kowalski A J, Ding T, Rittling S R, Denhardt D T. (2007) Characterization of anti-osteopontin monoclonal antibodies: Binding sensitivity to post-translational modifications. J Cell Biochem. 102:925-35.
Kowalski, A. J. 2005. Creation, Characterization, and Application of Novel Anti-Osteopontin Monoclonal Antibodies. Doctoral Dissertation. Rutgers, The State University of New Jersey
O'Regan A, Hayden J, Berman J. Osteopontin augments CD3-mediated interferon-gamma and CD40 ligand expression by T cells, which results in IL-12 production from peripheral blood mononuclear cells. J Leuk Bio 68:495-502, 2000.
Ousman S S, Tomooka B H, Van Noort J M, Wawrousek E F, O'Conner K, Hafler D A, Sobel R A, Robinson W H and Steinman L. Protective and Therapeutic Role for, aB-Crystallin in Autoimmune Demyelination, Nature 448:474-479, 2007
Patarca R, Freeman G J, Singh R P, Wei F Y, Durfee T, Blattner F, Regnier D C, Kozak C A, Mock B A, Morse HC 3rd, et al. Structural and functional studies of the early T lymphocyte activation 1 (Eta-1) gene. Definition of a novel T cell-dependent response associated with genetic resistance to bacterial infection. Journal of Experimental Medicine, 170(1):145-61, 1989
Pepinsky R B, Mumford R A, Chen L L, Leone D, Amo S E, Riper G V, Whitty A, Dolinski B, Lobb R R, Dean D C, Chang L L, Raab C E, Si Q, Hagmann W K, Lingham R B. Comparative assessment of the ligand and metal ion binding properties of integrins alpha9beta1 and alpha4beta1. Biochemistry 2002 Jun. 4; 41(22):7125-41
Remaley A, Schumacher U, Amouzaheh H, Brewer H and Hoeg J. Identification of novel differnetially expressed hepatic genes in cholesterol-fed rabbits by a non-targeted gene approach. Journal of Lipid Research 36:308-314, 1995.
Rittling S R, Matsumoto H N, McKee M D, Nanci A, An X R, Novick K E, Kowalski A J, Noda M, Denhardt D T. Mice lacking osteopontin show normal development and bone structure but display altered osteoclast formation in vitro. J Bone Miner Res. July;13(7):1101-11 (1998).
Rittling S and Denhardt D. Osteopontin function in pathology. Lessons from osteopontin deficient mice. Exp Neph 7: 103-113, 1999
Shinohara, M. L., Kim, J.-H., Garcia, V. A., and Cantor, H. 2008. Engagement of the Type-1 Interferon receptor on dendritic cells inhibits TH17 cell development. Central Role of Intracellular Osteopontin. Immunity, DOI10.1016/j.immuni2008.05.2008.
Sinclair C, Mirakhur M, Kirk J, Farrell M, McQuaid S.Up-regulation of osteopontin and alphaBeta-crystallin in the normal-appearing white matter of multiple sclerosis: an immunohistochemical study utilizing tissue microarrays. Neuropathol Appl Neurobiol. 2005 June; 31(3):292-303.
Sorensen S, Justesen S J, Johnsen A H. Purification and characterization of osteopontin from human milk. Protein Expr Purif. 2003 30:238-45.
Steinman L. Blocking Adhesion Molecules As Therapy For Multiple Sclerosis: Natalizumab. Nature Reviews Drug Discovery, 4:510-519, 2005
Steinman L and Zamvil S. Transcriptional analysis of targets in multiple sclerosis. Nature Reviews in Immunology, 3:483-493, 2003
Stromnes I M, Goverman J M. Osteopontin-induced survival of T cells. Nat. Immunol. 2007 January;8(1):19-20.
Wang K X, Shi Y, Denhardt D T. (2007) Osteopontin regulates hindlimb-unloading-induced lymphoid organ atrophy and weight loss by modulating corticosteroid production. Proc Natl Acad Sci U S A. 2007 104:14777-82
Wang, K. X. 2008. Osteopontin: Role in Immune Regulation and Stress Responses. Doctoral Dissertation. Rutgers, The State University of New Jersey.
Wang, K X and Denhardt, D T (2008) Osteopontin: Role in immune regulation and stress responses. Cytokine & Growth Factor Reviews (in press)
Wang, K X, Shi, Y F, Kazanecki, C C and Denhardt, D T (2009) Plasma Osteopontin Modulates Chronic Restraint Stress-Induced Thymus Atrophy by Regulating Stress Hormones: Inhibition by an Anti-OPN Monoclonal Antibody. J Experimental Medicine (submitted)
Vogt M H, Lopatinskaya L, Smits M, Polman C H, Nagelkerken L. Elevated osteopontin levels in active relapsing-remitting multiple sclerosis. Ann Neurol. June; 53(6):819-22, 2003.
Yednock T, Cannon C, Fritz L, Sanchez-Madrid F, Steinman L, and Karin N. Prevention of experimental autoimmune encephalomyelitis by antibodies against a4b1 integrin. Nature, 356:63-66, 1992
Zhu B, Suzuki K, Goldberg H A, Rittling S R, Denhardt D T, McCulloch C A, Sodek J. Osteopontin modulates CD44-dependent chemotaxis of peritoneal macrophages through G-protein-coupled receptors: evidence of a role for an intracellular form of osteopontin. J Cell Physiol. 2004 January;198(1):155-67.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

TABLE 5

| mAB | Hn | Fb | Ratio | mAB | Hn | Fb | Ratio | mAB | Hn | Fb | | Comments |
|---|---|---|---|---|---|---|---|---|---|---|---|---|
| 47Eb5 | 0.261 | ? | | 50H9 | 1.164 | 0.265 | 4.392453 | 50H9 | 1.164 | 0.265 | 4.392453 | |
| 4AG9 | 2.039 | 3.000 | 0.6797 | 46H8 | 0.093 | 0.259 | 0.359073 | 23D7 | 0.296 | 0.186 | 1.591398 | |
| 5A4 | 0.362 | 3.000 | 0.1207 | 67B8 | 0.119 | 0.245 | 0.485714 | 10H4 | 3.000 | 0.180 | 16.66667 | |
| 7E3 | 0.857 | 3.000 | 0.2857 | 71A10 | 0.000 | 0.217 | 0 | 45C2 | 0.395 | 0.156 | 2.532051 | 45C2 Had low native |
| 39F10 | 3.000 | 3.000 | 1 | 28F3 | 0.103 | 0.194 | 0.530928 | 49C12 | 0.203 | 0.155 | 1.309677 | on my ELISA |
| 8A7 | 2.734 | 2.334 | 1.1714 | 66C8 | 0.091 | 0.191 | 0.47644 | 73C3 | 0.197 | 0.148 | 1.331081 | |
| 7B4 | 1.342 | 2.082 | 0.6446 | 23D7 | 0.296 | 0.186 | 1.591398 | 23F4 | 0.242 | 0.143 | 1.692308 | |
| 3D8 | 1.333 | 1.731 | 0.7701 | 10H4 | 3.000 | 0.180 | 16.66667 | 47B7 | 0.395 | 0.142 | 2.78169 | |
| 2A1 | 1.425 | 1.642 | 0.8678 | 45C2 | 0.395 | 0.156 | 2.532051 | 23F1 | 0.231 | 0.054 | 4.277778 | |
| 5C3 | 0.807 | 1.598 | 0.505 | 49C12 | 0.203 | 0.155 | 1.309677 | 21F12 | 0.488 | 0.043 | 11.34884 | |
| 1F8 | 0.408 | 1.302 | 0.3134 | 73C3 | 0.197 | 0.148 | 1.331081 | 1H3F7 | 3.000 | 0.040 | 75 | |
| 25C12 | 0.147 | 1.242 | 0.1184 | 23F4 | 0.242 | 0.143 | 1.692308 | 23F7 | 0.289 | 0.019 | 15.21053 | |
| 6A4 | 0.686 | 1.186 | 0.5784 | 47B7 | 0.395 | 0.142 | 2.78169 | 62D12 | 0.200 | 0.000 | | |
| 2D6 | ? | 1.140 | | 66H4 | 0.000 | 0.130 | 0 | 63D12 | 0.170 | 0.000 | | |
| 50F7 | 0.164 | 0.952 | 0.1723 | 15A3 | 0.000 | 0.129 | 0 | | | | | |
| 42B12 | 1.747 | 0.889 | 1.9651 | 73E4 | 0.114 | 0.128 | 0.890625 | | | | | |
| 10F6 | 0.498 | 0.869 | 0.5731 | 43A3 | 0.000 | 0.116 | 0 | | | | | |
| 67E9 | 0.039 | 0.688 | 0.0567 | 64G6 | 0.123 | 0.111 | 1.108108 | | | | | |
| 66E6 | 0.136 | 0.561 | 0.2424 | 68A3 | 0.000 | 0.106 | 0 | | | | | |
| 61B5 | 1.330 | 0.516 | 2.5775 | 5C11 | 0.000 | 0.102 | 0 | | | | | |
| 45E8 | 0.188 | 0.461 | 0.4078 | 2D4 | 0.000 | 0.054 | 0 | | | | | |
| 2G9 | 0.169 | 0.449 | 0.3764 | 23F1 | 0.231 | 0.054 | 4.277778 | | | | | |
| 64D5 | 0.127 | 0.436 | 0.2913 | 21F12 | 0.488 | 0.043 | 11.34884 | | | | | |
| 67C10 | 0.239 | 0.424 | 0.5637 | 4B5 | 0.106 | 0.041 | 2.585366 | | | | | |
| 23E11 | 0.873 | 0.400 | 2.1825 | 1H3F7 | 3.000 | 0.040 | 75 | | | | | |
| 66H7 | 0.041 | 0.379 | 0.1082 | 2A8 | 0.051 | 0.033 | 1.545455 | | | | | |
| 66G11 | 0.228 | 0.378 | 0.6032 | 23F7 | 0.289 | 0.019 | 15.21053 | | | | | |
| 66C3 | 0.150 | 0.368 | 0.4076 | 22H7 | 0.000 | 0.013 | 0 | | | | | |
| 66E10 | 0.088 | 0.365 | 0.2411 | 23E7 | 0.000 | 0.000 | | | | | | |
| 49B12 | 0.459 | 0.356 | 1.2893 | 52F2 | 0.126 | 0.000 | | | | | | |
| 64G7 | 0.321 | 0.324 | 0.9907 | 62D12 | 0.200 | 0.000 | | | | | | |
| 65C2 | 0.387 | 0.312 | 1.2404 | 63D12 | 0.170 | 0.000 | | | | | | |
| 66F4 | 0.195 | 0.306 | 0.6373 | 67F7 | 0.125 | 0.000 | | | | | | |
| 66A7 | 0.140 | 0.300 | 0.4667 | 67H9 | 0.208 | | | | | | | |
| 64E12 | 0.100 | 0.268 | 0.3731 | 69C7 | 0.037 | | | | | | | |
| | | | | 73A3 | 0.006 | | | | | | | |

What is claimed is:

1. A method for producing osteopontin-specific antibodies, comprising:
a) immunizing an osteopontin knock out mouse with an immunogenic amount of osteopontin or fragments thereof;
b) harvesting serum from said mouse; and
c) screening said serum for antibodies immunoreactive to osteopontin.

2. An antibody preparation produced by the method of claim 1.

3. A method for producing osteopontin-specific antibodies, comprising:
a) immunizing an osteopontin knock out mouse with an immunogenic amount of osteopontin or fragments thereof;
b) harvesting the spleen of said mouse and fusing said spleen cells with a myeloma cell line containing a mutation to facilitate isolation of fused spleen/myeloma cells;
c) culturing said fused cells in media containing a selection agent; and
d) screening said media for the presence of antibodies immunoreactive to osteopontin.

4. A monoclonal antibody produced by the method of claim 14 and shown in FIG. 29.

5. An osteopontin specific monoclonal antibody which recognizes at least one epitope shown in FIG. 29.

6. An anti-human osteopontin antibody selected from the group consisting of 42B12, 23F4, 45B5, 2D4, 2D6, 5A4, 7E3,23F1, 23F4, 10H4, 5A4, 7B4, 42B12 and 10F6.

7. A method for modulating autoimmune disease in a patient in need thereof, comprising administration of an effective amount of a monoclonal antibody immunologically specific for osteopontin.

8. The method of claim 7, wherein said antibody recognizes the amino terminus of osteopontin.

9. The method of claim 7, wherein said autoimmune disease is selected from the group consisting of multiple sclerosis, adjuvant-induced arthritis, eosinophila myalgia syndrome and systemic lupus erythematosous.

10. The method of claim 7, wherein said autoimmune disease is multiple sclerosis.

11. A method for modulating corticosteroid levels in a patient in need thereof, comprising administration of an effective amount of the monoclonal antibody of claim 5.
